SEQUENCE LISTING (36 sequences)

SEQ ID NO: 1 - protein, 266 aa, Pseudomonas
  receptor binding domain of Pseudomonas exotoxin A
MHLIPHWIPLVASLGLLAGGSSASAAEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQTQPRREKRWSEWASGKVLCLLDPLDGVYNYLAQQRCNLDDTWEGKIYRVLAGNPAKHDLDIKP

SEQ ID NO: 2 - protein, 153 aa, Pseudomonas
  Pseudomonas exotoxin A transcytosis domain
TVISHRLHFPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANADVVSLTCPVAAGECAGPADSGDALLERNY

SEQ ID NO: 3 - DNA, 1839 bp, Artificial Sequence
  Nucleotide sequence encoding Pseudomonas Exotoxin A
gccgaagaagctttcgacctctggaacgaatgcgccaaagcctgcgtgctcgacctcaaggacggcgtgcgttccagccgcatgagcgtcgacccggccatcgccgacaccaacggccagggcgtgctgcactactccatggtcctggagggcggcaacgacgcgctcaagctggccatcgacaacgccctcagcatcaccagcgacggcctgaccatccgcctcgaaggcggcgtcgagccgaacaagccggtgcgctacagctacacgcgccaggcgcgcggcagttggtcgctgaactggctggtaccgatcggccacgagaagccctcgaacatcaaggtgttcatccacgaactgaacgccggcaaccagctcagccacatgtcgccgatctacaccatcgagatgggcgacgagttgctggcgaagctggcgcgcgatgccaccttcttcgtcagggcgcacgagagcaacgagatgcagccgacgctcgccatcagccatgccggggtcagcgtggtcatggcccagacccagccgcgccgggaaaagcgctggagcgaatgggccagcggcaaggtgttgtgcctgctcgacccgctggacggggtctacaactacctcgcccagcaacgctgcaacctcgacgatacctgggaaggcaagatctaccgggtgctcgccggcaacccggcgaagcatgacctggacatcaaacccacggtcatcagtcatcgcctgcactttcccgagggcggcagcctggccgcgctgaccgcgcaccaggcttgccacctgccgctggagactttcacccgtcatcgccagccgcgcggctgggaacaactggagcagtgcggctatccggtgcagcggctggtcgccctctacctggcggcgcggctgtcgtggaaccaggtcgaccaggtgatccgcaacgccctggccagccccggcagcggcggcgacctgggcgaagcgatccgcgagcagccggagcaggcccgtctggccctgaccctggccgccgccgagagcgagcgcttcgtccggcagggcaccggcaacgacgaggccggcgcggccaacgccgacgtggtgagcctgacctgcccggtcgccgccggtgaatgcgcgggcccggcggacagcggcgacgccctgctggagcgcaactatcccactggcgcggagttcctcggcgacggcggcgacgtcagcttcagcacccgcggcacgcagaactggacggtggagcggctgctccaggcgcaccgccaactggaggagcgcggctatgtgttcgtcggctaccacggcaccttcctcgaagcggcgcaaagcatcgtcttcggcggggtgcgcgcgcgcagccaggacctcgacgcgatctggcgcggtttctatatcgccggcgatccggcgctggcctacggctacgcccaggaccaggaacccgacgcacgcggccggatccgcaacggtgccctgctgcgggtctatgtgccgcgctcgagcctgccgggcttctaccgcaccagcctgaccctggccgcgccggaggcggcgggcgaggtcgaacggctgatcggccatccgctgccgctgcgcctggacgccatcaccggccccgaggaggaaggcgggcgcctggagaccattctcggctggccgctggccgagcgcaccgtggtgattccctcggcgatccccaccgacccgcgcaacgtcggcggcgacctcgacccgtccagcatccccgacaaggaacaggcgatcagcgccctgccggactacgccagccagcccggcaaaccgccgcgcgaggacctgaag

SEQ ID NO: 4 - protein, 4 aa, Artificial Sequence
  Peptide recognized by Cathepsin GI
AAPF

SEQ ID NO: 5 - protein, 3 aa, Artificial Sequence
  Peptide recognized by Chymotrypsin I
GGF

SEQ ID NO: 6 - protein, 4 aa, Artificial Sequence
  Peptide recognized by Elastase I
AAPV

SEQ ID NO: 7 - protein, 3 aa, Artificial Sequence
  Peptide recognized by Subtilisin AI
GGL

SEQ ID NO: 8 - protein, 3 aa, Artificial Sequence
  Peptide recognized by SubtilisIn AII
AAL

SEQ ID NO: 9 - protein, 3 aa, Artificial Sequence
  Peptide recognized by Thrombin I
FVR

SEQ ID NO: 10 - protein, 3 aa, Artificial Sequence
  Peptide recognized by Urokinase I
VGR

SEQ ID NO: 11 - protein, 5 aa, Artificial Sequence
  VARIANT 5: Xaa = Any Amino Acid
  Peptide(s) recognized by Caspase-1
YVADX

SEQ ID NO: 12 - protein, 5 aa, Artificial Sequence
  VARIANT 2,3,5: Xaa = Any Amino Acid
  Peptide(s) recognized by Caspase-3
DXXDX

SEQ ID NO: 13 - protein, 9 aa, Artificial Sequence
  VARIANT 2: Xaa = Any Amino Acid
  VARIANT 3, 4, 5, 6, 7: Xaa = Any Amino Acid or absent
  VARIANT 9: Xaa = Any Amino Acid
  Peptide(s) recognized by Proprotein convertase 1
RXXXXXXRX

SEQ ID NO: 14 - protein, 9 aa, Artificial Sequence
  VARIANT 2: Xaa = Any Amino Acid
  VARIANT 3, 4, 5, 6, 7: Xaa = Any Amino Acid or absent
  VARIANT 9: Xaa = Any Amino Acid
  Peptide(s) recognized by Proprotein convertase 2
KXXXXXXRX

SEQ ID NO: 15 - protein, 6 aa, Artificial Sequence
  VARIANT 6: Xaa = Any Amino Acid
  Peptide(s) recognized by Proprotein convertase 4
GRTKRX

SEQ ID NO: 16 - protein, 5 aa, Artificial Sequence
  VARIANT 5: Xaa = Any Amino Acid
  Peptide(s) recognized by Proprotein convertase 4 (PACE 4)
RVRRX

SEQ ID NO: 17 - protein, 6 aa, Artificial Sequence
  VARIANT 6: Xaa = Any Amino Acid
  Peptide(s) recognized by Proprotein convertase 4 PACE 4
DRVRRX

SEQ ID NO: 18 - protein, 6 aa, Artificial Sequence
  VARIANT 2,6: Xaa = Any Amino Acid
  Peptide(s) recognized by Prolyloligopeptidase Endothelin cleaving enzyme with dipeptidyl-peptidase IV
PXWVPX

SEQ ID NO: 19 - protein, 4 aa, Artificial Sequence
  VARIANT 4: Xaa = Any Amino Acid
  Peptide(s) recognized by Signal Peptidase
WVAX

SEQ ID NO: 20 - protein, 4 aa, Artificial Sequence
  VARIANT 1,3,4: Xaa = Any Amino Acid
  Peptide(s) recognized by Neprilysin in combination with dipeptidyl-peptidase IV
XFXX

SEQ ID NO: 21 - protein, 4 aa, Artificial Sequence
  VARIANT 1,3,4: Xaa = Any Amino Acid
  Peptide(s) recognized by Neprilysin in combination with dipeptidyl-peptidase IV
XYXX

SEQ ID NO: 22 - protein, 4 aa, Artificial Sequence
  VARIANT 1,3,4: Xaa = Any Amino Acid
  Peptide(s) recognized by Neprilysin in combination with dipeptidyl-peptidase IV
XWXX

SEQ ID NO: 23 - protein, 12 aa, Artificial Sequence
  VARIANT 10: Xaa = Val, Ala or Pro
  VARIANT 12: Xaa = Ser, Pro or Ala
  Peptide(s) recognized by Renin in combination with dipeptidyl-peptidase IV
DRYIPFHLLXYX

SEQ ID NO: 24 - protein, 63 aa, homo sapiens
  Fc-binder domain of Protein G
VRQGTGNTTYKLVINGKTLKGETTTEAVDAATAEKVFKQYANDNGVDGEWTYDDATKTFTVTE

SEQ ID NO: 25 - protein, 68 aa, homo sapiens
  Protein A antibody-binding fragment
VRQGTGNTADNKFNKEQQNAFYEILHLPNLNWWQRNGFIQSLKDDPSQSANNLLAEAKKLNDAQAPKA

SEQ ID NO: 26 - protein, 365 aa, homo sapiens
  human FcRn
MGVPRPQPWALGLLLFLLPGSLGAESHLSLLYHLTAVSSPAPGTPAFWVSGWLGPQQYLSYNSLRGEAEPCGAWVWENQVSWYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVELESPAKSSVLVVGIVIGVLLLTAAAVGGALLWRRMRSGLPAPWISLRGDDTGVLLPTPGEAQDADLKDVNVIPATA

SEQ ID NO: 27 - protein, 224 aa, homo sapiens
  human Fc gamma RIII
HTCPPCPAPELLGGPSVFLFPPKPKDTLMISRTPEVTCVVVDVSHEDPQVKFNWYVDGVEVHNAKTKPREEQYNSTYRVVSVLTVLHQNWLDGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 233 aa, homo sapiens
  human Fc gamma RIII - beta
MWQLLLPTALLLLVSAGMRTEDLPKAVVFLEPQWYSVLEKDSVTLKCQGAYSPEDNSTQWFHNESLISSQASSYFIDAATVNDSGEYRCQTNLSTLSDPVQLEVHIGWLLLQAPRWVFKEEDPIHLRCHSWKNTALHKVTYLQNGKDRKYFHHNSDFHIPKATLKDSGSYFCRGLVGSKNVSSETVNITITQGLAVSTISSFSPPGYQVSFCLVMVLLFAVDTGLYFSVKTNI

SEQ ID NO: 29 - protein, 143 aa, homo sapiens
  antibody-binding domain of human Fc gamma RIII - beta
NLSTLSDPVQLEVHIGWLLLQAPRWVFKEEDPIHLRCHSWKNTALHKVTYLQNGKDRKYFHHNSDFHIPKATLKDSGSYFCRGLVGSKNIVSSETVNITITQGLAVSTISSFSPPGYQVSFCIVMVLLFAVDTYLYFSVKTNI

SEQ ID NO: 30 - protein, 7 aa, Artificial Sequence
  cleavable linker for ntPE-Fc gamma RIII carrier construct
RQPRGGL

SEQ ID NO: 31 - protein, 7 aa, Artificial Sequence
  cleavable linker for ntPE-Fc gamma RIII carrier construct
GGLRQPR

SEQ ID NO: 32 - protein, 7 aa, Artificial Sequence
  cleavable linker for ntPE-Fc gamma RIII carrier construct
RQPREGR

SEQ ID NO: 33 - protein, 7 aa, Artificial Sequence
  cleavable linker for ntPE-Fc gamma RIII carrier construct
RQPRVGR

SEQ ID NO: 34 - protein, 7 aa, Artificial Sequence
  cleavable linker for ntPE-Fc gamma RIII carrier construct
RQPRARR

SEQ ID NO: 35 - DNA, 1698 bp, Artificial Sequence
  Carrier Construct 1 (hGH)
atggccgaagaagctttcgacctctggaacgaatgcgccaaagcctgcgtgctcgacctcaaggacggcgtgcgttccagccgcatgagcgtcgacccggccatcgccgacaccaacggccagggcgtgctgcactactccatggtcctggagggcggcaacgacgcgctcaagctggccatcgacaacgccctcagcatcaccagcgacggcctgaccatccgcctcgaaggcggcgtcgagccgaacaagccggtgcgctacagctacacgcgccaggcgcgcggcagttggtcgctgaactggctggtaccgatcggccacgagaagccctcgaacatcaaggtgttcatccacgaactgaacgccggcaaccagctcagccacatgtcgccgatctacaccatcgagatgggcgacgagttgctggcgaagctggcgcgcgatgccaccttcttcgtcagggcgcacgagagcaacgagatgcagccgacgctcgccatcagccatgccggggtcagcgtggtcatggcccagacccagccgcgccgggaaaagcgctggagcgaatgggccagcggcaaggtgttgtgcctgctcgacccgctggacggggtctacaactacctcgcccagcaacgctgcaacctcgacgatacctgggaaggcaagatctaccgggtgctcgccggcaacccggcgaagcatgacctggacatcaaacccacggtcatcagtcatcgcctgcactttcccgagggcggcagcctggccgcgctgaccgcgcaccaggcttgccacctgccgctggagactttcacccgtcatcgccagccgcgcggctgggaacaactcgagcagtgcggctatccggtgcagcggctggtcgccctctacctggcggcgcggctgtcgtggaaccaggtcgaccaggtgatccgcaacgccctggccagccccggcagcggcggcgacctgggcgaagcgatccgcgagcagccggagcaggcccgtctggccctgaccctggccgccgccgagagcgagcgcttcgtccggcagggcaccggcaacgacgaggccggcgcggcaaacctgcagggaggattacgccagcctcgattcccgaccatcccgctgtcccgtctgttcgacaacgctatgctgcgtgctcaccgtctgcaccagctggctttcgacacctaccaggagttcgaagaagcatacatcccgaaagaacagaaatactccttcctgcaaaacccgcagacctccctgtgcttctccgaatcgatcccgaccccgtccaaccgtgaagaaacccagcagaaatccaacctggagctcctgcgtatctccctgctgctgatccagtcctggctcgagccggttcagttcctgcgttccgttttcgctaactccctggtttacggtgctagcgactccaacgtttacgacctgctgaaagacctggaagaaggtatccagaccctgatgggtcgtctggaagacggttccccgcgtaccggtcagatcttcaaacagacctactccaaattcgacaccaactcccacaacgacgacgctctgctgaaaaactacggtctgctgtactgcttccgtaaagacatggacaaagttgaaaccttcctgcgtatcgttcagtgccgttccgttgaaggttcctgcggtttctaa

SEQ ID NO: 36 - protein, 565 aa, Artificial Sequence
  Carrier Construct 1 (hGH)
MAEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQTQPRREKRWSEWASGKVLCLLDPLDGVYNYLAQQRCNLDDTWEGKIYRVLAGNPAKHDLDIKPTVISHRLHFPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANLQGGLRQPRFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

(19) United States
(12) Patent Application Publication
(10) Pub. No.: US 2007/0148131 A1
(43) Pub. Date: Jun. 28, 2007

(54) Methods and compositions for needleless delivery of binding partners

(75) Inventor: Randall J. Mrsny, Los Altos, CA (US)

Correspondence Address: JONES DAY, 222 EAST 41ST ST, NEW YORK, NY, 10017, US

(73) Assignee: Trinity Biosystems, Inc., Menlo Park, CA (US)

(21) Appl. No.: 11/635,241

(22) Filed: Dec. 5, 2006

Related U.S. Application Data
(60) Provisional application No. 60/742,633, filed on Dec. 5, 2005.

(51) Int. Cl. A61K 38/19; A61K 38/20; A61K 48/00; A61K 38/21

(52) U.S. Cl. 424/85.1; 424/85.2; 424/85.5; 424/85.6; 424/85.7; 514/12; 514/44

(57) ABSTRACT

The present invention relates, in part, to methods and compositions for needleless delivery of macromolecules to a subject. In one aspect, the methods and compositions involve administering to the subject a delivery construct comprising a carrier construct non-covalently bound to a binding partner, wherein the carrier construct comprises a receptor-binding domain, a transcytosis domain, and a macromolecule to which the binding partner non-covalently binds, wherein the binding partner binds to the macromolecule with a Kₐ that is at least about 10⁴ M⁻¹.

This application is entitled to and claims benefit under 35 U.S.C. § 119(e) of U.S. Provisional Application No. 60/742,633, filed Dec. 5, 2005, which is hereby incorporated by reference in its entirety.

### 1. FIELD OF THE INVENTION

The present invention relates, in part, to methods and compositions for needleless delivery of macromolecules to a subject. In one aspect, the methods and compositions involve administering to the subject a delivery construct comprising a carrier construct non-covalently bound to a binding partner, wherein the carrier construct comprises a receptor-binding domain, a transcytosis domain, and a macromolecule to which the binding partner non-covalently binds, wherein the binding partner binds to the macromolecule with a Kₐ that is at least about 10⁴ M⁻¹.

### 2. BACKGROUND

Advances in biochemistry and molecular biology have resulted identification and characterization of many therapeutic macromolecules, including, for example, growth horrnone, erythropoietin, insulin, IGF, and the like. Administration of these molecules can result in drastic improvements in quality of life for subjects afflicted with a wide range of ailments. Many of these macromolecules exist in serum as protein complexes, including, for example, growth hormone and IGF.

However, administration of these therapeutic macromolecules as protein complexes remains problematic. Currently, therapeutic macromolecules are typically administered by injection. Such injections require penetration of the subject's skin and tissues and are associated with pain. Further, penetration of the skin breaches one effective nonspecific mechanism of protection against infection, and thus can lead to potentially serious infection.

Accordingly, there is an unmet need for new methods and compositions that can be used to administer macromolecules to subjects without breaching the skin of the subject. This and other needs are met by the methods and compositions of the present invention.

### 3. SUMMARY OF THE INVENTION

The present invention provides delivery constructs for the administration of a binding partner or a binding partner-macromolecule complex to a subject. In one aspect of the invention, such delivery constructs comprise a carrier construct non-covalently bound to a binding partner. The carrier constructs of the present invention comprise: (a) a receptor-binding domain, (b) a transcytosis domain, and (c) a macromolecule to which the binding partner non-covalently binds, wherein the binding partner binds to the macromolecule with a Kₐ that is at least about 10⁴ M⁻¹. In certain embodiments, the carrier constructs further comprise a cleavable linker, wherein the cleavage at the cleavable linker separates the macromolecule from the remainder of the carrier construct. In one embodiment, the cleavable linker is cleavable by an enzyme that exhibits greater activity at a basal-lateral membrane of a polarized epithelial cell than at an apical membrane of the polarized epithelial cell. In an alternative embodiment, the cleavable linker is cleavable by an enzyme that exhibits greater activity in the plasma of a subject than at an apical membrane of the polarized epithelial cell of the subject. In embodiments of the invention where a binding partner-macromolecule complex is to be delivered to a subject, it is preferable that the carrier construct comprise a cleavable linker that separates the binding partner-macromolecule complex from the remainder of the carrier construct.

In some embodiments, the carrier construct comprises a macromolecule consisting of multiple subunits. In certain embodiments, the subunits of the macromolecule are separated by a linker of sufficient length to enable the subunits of the macromolecule to fold so that the macromolecule binds (e.g., covalently and/or non-covalently) to its binding partner. In other embodiments, a subunit of the macromolecule is linked to the remainder of the carrier construct and the construct is incubated with one or more other subunits under conditions that permit the subunits to associate and form the macromolecule. In these embodiments, the carrier construct that is used in accordance with the invention comprises both or all of the subunits of the macromolecule. In specific embodiments, the conditions permit the subunits of a macromolecule to associate in the same or substantially the same manner that they do in nature. In accordance with these embodiments, the binding partner is not a subunit of the macromolecule. For example, in a specific embodiment, the delivery construct is an IL-12 receptor-IL-12 delivery construct. In accordance with this embodiment, the carrier construct may comprise: (i) a receptor-binding domain, (ii) a transcytosis domain, (iii) a beta 1 subunit of IL-12 receptor, and (iv) a beta 2 subunit of IL-12 receptor. Such a carrier construct may be formed by incubating the beta 1 subunit of IL-12 receptor linked to the remainder of the carrier construct with beta 2 subunit of the IL-12 receptor under conditions that permit non-covalent bonds to form between the beta I and beta 2 subunits of IL-12 receptor. The carrier construct comprising the non-covalently associated IL-12 receptor subunits is the carrier and the binding partner is, e.g., IL-12.

In certain embodiments, a carrier construct comprises two macromolecules, wherein the second macromolecule is separated from the remainder of the carrier construct by a cleavable linker and cleavage at the cleavable linker separates the second macromolecule from the remainder of said construct. In some embodiments, a carrier construct comprises two macromolecules and two cleavable linkers, wherein the first cleavable linker separates the first macromolecule from the remainder of the construct and the second cleavable linker separates the second macromolecule from the remainder of the construct. The first and second cleavable linkers are, in some embodiments, the same and in other embodiments, different. In a specific embodiment, the second macromolecule is separated from the first macromolecule by a cleavable linker. In certain embodiments, the first macromolecule is a first polypeptide and said second macromolecule is a second polypeptide. In certain embodiments, the first polypeptide and the second polypeptide associate to form a multimer. In certain embodiments, the multimer is a dimer, tetramer, or octamer. In further embodiments, the dimer is an antibody. In further embodimetns, the tetramer is an antibody.

In accordance with the one aspect of the invention, the macromolecule of a carrier construct non-covalently binds to a binding partner of interest. In some embodiments, the macromolecules of the carrier construct binds to two or more binding partners of interest. In certain embodiments, the ratio of macromolecule to binding partner is 2:1, 3:1, 4:1 or 5:1. In specific embodiments, the macromolecule of the carrier construct specifically binds to the binding partner(s) of interest.

In particular embodiments, the macromolecule of the carrier construct is chosen because delivery of a particular macromolecule-binding partner complex(es) to a subject is desired. For example, in certain embodiments, a delivery construct is used to deliver a macromolecule-binding protein complex to a subject, wherein the macromolecule is growth hormone (GH) binding protein and binding partner is growth hormone (GH). GH that is circulated in the blood of a subject is found associated with binding proteins such as GH binding protein. Thus, the delivery of a GH-GH binding protein complex mimics the GH found in circulating blood. Further, the GH-GH binding protein complex increases the half-life of GH in the subject.

In one aspect, the delivery of a macromolecule-binding partner complex increases the half-life of the binding partner. In specific embodiments, the half-life the binding partner is increased 25%, 30%, 35%, 40%, 45%, 50%, 55%, 60%, 65%, 70%, 75%, 80%, 85%, 90%, 95% or more when it is non-covalently bound to the macromolecule as assessed by an assay known in the art. In another aspect, the delivery of a macromolecule-binding partner complex has a prophylactic and/or therapeutic benefit. In certain embodiments, the macromolecule-binding partner complex has a better prophylactic and/or therapeutic benefit than the binding partner as assessed by clinical and/or pathological symptoms of a disorder.

In certain embodiments, the macromolecule is selected from the group consisting of a nucleic acid, a peptide, a polypeptide, a protein, a small organic molecule and a lipid. In further embodiments, the polypeptide is selected from the group consisting of polypeptide hormones, cytokines, chemokines, growth factors, antibodies and clotting factors. In certain embodiments, the macromolecule is IGF-I, IL-2 receptor alpha, IL-18 binding protein, Shc-like protein (Sck) or the SH2 of Sck. In specific embodiments, the macromolecule is obtained or derived from the same species as the subject receiving the delivery construct. In preferred embodiments, the macromolecule is a human or humanized macromolecule, e.g., a human growth hormone, or a human or humanized antibody.

The receptor-binding domain of a carrier construct binds (preferably, specifically) to a cell surface receptor that is present on the apical membrane of an epithelial cell. The receptor-binding domain binds to the cell surface with sufficient affinity to allow endocytosis of the delivery construct. In a specific embodiment, the receptor-binding domain of a carrier construct binds to the α2-macroglobulin receptor, epidermal growth factor receptor, transferrin receptor, chemokine receptor, CD25, CD11B, CD11C, CD80, CD86, TNFα receptor, TOLL receptor, M-CSF receptor, GM-CSF receptor, scavenger receptor, or VEGF receptor. In certain embodiments, the receptor-binding domain of a carrier construct comprises a receptor-binding domain from Pseudomonas exotoxin A; cholera toxin; botulinum toxin; diptheria toxin; shiga toxin; shiga-like toxin; monoclonal antibodies; polyclonal antibodies; single-chain antibodies; TGF α; EGF; IGF-I; IGF-II; IGF-III; IL-1; IL-2; IL-3; IL-6; MIP-1a; MIP-1b; MCAF; or IL-8. In a specific embodiment, the receptor-binding domain of a carrier construct comprises Domain Ia of Pseudomonas exotoxin A.

The transcytosis domain of a carrier construct effects the transcytosis of macromolecules that have bound to a cell surface receptor present on the apical membrane of an epithelial cell. In certain embodiments, the transcytosis domain of a carrier construct comprises a transcytosis domain from Pseudomonas exotoxin A, botulinum toxin, diptheria toxin, pertussis toxin, cholera toxin, heat-labile E. coli enterotoxin, shiga toxin, or shiga-like toxin. In a specific embodiment, the transcytosis domain of a carrier construct comprises the Pseudomonas exotoxin A transcytosis domain.

Binding partners are the molecules/compounds (including macromolecules) that one desires to deliver to a subject. In accordance with one aspect of the invention, the binding partner can be any molecule (including macromolecules) that non-covalently binds to another molecule (e.g., a second macromolecule) that is known to one of skill in the art. In certain embodiments, the binding partner is a peptide, a polypeptide, a protein, a nucleic acid, a carbohydrate, a lipid, a glycoprotein, synthetic organic compound, inorganic compound, or any combination thereof. In specific embodiments, the binding partner is obtained or derived from the same species as the subject receiving the delivery construct. In preferred embodiments, the binding partner is a human or humanized macromolecule.

In accordance with the invention, for purposes herein, a species that is a binding partner can be a macromolecule and vice versa. For example, in the case of IL-12 and IL-12R, the binding partner can be IL-12 or the IL-12 receptor, and the macromolecule of the carrier construct can be IL-12 receptor or IL-12, respectively.

In accordance with one aspect of the invention, in certain embodiments, the binding partner-macromolecule interaction has an on-rate sufficient for association and retention during uptake and transport across epithelial cells and an off-rate sufficient for release of the binding partner once the binding partner-macromolecule complex has reached the basolateral surface. In other embodiments, the binding partner-macromolecule interaction has a similar on-rate and/or off-rate as that found in nature.

In another aspect, the present invention provides delivery constructs for delivering multi-subunit macromolecules (i.e., a delivery construct in which the binding partner is one subunit of a macromolecule and the macromolecule portion of carrier construct is another subunit of the carrier construct) to a subject. In particular, the present invention provides delivery constructs comprising: (i) a macromolecule subunit as a binding partner; and (ii) a carrier construct comprising a receptor-binding domain, a transcytosis domain, and a second subunit of the macromolecule to which the binding partner binds. In certain embodiments, the second subunit of the macromolecule non-covalently binds to the binding partner. In other words, the first and second subunits of the macromolecule non-covalently bind to each other. In other embodiments, the second subunit of the macromolecule covalently binds to the binding partner. In other words, the first and second subunits of the macromolecule covalently bind to each other. For example, the two subunits are covalently linked by one, two or more disulfide bonds. In yet other embodiments, the second subunit of the macromolecule non-covalently and covalently binds to the binding partner. In other words, the first and second subunits of the macromolecule non-covalently and covalently bind to each other.

Accordingly, in a specific embodiment, the present invention provides delivery constructs comprising: (i) a macromolecule subunit as a binding partner; and (ii) a carrier construct comprising a receptor-binding domain, a transcytosis domain, and a second subunit of the macromolecule to which the binding partner covalently binds. In accordance with this embodiment, the carrier construct and the binding partner are incubated under conditions that permit the subunits to associate and form the macromolecule. In a specific embodiment, the conditions permit the subunits of the macromolecule to associate in the same manner that they do in nature. For example, the invention encompasses delivery constructs comprising: (i) the p35 subunit of IL-12, and (ii) a carrier comprising a receptor-binding domain, a transcytosis domain, and the p40 subunit of IL-12. Such delivery constructs may be formed by incubating the p35 subunit of IL-12 with the carrier construct under conditions (e.g., mildly oxidizing conditions) that permit a disulfide bond(s) to form between the p35 and p40 subunits of IL-12. In certain embodiments, the carrier construct further comprises a cleavable linker, wherein the cleavage at the cleavable linker separates the macromolecule from the remainder of the carrier construct.

In another specific embodiment, the present invention provides delivery constructs comprising: (i) a macromolecule subunit as a binding partner; and (ii) a carrier construct comprising a receptor-binding domain, a transcytosis domain, and a second subunit of the macromolecule to which the binding partner non-covalently binds. In accordance with this embodiment, the carrier construct and the binding partner are incubated under conditions that permit the subunits to associate and form the macromolecule. In a specific embodiment, the conditions permit the subunits of the macromolecule to associate in the same manner that they do in nature. For example, the invention encompasses delivery constructs comprising: (i) the beta 1 subunit of IL-12 receptor, and (ii) a carrier comprising a receptor-binding domain, a transcytosis domain, and the beta 2 subunit of IL-12 receptor. Such delivery constructs may be formed by incubating the beta 1 subunit of IL-12 receptor with the carrier construct under conditions that permit non-covalently bonds to form between the beta 1 and beta 2 subunits of IL-12 receptor. In certain embodiments, the carrier construct further comprises a cleavable linker, wherein the cleavage at the cleavable linker separates the macromolecule from the remainder of the carrier construct.

In another specific embodiment, the present invention provides delivery constructs comprising: (i) a macromolecule subunit as a binding partner; and (ii) a carrier construct comprising a receptor-binding domain, a transcytosis domain, and a second subunit of the macromolecule to which the binding partner covalently and non-covalently binds. In accordance with this embodiment, the carrier construct and the binding partner are incubated under conditions that permit the subunits to associate and form the macromolecule. In a specific embodiment, the conditions permit the subunits of the macromolecule to associate in the same manner that they do in nature. In certain embodiments, the carrier construct further comprises a cleavable linker, wherein the cleavage at the cleavable linker separates the macromolecule from the remainder of the carrier construct.

The delivery constructs of the invention may be produced, for example, by incubating a carrier construct and a binding partner together under conditions permissible for binding of the binding partner to the macromolecule of the carrier construct. In certain embodiments, the delivery constructs are produced by incubating the binding partner and the carrier construct together under conditions permissible for non-covalent binding of the binding partner to the macromolecule of the carrier construct. In specific embodiments, the binding partner and the carrier construct are incubated together under physiological conditions. Optionally, the delivery constructs formed by such incubation may be separated from unbound carrier construct and/or unbound binding partner using techniques known to one of skill in the art.

The delivery constructs of the invention may also be produced, for example, by co-expressing a carrier construct and a binding partner in cells engineered to comprise a first polynucleotide comprising a first nucleotide sequence encoding the carrier construct and a second polynucleotide comprising a second nucleotide sequence encoding the binding partner. The delivery constructs produced by the cells may be purified. Further, the delivery constructs of the invention may be produced, for example, by co-administering to a subject a first composition and a second composition, wherein the first composition comprises a carrier construct and the second composition comprises a binding partner.

In a preferred embodiment, the delivery constructs of the invention are not produced by happenstance in a subject; that is, such complexes are not normally present in a subject unless administered to the subject. In another preferred embodiment, the delivery constructs of the invention are suitable for administration to a subject, preferably, a human subject. In another preferred embodiment, the delivery constructs of the invention are purified.

The present invention provides compositions comprising a delivery construct of the invention. In a specific embodiment, the invention provides compositions comprising a delivery construct of the invention and a pharmaceutically acceptable diluent, excipient, vehicle, or carrier. In certain embodiments, the compositions of the invention are pharmaceutical compositions.

The present invention provides methods for delivering a binding partner or macromolecule-binding partner complex to a subject, the methods comprising contacting an apical surface of a polarized epithelial cell of the subject with a delivery construct of the invention. The present invention also provides methods for delivering a binding partner or macromolecule-binding partner complex to the bloodstream of a subject, the method comprising contacting a delivery construct of the invention to an apical surface of a polarized epithelial cell of the subject, such that the binding partner or the macromolecule-binding partner complex is delivered to the bloodstream of the subject.

Further, the present invention provides methods for preventing, treating, managing and ameliorating a disorder in a subject, the methods comprising administering to the subject a delivery construct of the invention.

### 4. BRIEF DESCRIPTION OF THE DRAWINGS

FIG. 1 presents the amino acid sequence of an exemplary Pseudomonas exotoxin A (PE).

FIG. 2 shows a nucleotide sequence that encodes Carrier Construct 1 (SEQ ID NO:35), an exemplary Carrier Construct comprising human growth hormone (hGH).

FIGS. 3A and B show the amino acid sequence of Carrier Construct 1 (SEQ ID NO:36), an exemplary carrier construct comprising hGH.

FIG. 4 shows, at different time points, the concentration of human IgG present in the serum of mice administered the delivery construct comprising the Fc-binding portion of Protein G and human IgG.

FIG. 5 presents a graphical representation of serum glucose concentrations following administration of insulin aggregates conjugated to ntPE or PBS to female BALB/c mice. Administration is either by oral gavage or subcutaneous injection, and two different exemplary conjugates were tested to assess the effect of the ratio of ntPE to insulin complex on delivery.

### 5. DETAILED DESCRIPTION OF THE INVENTION

5.1. Definitions

Unless defined otherwise, all technical and scientific terms used herein have the meaning commonly understood by a person skilled in the art to which this invention belongs. As used herein, the following terms have the meanings ascribed to them unless specified otherwise.

A “ligand” is a compound that specifically binds to a target molecule. Exemplary ligands include, but are not limited to, an antibody, a cytokine, a substrate, a signaling molecule, and the like.

A “receptor” is compound that specifically binds to a ligand.

“Immunoassay” refers to a method of detecting an analyte in a sample involving contacting the sample with an antibody that specifically binds to the analyte and detecting binding between the antibody and the analyte. A variety of immunoassay formats may be used to select antibodies specifically immunoreactive with a particular protein. For example, solid-phase ELISA immunoassays are routinely used to select monoclonal antibodies specifically immunoreactive with a protein. See Harlow and Lane (1988) Antibodies, A Laboratory Manual, Cold Spring Harbor Publications, New York, for a description of immunoassay formats and conditions that can be used to determine specific immunoreactivity. In one example, an antibody that binds a particular antigen with an affinity (Kₘ) of about 10 μM specifically binds the antigen.

“Linker” refers to a molecule that joins two other molecules, either covalently, or through ionic, van der Waals or hydrogen bonds, e.g., a nucleic acid molecule that hybridizes to one complementary sequence at the 5′ end and to another complementary sequence at the 3′ end, thus joining two non-complementary sequences. A “cleavable linker” refers to a linker that can be degraded or otherwise severed to separate the two components connected by the cleavable linker. Cleavable linkers are generally cleaved by enzymes, typically peptidases, proteases, nucleases, lipases, and the like. Cleavable linkers may also be cleaved by environmental cues, such as, for example, changes in temperature, pH, salt concentration, etc. when there is such a change in environment following transcytosis of the delivery construct across a polarized epithelial membrane.

“Pharmaceutical composition” refers to a composition suitable for pharmaceutical use in an animal. A pharmaceutical composition comprises a pharmacologically effective amount of an active agent and a pharmaceutically acceptable carrier. “Pharmacologically effective amount” refers to that amount of an agent effective to produce the intended pharmacological result. “Pharmaceutically acceptable carrier” refers to any of the standard pharmaceutical carriers, vehicles, buffers, and excipients, such as a phosphate buffered saline solution, 5% aqueous solution of dextrose, and emulsions, such as an oil/water or water/oil emulsion, and various types of wetting agents and/or adjuvants. Suitable pharmaceutical carriers and formulations are described in Remington's Pharmaceutical Sciences, 21st Ed. 2005, Mack Publishing Co., Easton. A “pharmaceutically acceptable salt” is a salt that can be formulated into a compound for pharmaceutical use including, e.g., metal salts (sodium, potassium, magnesium, calcium, etc.) and salts of ammonia or organic amines.

Preferred pharmaceutical carriers depend upon the intended mode of administration of the active agent. Typical modes of administration include enteral (e.g., oral, intranasal, rectal, or vaginal) or parenteral (e.g., subcutaneous, intramuscular, intravenous or intraperitoneal injection; or topical (e.g., transdermal, or transmucosal administration).

“Small organic molecule” refers to organic molecules of a size comparable to those organic molecules generally used in pharmaceuticals. The term excludes organic biopolymers (e.g., proteins, nucleic acids, etc.). Preferred small organic molecules range in size up to about 5000 Da, up to about 2000 Da, or up to about 1000 Da.

The terms “subject” and “patient” are used interchangeably to refer to a human or non-human animal, including a mammal or a primate, that is administered a delivery construct.

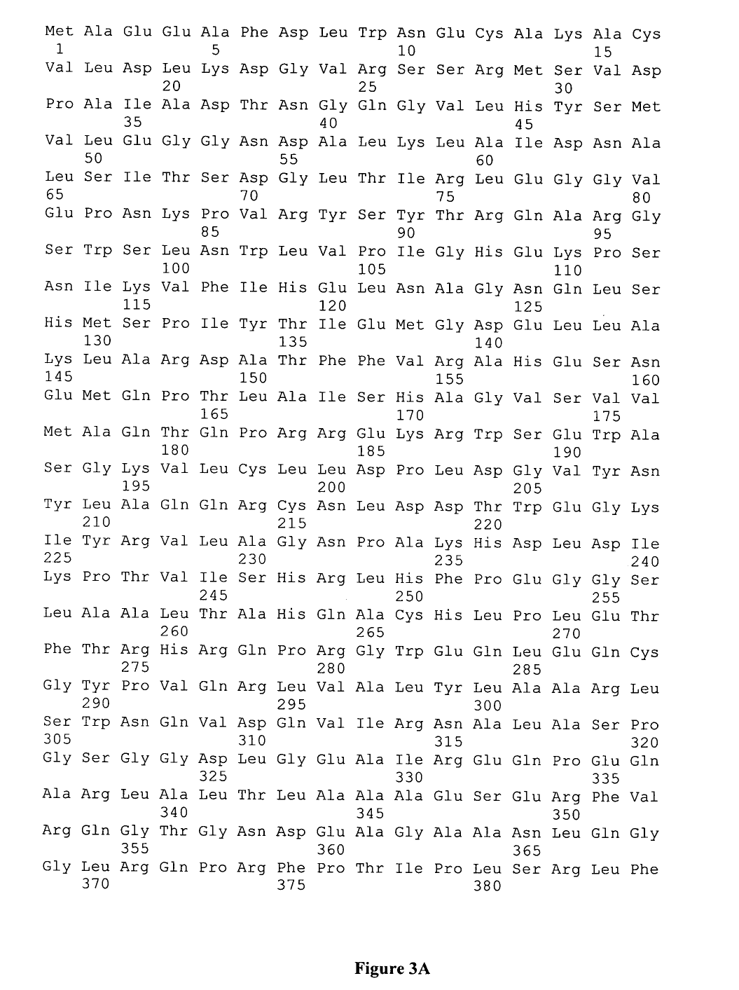

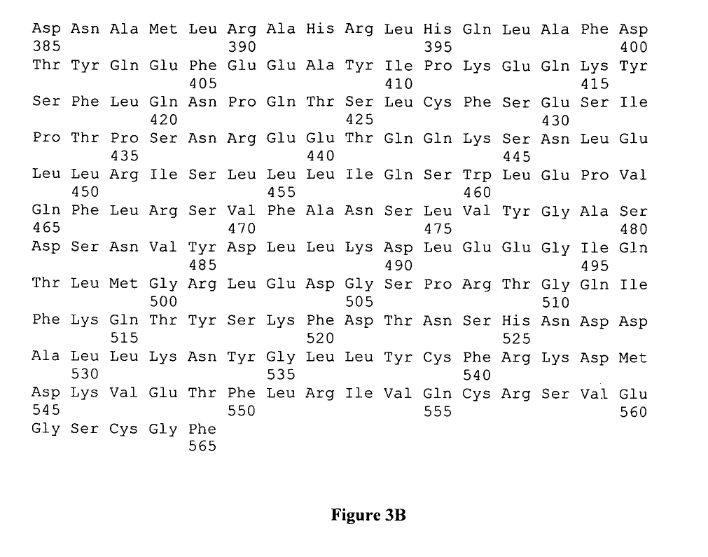

“Pseudomonas exotoxin A” or “PE” is secreted by Pseudomonas aeruginosa as a 67 kD protein composed of three prominent globular domains (Ia, II, and III) and one small subdomain (Ib) that connects domains II and III. See A. S. Allured et al., 1986, Proc. Natl. Acad. Sci. 83:1320-1324. Without intending to be bound to any particular theory or mechanism of action, domain Ia of PE is believed to mediate cell binding because domain Ia specifically binds to the low density lipoprotein receptor-related protein (“LRP”), also known as the α2-macroglobulin receptor (“α2-MR”) and CD-91. See M. Z. Kounnas et al., 1992, J. Biol. Chem. 267:12420-23. Domain Ia spans amino acids 1-252. Domain II of PE is believed to mediate transcytosis to the interior of a cell following binding of domain Ia to the α2-MR. Domain II spans amino acids 253-364. Certain portions of this domain may be required for secretion of PE from Pseudomonas aeruginosa after its synthesis. See, e.g., Vouloux et al., 2000, J. Bacterol. 182:4051-8. Domain Ib has no known function and spans amino acids 365-399. Domain III mediates cytotoxicity of PE and includes an endoplasmic reticulum retention sequence. PE cytotoxicity is believed to result from ADP ribosylation of elongation factor 2, which inactivates protein synthesis. Domain III spans amino acids 400-613 of PE. Deleting amino acid E553 (“ΔE553”) from domain III eliminates EF2 ADP ribosylation activity and detoxifies PE. PE having the mutation ΔE553 is referred to herein as “PEΔE553.” Genetically modified forms of PE are described in, e.g., U.S. Pat. Nos. 5,602,095; 5,512,658 and 5,458,878 Pseudomonas exotoxin, as used herein, also includes genetically modified, allelic, and chemically inactivated forms of PE within this definition. See, e.g., Vasil et al., 1986, Infect. Immunol. 52:538-48. Further, reference to the various domains of PE is made herein to the reference PE sequence presented as FIG. 3. However, one or more domain from modified PE, e.g., genetically or chemically modified PE, or a portion of such domains, can also be used in the chimeric immunogens of the invention so long as the domains retain functional activity. One of skill in the art can readily identify such domains of such modified PE is based on, for example, homology to the PE sequence exemplified in FIG. 3 and test for functional activity using, for example, the assays described below.

“Polynucleotide” refers to a polymer composed of nucleotide units. Polynucleotides include naturally occurring nucleic acids, such as deoxyribonucleic acid (“DNA”) and ribonucleic acid (“RNA”) as well as nucleic acid analogs. Nucleic acid analogs include those which include non-naturally occurring bases, nucleotides that engage in linkages with other nucleotides other than the naturally occurring phosphodiester bond or which include bases attached through linkages other than phosphodiester bonds. Thus, nucleotide analogs include, for example and without limitation, phosphorothioates, phosphorodithioates, phosphorotriesters, phosphoramidates, boranophosphates, methylphosphonates, chiral-methyl phosphonates, 2-O-methyl ribonucleotides, peptide-nucleic acids (PNAs), and the like. Such polynucleotides can be synthesized, for example, using an automated DNA synthesizer. The term “nucleic acid” typically refers to large polynucleotides. The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The direction of 5′ to 3′ addition of nucleotides to nascent RNA transcripts is referred to as the transcription direction. The DNA strand having the same sequence as an mRNA is referred to as the “coding strand”; sequences on the DNA strand having the same sequence as an mRNA transcribed from that DNA and which are located 5′ to the 5′-end of the RNA transcript are referred to as “upstream sequences”; sequences on the DNA strand having the same sequence as the RNA and which are 3′ to the 3′ end of the coding RNA transcript are referred to as “downstream sequences.”

“Complementary” refers to the topological compatibility or matching together of interacting surfaces of two polynucleotides. Thus, the two molecules can be described as complementary, and furthermore, the contact surface characteristics are complementary to each other. A first polynucleotide is complementary to a second polynucleotide if the nucleotide sequence of the first polynucleotide is substantially identical to the nucleotide sequence of the polynucleotide binding partner of the second polynucleotide, or if the first polynucleotide can hybridize to the second polynucleotide under stringent hybridization conditions. Thus, the polynucleotide whose sequence 5′-TATAC-3′ is complementary to a polynucleotide whose sequence is 5′-GTATA-3′.

The term “% sequence identity” is used interchangeably herein with the term “% identity” and refers to the level of amino acid sequence identity between two or more peptide sequences or the level of nucleotide sequence identity between two or more nucleotide sequences, when aligned using a sequence alignment program. For example, as used herein, 80% identity means the same thing as 80% sequence identity determined by a defined algorithm, and means that a given sequence is at least 80% identical to another length of another sequence. Exemplary levels of sequence identity include, but are not limited to, 60, 70, 80, 85, 90, 95, 98% or more sequence identity to a given sequence.

The term “% sequence homology” is used interchangeably herein with the term “% homology” and refers to the level of amino acid sequence homology between two or more peptide sequences or the level of nucleotide sequence homology between two or more nucleotide sequences, when aligned using a sequence alignment program. For example, as used herein, 80% homology means the same thing as 80% sequence homology determined by a defined algorithm, and accordingly a homologue of a given sequence has greater than 80% sequence homology over a length of the given sequence. Exemplary levels of sequence homology include, but are not limited to, 60, 70, 80, 85, 90, 95, 98% or more sequence homology to a given sequence.

Exemplary computer programs which can be used to determine identity between two sequences include, but are not limited to, the suite of BLAST programs, e.g., BLASTN, BLASTX, and TBLASTX, BLASTP and TBLASTN, publicly available on the Internet at the NCBI website. See also Altschul et al., 1990, J. Mol. Biol. 215:403-10 (with special reference to the published default setting, i.e., parameters w=4, t=17) and Altschul et al., 1997, Nucleic Acids Res., 25:3389-3402. Sequence searches are typically carried out using the BLASTP program when evaluating a given amino acid sequence relative to amino acid sequences in the GenBank Protein Sequences and other public databases. The BLASTX program is preferred for searching nucleic acid sequences that have been translated in all reading frames against amino acid sequences in the GenBank Protein Sequences and other public databases. Both BLASTP and BLASTX are run using default parameters of an open gap penalty of 11.0, and an extended gap penalty of 1.0, and utilize the BLOSUM-62 matrix. See id.

A preferred alignment of selected sequences in order to determine “% identity” between two or more sequences, is performed using for example, the CLUSTAL-W program in MacVector version 6.5, operated with default parameters, including an open gap penalty of 10.0, an extended gap penalty of 0.1, and a BLOSUM 30 similarity matrix.

“Polar Amino Acid” refers to a hydrophilic amino acid having a side chain that is uncharged at physiological pH, but which has at least one bond in which the pair of electrons shared in common by two atoms is held more closely by one of the atoms. Genetically encoded polar amino acids include Asn (N), Gln (Q) Ser (S) and Thr (T).

“Nonpolar Amino Acid” refers to a hydrophobic amino acid having a side chain that is uncharged at physiological pH and which has bonds in which the pair of electrons shared in common by two atoms is generally held equally by each of the two atoms (i.e., the side chain is not polar). Genetically encoded nonpolar amino acids include Ala (A), Gly (G), Ile (I), Leu (L), Met (M) and Val (V).

“Hydrophilic Amino Acid” refers to an amino acid exhibiting a hydrophobicity of less than zero according to the normalized consensus hydrophobicity scale of Eisenberg et al., 1984, J. Mol. Biol. 179:125-142. Genetically encoded hydrophilic amino acids include Arg (R), Asn (N), Asp (D), Glu (E), Gln (Q), His (H), Lys (K), Ser (S) and Thr (T).

“Hydrophobic Amino Acid” refers to an amino acid exhibiting a hydrophobicity of greater than zero according to the normalized consensus hydrophobicity scale of Eisenberg et al., 1984, J. Mol. Biol. 179:125-142. Genetically encoded hydrophobic amino acids include Ala (A), Gly (G), Ile (I), Leu (L), Met (M), Phe (F), Pro (P), Trp (W), Tyr (Y) and Val (V).

“Acidic Amino Acid” refers to a hydrophilic amino acid having a side chain pK value of less than 7. Acidic amino acids typically have negatively charged side chains at physiological pH due to loss of a hydrogen ion. Genetically encoded acidic amino acids include Asp (D) and Glu (E).

“Basic Amino Acid” refers to a hydrophilic amino acid having a side chain pK value of greater than 7. Basic amino acids typically have positively charged side chains at physiological pH due to association with a hydrogen ion. Genetically encoded basic amino acids include Arg (R), His (H) and Lys (K).

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Amplification” refers to any means by which a polynucleotide sequence is copied and thus expanded into a larger number of polynucleotide molecules, e.g., by reverse transcription, polymerase chain reaction, ligase chain reaction, and the like.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

“Probe,” when used in reference to a polynucleotide, refers to a polynucleotide that is capable of specifically hybridizing to a designated sequence of another polynucleotide. A probe specifically hybridizes to a target complementary polynucleotide, but need not reflect the exact complementary sequence of the template. In such a case, specific hybridization of the probe to the target depends on the stringency of the hybridization conditions. Probes can be labeled with, e.g., chromogenic, radioactive, or fluorescent moieties and used as detectable moieties. In instances where a probe provides a point of initiation for synthesis of a complementary polynucleotide, a probe can also be a primer.

“Hybridizing specifically to” or “specific hybridization” or “selectively hybridize to”, refers to the binding, duplexing, or hybridizing of a nucleic acid molecule preferentially to a particular nucleotide sequence under stringent conditions when that sequence is present in a complex mixture (e.g., total cellular) DNA or RNA.

The term “stringent conditions” refers to conditions under which a probe will hybridize preferentially to its target subsequence, and to a lesser extent to, or not at all to, other sequences. “Stringent hybridization” and “stringent hybridization wash conditions” in the context of nucleic acid hybridization experiments such as Southern and northern hybridizations are sequence dependent, and are different under different environmental parameters. An extensive guide to the hybridization of nucleic acids can be found in Tijssen, 1993, Laboratory Techniques in Biochemistry and Molecular Biology—Hybridization with Nucleic Acid Probes, part I, chapter 2, “Overview of principles of hybridization and the strategy of nucleic acid probe assays”, Elsevier, N.Y.; Sambrook et al., 2001, Molecular Cloning: A Laboratory Manual, Cold Spring Harbor Laboratory, 3^{rd} ed., NY; and Ausubel et al., eds., Current Edition, Current Protocols in Molecular Biology, Greene Publishing Associates and Wiley Interscience, NY.

Generally, highly stringent hybridization and wash conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Very stringent conditions are selected to be equal to the Tm for a particular probe.

One example of stringent hybridization conditions for hybridization of complementary nucleic acids which have more than about 100 complementary residues on a filter in a Southern or northern blot is 50% formalin with 1 mg of heparin at 42° C., with the hybridization being carried out overnight. An example of highly stringent wash conditions is 0.15 M NaCl at 72° C. for about 15 minutes. An example of stringent wash conditions is a 0.2X SSC wash at 65° C. for 15 minutes. See Sambrook et al. for a description of SSC buffer. A high stringency wash can be preceded by a low stringency wash to remove background probe signal. An exemplary medium stringency wash for a duplex of, e.g., more than about 100 nucleotides, is 1×SSC at 45° C. for 15 minutes. An exemplary low stringency wash for a duplex of, e.g., more than about 100 nucleotides, is 4-6×SSC at 40° C. for 15 minutes. In general, a signal to noise ratio of 2×(or higher) than that observed for an unrelated probe in the particular hybridization assay indicates detection of a specific hybridization.

“Peptide” refers to a compound composed of two or more amino acid residues linked via peptide bonds.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer. Conventional notation is used herein to portray polypeptide sequences; the beginning of a polypeptide sequence is the amino-terminus, while the end of a polypeptide sequence is the carboxyl-terminus.

The term “protein” typically refers to large polypeptides, for example, polypeptides comprising more than about 50 amino acids. The term “protein” can also refer to dimers, trimers, and multimers that comprise more than one polypeptide.

In the context of the interaction between to macromolecules (e.g., a binding partner and a macromolecule of a carrier construct), the term “specifically binds” and analogous terms refer to the binding of a macromolecule to another macromolecule with higher affinity than to any cross-reactive antigen as determined using experimental techniques, such as immunoassays (e.g., radioimmunoassays (RIA) and enzyme-linked immunosorbent assays (ELISAs)) and BIAcore. See, e.g., Paul, ed., 1989, Fundamental Immunology Second Edition, Raven Press, New York at pages 332-336 for a discussion regarding antibody specificity. For example, antibody binds specifically to a particular antigen when under designated conditions, the antibody binds preferentially to the particular antigen and does not bind in a significant amount to other antigens present in a sample.

“Conservative substitution” refers to the substitution in a polypeptide of an amino acid with a functionally similar amino acid. The following six groups each contain amino acids that are conservative substitutions for one another:
Alanine (A), Serine (S), and Threonine (T)
Aspartic acid (D) and Glutamic acid (E)
Asparagine (N) and Glutamine (Q)
Arginine (R) and Lysine (K)
Isoleucine (I), Leucine (L), Methionine (M), and Valine (V)
Phenylalanine (F), Tyrosine (Y), and Tryptophan (W).

The term “about,” as used herein, unless otherwise indicated, refers to a value that is no more than 10% above or below the value being modified by the term. For example, the term “about 5 μg/kg” means a range of from 4.5 μg/kg to 5.5 μg/kg. As another example, “about 1 hour” means a range of from 48 minutes to 72 minutes.

A “disorder” refers to a condition, preferably a pathological condition, in a subject.

A “purified” molecule (e.g., a delivery construct or carrier construct) is substantially free of cellular material or other contaminating proteins (e.g., unbound carrier construct and unbound binding partner in the context of a delivery construct) from the cell or tissue source from which the molecule (e.g., a delivery construct or carrier construct) is derived. The language “substantially free of cellular material” includes preparations of a molecule (e.g., a delivery construct or carrier construct) in which the molecule (e.g., a delivery construct or carrier construct) is separated from cellular components of the cells from which it is recombinantly produced. Thus, a molecule (e.g., a delivery construct or carrier construct) that is substantially free of cellular material includes preparations of the molecule having less than about 30%, 20%, 10%, or 5% (by dry weight) of heterologous protein (also referred to herein as a “contaminating protein”) and/or unbound carrier construct and unbound binding partner. When the molecule (e.g., a delivery construct or carrier construct) is recombinantly produced, it is also preferably substantially free of culture medium, i.e., culture medium represents less than about 20%, 10%, or 5% of the volume of the molecule (e.g., a delivery construct or carrier construct) preparation. In a specific embodiment, a delivery construct of the invention is purified. In another specific embodiment, a carrier construct of the invention is purified. In another specific embodiment, a binding partner of the invention is purified.

An “isolated” polynucleotide is one which is separated from other nucleic acid molecules which are present in the natural source of the polynucleotide. Moreover, an “isolated” polynucleotide, such as a cDNA molecule, can be substantially free of other cellular material, or culture medium when produced by recombinant techniques, or substantially free of chemical precursors or other chemicals when chemically synthesized. In certain embodiments, an “isolated” polynucleotide is a nucleic acid molecule that is recombinantly expressed in a heterologous cell.

The terms “manage,” “managing,” and “management” refer to the beneficial effects that a subject derives from a therapy (e.g., a prophylactic or therapeutic agent), which does not result in a cure of the disorder. In certain embodiments, a subject is administered one or more therapies (e.g., prophylactic or therapeutic agents, such as an antibody of the invention) to “manage” a disorder one or more symptoms thereof so as to prevent the progression or worsening of the disorder.

The terms “prevent,” “preventing,” and “prevention” in the context of administering a therapy to a subject refer to the total or partial inhibition of the disorder, or the total or partial inhibition of the development, onset or progression of the disorder and/or a symptom thereof in a subject.

The term “therapy” refers to any protocol, method and/or agent that can be used in the prevention, management, treatment and/or amelioration of a disorder or a symptom thereof. In certain embodiments, the terms “therapies” and “therapy” refer to a biological therapy, supportive therapy, and/or other therapies useful in the prevention, management, treatment and/or amelioration of a disorder or a symptom thereof known to one of skill in the art such as medical personnel. In a specific embodiment, a delivery construct is a therapy.

The terms “treat,” “treatment” and “treating” in the context of administration of a therapy to a subject refer to the reduction or amelioration of the progression, severity, and/or duration of a disorder or a symptom thereof.

The term “analog” in the context of a proteinaceous agent (e.g., a peptide, polypeptide, protein or antibody) refers to a proteinaceous agent that possesses a similar or identical function as a second proteinaceous agent but does not necessarily comprise a similar or identical amino acid sequence or structure of the second proteinaceous agent. A proteinaceous agent that has a similar amino acid sequence refers to a proteinaceous agent that satisfies at least one of the following: (a) a proteinaceous agent having an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the amino acid sequence of a second proteinaceous agent; (b) a proteinaceous agent encoded by a nucleotide sequence that hybridizes under stringent conditions to a nucleotide sequence encoding a second proteinaceous agent of at least 20 amino acid residues, at least 30 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, or at least 150 amino acid residues; and (c) a proteinaceous agent encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the nucleotide sequence encoding a second proteinaceous agent. A proteinaceous agent with similar structure to a second proteinaceous agent refers to a proteinaceous agent that has a similar secondary, tertiary or quaternary structure of the second proteinaceous agent. The structure of a proteinaceous agent can be determined by methods known to those skilled in the art, including but not limited to, X-ray crystallography, nuclear magnetic resonance, and crystallographic electron microscopy. The term “derivative” in the context of a proteinaceous agent (e.g., proteins, polypeptides, peptides, and antibodies) refers to a proteinaceous agent that comprises the amino acid sequence which has been altered by the introduction of amino acid residue substitutions, deletions, and/or additions. The term “derivative” as used herein also refers to a proteinaceous agent which has been modified, i.e., by the covalent attachment of a type of molecule to the proteinaceous agent. For example, but not by way of limitation, a derivative of a proteinaceous agent may be produced, e.g., by glycosylation, acetylation, pegylation, phosphorylation, amidation, derivatization by known protecting/blocking groups, proteolytic cleavage, linkage to a cellular ligand or other protein, etc. A derivative of a proteinaceous agent may also be produced by chemical modifications using techniques known to those of skill in the art, including, but not limited to specific chemical cleavage, acetylation, formylation, metabolic synthesis of tunicamycin, etc. Further, a derivative of a proteinaceous agent may contain one or more non-classical amino acids. A derivative of a proteinaceous agent possesses an identical function(s) as the proteinaceous agent from which it was derived.

The term “fragment” in the context of a proteinaceous agent refers to a peptide or polypeptide comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least contiguous 80 amino acid residues, at least contiguous 90 amino acid residues, at least contiguous 100 amino acid residues, at least contiguous 125 amino acid residues, at least 150 contiguous amino acid residues, at least contiguous 175 amino acid residues, at least contiguous 200 amino acid residues, or at least contiguous 250 amino acid residues of the amino acid sequence of a second peptide, polypeptide, or protein. In a specific embodiment, a fragment retains one or more functions of the peptide, polypeptide or protein from which it is derived.

The term “transcytosis” and analogous terms refer to the transport of macromolecular cargo from one side of a cell (e.g., the apical side of an epithelial cell) to the other side of the cell (e.g., the basolateral side of an epithelial cell) within a membrane membrane-bounded carrier(s). See, e.g., Tuma et al., 2003, Physiol. Rev. 83:871-932, which is incorporated herein in its entirety, for a review on transcytosis.

The term “endocytosis” and analogous terms refer to the process by which cells internalize macromolecules and fluid.

5.2. Delivery Constructs

In one embodiment, the delivery constructs of the present invention comprise a binding partner non-covalently bound to a carrier construct that comprises a receptor-binding domain, a transcytosis domain and a macromolecule to which the binding partner non-covalently binds, wherein the binding partner binds to the macromolecule with a Kₐ that is at least about 10⁴ M⁻¹. The non-covalent bond between the binding partner and macromolecule of the carrier construct may be the result of a single non-covalent bond or, preferably, multiple non-covalent bonds. Non-limiting examples of non-covalent bonds include hydrogen bonds, ionic bonds, van der Waals interactions, and hydrophobic bonds. In another embodiment, the present invention provides delivery constructs comprising: (i) a macromolecule subunit as a binding partner; and (ii) a carrier construct comprising a receptor-binding domain, a transcytosis domain, and a second subunit of the macromolecule to which the binding partner covalently binds. In accordance with this embodiment, the carrier construct and the binding partner are incubated under conditions that permit the subunits to associate and form the macromolecule. In a specific embodiment, the conditions permit the subunits of the macromolecule to associate in the same manner that they do in nature.

In another embodiment, the present invention provides delivery constructs comprising: (i) a macromolecule subunit as a binding partner; and (ii) a carrier construct comprising a receptor-binding domain, a transcytosis domain, and a second subunit of the macromolecule to which the binding partner non-covalently binds. In accordance with this embodiment, the carrier construct and the binding partner are incubated under conditions that permit the subunits to associate and form the macromolecule. In a specific embodiment, the conditions permit the subunits of the macromolecule to associate in the same manner that they do in nature.

In another embodiment, the present invention provides delivery constructs comprising: (i) a macromolecule subunit as a binding partner; and (ii) a carrier construct comprising a receptor-binding domain, a transcytosis domain, and a second subunit of the macromolecule to which the binding partner covalently and non-covalently binds. In accordance with this embodiment, the carrier construct and the binding partner are incubated under conditions that permit the subunits to associate and form the macromolecule. In a specific embodiment, the conditions permit the subunits of the macromolecule to associate in the same manner that they do in nature.

The delivery constructs of the invention offer several advantages over conventional techniques for local or systemic delivery of a binding partner, a binding partner-macromolecule complex and/or a macromolecule to a subject. Foremost among such advantages is the ability to deliver the binding partner, a binding partner-macromolecule complex and/or a macromolecule without using a needle to puncture the skin of the subject. Many subjects require repeated, regular doses of a binding partner. For example, individuals with growth hormone (GH) deficiency must inject this protein hormone several times per week to stimulate the desired growth outcome. Such subjects' quality of life would be greatly improved if the delivery of GH or GH-GH binding protein complex could be accomplished without injection, by avoiding pain or potential complications associated therewith.

5.3. Carrier Constructs

In one embodiment, the carrier constructs of the invention comprise the following structural elements, each element imparting particular functions to the carrier construct: (1) a “receptor-binding domain” that functions as a ligand for a cell surface receptor and that mediates binding of the construct to a cell; (2) a “transcytosis domain” that mediates transcytosis from a lumen bordering the apical surface of a mucous membrane to the basal-lateral side of a mucous membrane; and (3) the macromolecule to which a binding partner non-covalently binds with a Kₐ that is at least about 10⁴ M⁻¹. In certain embodiments, the carrier construct comprises these structural elements in the order listed above from N-terminus to C-terminus. Optionally, the carrier construct further comprises a cleavable linker that connects the macromolecule to the remainder of the carrier construct.

In another embodiment, the carrier constructs of the invention comprise the following structural elements, each element imparting particular functions to the carrier construct: (i) a “receptor-binding domain” that functions as a ligand for a cell surface receptor and that mediates binding of the construct to a cell, (ii) a transcytosis domain that mediates transcytosis from a lumen bordering the apical surface of a mucous membrane to the basal-lateral side of a mucous membrane, and (iii) a subunit of the macromolecule to which the binding partner non-covalently binds. In certain embodiments, the carrier construct comprises these structural elements in the order listed above from N-terminus to C-terminus. Optionally, the carrier construct further comprises a cleavable linker that connects the macromolecule to the remainder of the carrier construct.

In another embodiment, the carrier constructs of the invention comprise the following structural elements, each element imparting particular functions to the carrier construct: (i) a “receptor-binding domain” that functions as a ligand for a cell surface receptor and that mediates binding of the construct to a cell, (ii) a transcytosis domain that mediates transcytosis from a lumen bordering the apical surface of a mucous membrane to the basal-lateral side of a mucous membrane, and (iii) a subunit of the macromolecule to which the binding partner covalently binds with a Kₐ that is at least about 10⁴ M^{31 1}. In certain embodiments, the carrier construct comprises these structural elements in the order listed above from N-terminus to C-terminus. Optionally, the carrier construct further comprises a cleavable linker that connects the macromolecule to the remainder of the carrier construct.

In another embodiment, the carrier constructs of the invention comprise the following structural elements, each element imparting particular functions to the carrier construct: (i) a “receptor-binding domain” that functions as a ligand for a cell surface receptor and that mediates binding of the construct to a cell, (ii) a transcytosis domain that mediates transcytosis from a lumen bordering the apical surface of a mucous membrane to the basal-lateral side of a mucous membrane, and (iii) a subunit of the macromolecule to which the binding partner non-covalently and covalently binds. In certain embodiments, the carrier construct comprises these structural elements in the order listed above from N-terminus to C-terminus. Optionally, the carrier construct further comprises a cleavable linker that connects the macromolecule to the remainder of the carrier construct.

Generally, the carrier constructs of the present invention are polypeptides that have structural domains corresponding to domains Ia and II of PE. These structural domains perform certain functions, including, but not limited to, cell recognition and transcytosis, that correspond to the functions of the domains of PE.

In addition to the portions of the molecule that correspond to PE functional domains, the carrier constructs of this invention can further comprise a macromolecule for delivery to a biological compartment of a subject. The macromolecule can be introduced into any portion of the carrier construct that does not disrupt a cell-binding or transcytosis activity. In certain embodiments, the macromolecule is connected with the remainder of the carrier construct with a cleavable linker. In embodiments where a macromolecule-binding partner complex is to be delivered to a subject, it is preferable that the carrier construct comprises a cleavable linker that separates the binding partner-macromolecule complex from the remainder of the carrier construct.

Furthermore, many embodiments of the carrier constructs can be constructed and expressed in recombinant systems. Recombinant technology allows one to make a carrier construct having an insertion site designed for introduction of any suitable macromolecule. Such insertion sites allow the skilled artisan to quickly and easily produce carrier constructs for delivery of other binding partners and/or macromolecule-binding partner complexes, should the need to do so arise.

In addition, connection of the macromolecule to the remainder of the carrier construct with a linker that is cleaved by an enzyme present at a basal-lateral membrane of an epithelial cell allows the macromolecule to be liberated from the carrier construct and released from the remainder of the carrier construct soon after transcytosis across the epithelial membrane. Such liberation reduces the probability of induction of an immune response against the macromolecule. It also allows the macromolecule to interact with its target free from the remainder of the carrier construct.

Other advantages of the carrier constructs of the invention will be apparent to those of skill in the art.

In certain embodiments, the invention provides a carrier construct that comprises a receptor binding domain, a transcytosis domain, a macromolecule to which the binding partner covalently and/or non-covalently binds, and a cleavable linker. Cleavage at the cleavable linker separates the macromolecule from the remainder of the construct. The cleavable linker is cleavable by an enzyme that is present at a basal-lateral membrane of a polarized epithelial cell or in the plasma of a subject. In certain embodiments, the enzyme that is at a basal-lateral membrane of a polarized epithelial cell exhibits higher activity on the basal-lateral side of a polarized epithelial cell than it does on the apical side of the polarized epithelial cell. In certain embodiments, the enzyme that is in the plasma of the subject exhibits higher activity in the plasma than it does on the apical side of a polarized epithelial cell. In such embodiments, the activity of the cleaving enzyme can be greater because, for example, the cleaving enzyme is more active on, for example, the basal-lateral side of the polarized epithelial cell, or, for example, because the cleaving enzyme is expressed at a higher concentration on, for example, the basal-lateral side of the polarized epithelial cell, or both.

In certain embodiments, the carrier construct further comprises a second cleavable linker. In certain embodiments, the first and/or the second cleavable linker comprises an amino acid sequence that is selected from the group consisting of Ala-Ala-Pro-Phe (SEQ ID NO.:4), Gly-Gly-Phe (SEQ ID NO.:5), Ala-Ala-Pro-Val (SEQ ID NO.:6), Gly-Gly-Leu (SEQ ID NO.:7), Ala-Ala-Leu (SEQ ID NO.:8), Phe-Val-Arg (SEQ ID NO.:9), Val-Gly-Arg (SEQ ID NO.:10). In certain embodiments, the first and/or the second cleavable linker comprises an amino acid sequence that is selected from the group consisting of Ala-Ala-Pro-Phe (SEQ ID NO.:4), Gly-Gly-Phe (SEQ ID NO.:5), Ala-Ala-Pro-Val (SEQ ID NO.:6), Gly-Gly-Leu (SEQ ID NO.:7), Ala-Ala-Leu (SEQ ID NO.:8), Phe-Val-Arg (SEQ ID NO.:9), Val-Gly-Arg (SEQ ID NO.:10) and is cleavable by an enzyme that exhibits higher activity on the basal-lateral side of a polarized epithelial cell than it does on the apical side of the polarized epithelial cell. In certain embodiments, the first and/or the second cleavable linker comprises an amino acid sequence that is selected from the group consisting of Ala-Ala-Pro-Phe (SEQ ID NO.:4), Gly-Gly-Phe (SEQ ID NO.:5), Ala-Ala-Pro-Val (SEQ ID NO.:6), Gly-Gly-Leu (SEQ ID NO.:7), Ala-Ala-Leu (SEQ ID NO.:8), Phe-Val-Arg (SEQ ID NO.:9), Val-Gly-Arg (SEQ ID NO.:10) and is cleavable by an enzyme that exhibits higher activity in the plasma than it does on the apical side of a polarized epithelial cell.

In certain embodiments, the enzyme that is present at a basal-lateral membrane of a polarized epithelial cell is selected from the group consisting of Cathepsin GI, Chymotrypsin I, Elastase I, Subtilisin AI, Subtilisin AII, Thrombin I, and Urokinase I.

In certain embodiments, the receptor binding domain is selected from the group consisting of receptor binding domains from Pseudomonas exotoxin A, cholera toxin, botulinum toxin, diptheria toxin, shiga toxin, or shiga-like toxin; monoclonal antibodies; polyclonal antibodies; single-chain antibodies; TGF α; EGF; IGF-I; IGF-II; IGF-III; IL-1; IL-2; IL-3; IL-6; MIP-1a; MIP-1b; MCAF; and IL-8. In certain embodiments, the receptor binding domain binds to a cell-surface receptor that is selected from the group consisting of α2-macroglobulin receptor, epidermal growth factor receptor, transferrin receptor, chemokine receptor, CD25, CD11B, CD11C, CD80, CD86, TNFα receptor, TOLL receptor, M-CSF receptor, GM-CSF receptor, scavenger receptor, and VEGF receptor. In further embodiments, the receptor binding domain of Pseudomonas exotoxin A is Domain Ia of Pseudomonas exotoxin A. In yet further embodiments, the receptor binding domain of Pseudomonas exotoxin A has an amino acid sequence that is SEQ ID NO.:1.

In certain embodiments, the transcytosis domain is selected from the group consisting of transcytosis domains from Pseudomonas exotoxin A, botulinum toxin, diptheria toxin, pertussis toxin, cholera toxin, heat-labile E. coli enterotoxin, shiga toxin, and shiga-like toxin. In further embodiments, the transcytosis domain is Pseudomonas exotoxin A transcytosis domain. In still further embodiments, the Pseudomonas exotoxin A transcytosis domain has an amino acid sequence that is SEQ ID NO.:2.

In certain embodiments, the macromolecule of the carrier construct is chosen so that it non-covalently binds to the binding partner(s) of interest. In some embodiments, the macromolecule of the carrier construct binds to two or more binding partner(s) of interest. For example, in certain embodiments, the ratio of macromolecule to binding partner is 2:1, 3:1, 4:1 or 5:1. In specific embodiments, the macromolecule of the carrier construct specifically binds to the binding partner(s) of interest.

In particular embodiments, the macromolecule of the carrier construct is chosen because delivery of a particular macromolecule-binding partner complex(es) to a subject is desired. For example, in certain embodiments, a delivery construct is used to deliver a macromolecule-binding protein complex to a subject, wherein the macromolecule is growth hormone (GH) binding protein and binding partner is growth hormone (GH). GH that is circulated in the blood of a subject is found associated with a binding protein such as GH binding protein. Thus, delivery of a GH-GH binding protein complex mimics the GH found in circulating blood. Further, the GH-GH binding protein complex increases the half-life of GH in the subject. As one skilled in the art is aware, human GH binds human GH biniding protein with a Kₐ that is about 10⁸ M⁻¹.

In certain embodiments, the macromolecule is selected from the group consisting of a nucleic acid, a peptide, a polypeptide, a protein, and a lipid. In further embodiments, the polypeptide is selected from the group consisting of polypeptide hormones, cytokines, chemokines, growth factors, antibodies and clotting factors. In certain embodiments, the macromolecule is IGF-I, IL-2 receptor alpha, IL-18 binding protein, Shc-like protein (Sck) or the SH2 domain of Sck. In specific embodiments, the macromolecule is obtained or derived from the same species as the subject receiving the delivery construct. In preferred embodiments, the macromolecule is a human or humanized macromolecule.

In some embodiments, the carrier construct comprises a macromolecule consisting of multiple subunits. In certain embodiments, the subunits of the macromolecule are separated by a linker of sufficient length to enable the subunits of the macromolecule to fold so that the macromolecule non-covalently binds to its binding partner. In other embodiments, a subunit of the macromolecule is linked to the remainder of the carrier construct and the construct is incubated with one or more other subunits under conditions that permit the subunits to associate and form the macromolecule. In these embodiments, the carrier construct that is used in accordance with the invention comprises the both or all of the subunits of the macromolecule. In specific embodiments, the conditions permit the subunits of a macromolecule to associate in the same manner that they do in nature. In accordance with these embodiments, the binding partner is not a subunit of the macromolecule. For example, in a specific embodiment, the delivery construct is an IL-12 receptor-IL-12 delivery construct. In accordance with this embodiment, the carrier construct may comprise: (i) a receptor-binding domain, (ii) a transcytosis domain, (iii) the beta 1 subunit of IL-12 receptor, and (iv) the beta 2 subunit of IL-12 receptor. Such a carrier construct may be formed by incubating the beta 1 subunit of IL-12 receptor linked to the remainder of the carrier construct with beta 2 subunit of the IL-12 receptor under conditions that permit non-covalently bonds to form between the beta 1 and beta 2 subunits of IL-12 receptor. The carrier construct comprising the non-covalently associated IL-12 receptor subunits is the carrier and the binding partner is, e.g., IL-12.

In certain embodiments, a carrier construct comprises two macromolecules, wherein the second macromolecule is separated from the remainder of the carrier construct by a cleavable linker and cleavage at the cleavable linker separates the second macromolecule from the remainder of said construct. In some embodiments, a carrier construct comprises two macromolecules and two cleavable linkers, wherein the first cleavable linker separates the first macromolecule from the remainder of the construct and the second cleavable linker separates the second macromolecule from the remainder of the construct. The first and second cleavable linkers are, in some embodiments, the same and in other embodiments, different. In a specific embodiment, the second macromolecule is separated from the first macromolecule by a cleavable linker. In certain embodiments, the first macromolecule is a first polypeptide and said second macromolecule is a second polypeptide. In certain embodiments, the first polypeptide and the second polypeptide associate to form a multimer. In certain embodiments, the multimer is a dimer, tetramer, or octamer. In further embodiments, the dimer is an antibody. In vitro studies with polarized epithelial systems representing the gastrointestinal or pulmonary, or other human tissues comprising epithelial cells can be used to assess the capacity (including the efficiency) of linker separation. In specific embodiments, these linkers are 4-8, 4-12, 4-16, 4-20, 8-12, 8-16 or 8-20 amino acids in length for sufficient specificity of an enzyme.

In certain embodiments, two, three, four, five, six, seven, eight, nine, ten, 20, 30, 40, 50, 60, 70, 80, 90, 100, 250, 500, 750, 1,000, 1,500, 2,000, 5,000, or more binding partners may bind to the macromolecule or to binding partners noncovalently linked to the binding partner. For example, the delivery constructs of the present invention can be used to deliver aggregated insulin particles comprising thousands, tens of thousands, hundreds of thousands, millions, or tens of millions of insulin molecules. As one skilled in the art is aware, insulin self-associates to form dimers, hexamers, twelve-mers, twentyfour-mers, fortyeight-mers, etc. See Dodd et al., 1995, Pharm Res. 12:60-68. Association constants for these self-associations have been described for these various states: K₂ (Kₐ for a dimer)=7 ×10⁵ M⁻¹, K₂ (Kₐ for a dimer)=2×10⁹ M⁻¹, K₆ (K^{a} for a hexamer) =7×10⁵ M⁻¹, K1₂ (Kₐ for a twelve-mer)=2×10⁶ M⁻¹, K₂₄ (Kₐ for a twentyfour-mer) =1×10⁶ M⁻¹, K₄₆ (Kₐ for a fourtysix-mer) =4×10¹ M⁻¹, See Chitta et al, 2004, Abstracts, 52^{nd} ASMS Conference on MS and Allied Topics, May 23-27, Nashville, Tenn. Thus, when delivery constructs of the invention comprise either inulin or protamine (to which insulin binds), the delivery construct can be used to deliver insulin complexes as described below.

5.3.1.Receptor Binding Domain

The carrier constructs of the invention generally comprise a receptor binding domain. The receptor binding domain can be any receptor binding domain known to one of skill in the art without limitation to bind to a cell surface receptor that is present on the apical membrane of an epithelial cell. Preferably, the receptor binding domain binds specifically to the cell surface receptor. The receptor binding domain should bind to the cell surface receptor with sufficient affinity to allow endocytosis of the delivery construct.

In certain embodiments, the receptor binding domain can comprise a peptide, a polypeptide, a protein, a lipid, a carbohydrate, or a small organic molecule, or a combination thereof. Examples of each of these molecules that bind to cell surface receptors present on the apical membrane of epithelial cells are well known to those of skill in the art. Suitable peptides or polypeptides include, but are not limited to, bacterial toxin receptor binding domains, such as the receptor binding domains from PE, cholera toxin, botulinum toxin, diptheria toxin, shiga toxin, shiga-like toxin, etc.; antibodies, including monoclonal, polyclonal, and single-chain antibodies, or derivatives thereof, growth factors, such as EGF, IGF-I, IGF-II, IGF-III etc.; cytokines, such as IL-1, IL-2, IL-3, IL-6, etc; chemokines, such as MIP-1a, MIP-1b, MCAF, IL-8, etc.; and other ligands, such as CD4, cell adhesion molecules from the immunoglobulin superfamily, integrins, ligands specific for the IgA receptor, etc. See, e.g., Pastan et al., 1992, Annu. Rev. Biochem. 61:331-54; and U.S. Pat. Nos. 5,668,255, 5,696,237, 5,863,745, 5,965,406, 6,022,950, 6,051,405, 6,251,392, 6,440,419, and 6,488,926. The skilled artisan can select the appropriate receptor binding domain based upon the expression pattern of the receptor to which the receptor binding domain binds.

Lipids suitable for receptor binding domains include, but are not limited to, lipids that themselves bind cell surface receptors, such as sphingosine-1-phosphate, lysophosphatidic acid, sphingosylphosphorylcholine, retinoic acid, etc.; lipoproteins such as apolipoprotein E, apolipoprotein A, etc., and glycolipids such as lipopolysaccharide, etc.; glycosphingolipids such as globotriaosylceramide and galabiosylceramide; and the like. Carbohydrates suitable for receptor binding domains include, but are not limited to, monosaccharides, disaccharides, and polysaccharides that comprise simple sugars such as glucose, fructose, galactose, etc.; and glycoproteins such as mucins, selectins, and the like. Suitable small organic molecules for receptor binding domains include, but are not limited to, vitamins, such as vitamin A, B₁, B₂, B₃, B₆, B₉, B₁₂, C, D, E, and K, amino acids, and other small molecules that are recognized and/or taken up by receptors present on the apical surface of epithelial cells. U.S. Pat. No. 5,807,832 provides an example of such small organic molecule receptor binding domains, vitamin B₁₂.

In certain embodiments, the receptor binding domain can bind to a receptor found on an epithelial cell. In further embodiments, the receptor binding domain can bind to a receptor found on the apical membrane of an epithelial cell. The receptor binding domain can bind to any receptor present on the apical membrane of an epithelial cell by one of skill in the art without limitation. For example, the receptor binding domain can bind to α2-MR, EGFR, or IGFR. An example of a receptor binding domain that can bind to α2-MR is domain Ia of PE. Accordingly, in certain embodiments, the receptor binding domain is domain Ia of PE. In other embodiments, the receptor binding domain is a portion of domain Ia of PE that can bind to α2-MR. Exemplary receptor binding domains that can bind to EGFR include, but are not limited to, EGF and TGFα. Examples of receptor binding domains that can bind to IGFR include, but are not limited to, IGF-I, IGF-II, or IGF-III. Thus, in certain embodiments, the receptor binding domain is EGF, IGF-I, IGF-II, or IGF-III. In other embodiments, the receptor binding domain is a portion of EGF, IGF-I, IGF-II, or IGF-III that can bind to the EGF or IGF receptor.

In certain embodiments, the receptor binding domain binds to a receptor that is highly expressed on the apical membrane of a polarized epithelial cell but is not expressed or expressed at low levels on antigen presenting cells, such as, for example, dendritic cells. Exemplary receptor binding domains that have this kind of expression pattern include, but are not limited to, TGFα, EGF, IGF-I, IGF-I, and IGF-III.

In certain embodiments, the carrier constructs of the invention comprise more than one domain that can function as a receptor binding domain. For example, the carrier construct can comprise PE domain Ia in addition to another receptor binding domain.

The receptor binding domain can be attached to the remainder of the carrier construct by any method or means known by one of skill in the art to be useful for attaching such molecules, without limitation. In certain embodiments, the receptor binding domain is expressed or synthesized together with the remainder of the carrier construct as a fusion protein. Such embodiments are particularly useful when the receptor binding domain and the remainder of the construct are formed from peptides or polypeptides.

In other embodiments, the receptor binding domain is connected with the remainder of the carrier construct with a linker. In yet other embodiments, the receptor binding domain is connected with the remainder of the carrier construct without a linker. Either of these embodiments is useful when the receptor binding domain comprises a peptide, polypeptide, protein, lipid, carbohydrate, nucleic acid, or small organic molecule.

In certain embodiments, the linker can form a covalent bond between the receptor binding domain and the remainder of the carrier construct. In certain embodiments, the covalent bond can be a peptide bond. In other embodiments, the linker can link the receptor binding domain to the remainder of the carrier construct with one or more non-covalent interactions of sufficient affinity. One of skill in the art can readily recognize linkers that interact with each other with sufficient affinity to be useful in the carrier constructs of the invention. For example, biotin can be attached to the receptor binding domain, and streptavidin can be attached to the remainder of the molecule. In certain embodiments, the linker can directly link the receptor binding domain to the remainder of the molecule. In other embodiments, the linker itself comprises two or more molecules that associate in order to link the receptor binding domain to the remainder of the molecule. Exemplary linkers include, but are not limited to, straight or branched-chain carbon linkers, heterocyclic carbon linkers, substituted carbon linkers, unsaturated carbon linkers, aromatic carbon linkers, peptide linkers, etc.

In embodiments where a linker is used to connect the receptor binding domain to the remainder of the carrier construct, the linkers can be attached to the receptor binding domain and/or the remainder of the carrier construct by any means or method known by one of skill in the art without limitation. For example, the linker can be attached to the receptor binding domain and/or the remainder of the carrier construct with an ether, ester, thioether, thioester, amide, imide, disulfide, peptide, or other suitable moiety. The skilled artisan can select the appropriate linker and method for attaching the linker based on the physical and chemical properties of the chosen receptor binding domain and the linker. The linker can be attached to any suitable functional group on the receptor binding domain or the remainder of the molecule. For example, the linker can be attached to sulfhydryl (—S), carboxylic acid (—COOH) or free amine (—NH2) groups, which are available for reaction with a suitable functional group on a linker. These groups can also be used to connect the receptor binding domain directly connected with the remainder of the molecule in the absence of a linker.

Further, the receptor binding domain and/or the remainder of the carrier construct can be derivatized in order to facilitate attachment of a linker to these moieties. For example, such derivatization can be accomplished by attaching suitable derivative such as those available from Pierce Chemical Company, Rockford, Ill. Alternatively, derivatization may involve chemical treatment of the receptor binding domain and/or the remainder of the molecule. For example, glycol cleavage of the sugar moiety of a carbohydrate or glycoprotein receptor binding domain with periodate generates free aldehyde groups. These free aldehyde groups may be reacted with free amine or hydrazine groups on the remainder of the molecule in order to connect these portions of the molecule. See, e.g., U.S. Pat. No. 4,671,958. Further, the skilled artisan can generate free sulfhydryl groups on proteins to provide a reactive moiety for making a disulfide, thioether, thioester, etc. linkage. See, e.g., U.S. Pat. No. 4,659,839.

Any of these methods for attaching a linker to a receptor binding domain and/or the remainder of a carrier construct can also be used to connect a receptor binding domain with the remainder of the carrier construct in the absence of a linker. In such embodiments, the receptor binding domain is coupled with the remainder of the construct using a method suitable for the particular receptor binding domain. Thus, any method suitable for connecting a protein, peptide, polypeptide, nucleic acid, carbohydrate, lipid, or small organic molecule to the remainder of the carrier construct known to one of skill in the art, without limitation, can be used to connect the receptor binding domain to the remainder of the construct. In addition to the methods for attaching a linker to a receptor binding domain or the remainder of a carrier construct, as described above, the receptor binding domain can be connected with the remainder of the construct as described, for example, in U.S. Pat. Nos. 6,673,905; 6,585,973; 6,596,475; 5,856,090; 5,663,312; 5,391,723; 6,171,614; 5,366,958; and 5,614,503.

In certain embodiments, the receptor binding domain can be a monoclonal antibody. In some of these embodiments, the receptor-binding domain is expressed as a fusion protein that comprises an immunoglobulin heavy chain from an immunoglobulin specific for a receptor on a cell to which the chimeric immunogen is intended to bind. The light chain of the immunoglobulin then can be co-expressed with the chimeric immunogen, thereby forming a light chain-heavy chain dimer. In other embodiments, the antibody can be expressed and assembled separately from the remainder of the chimeric immunogen and chemically linked thereto.

5.3.2. Transcytosis Domain

The carrier constructs of the invention also comprise a transcytosis domain. The transcytosis domain can be any transcytosis domain known by one of skill in the art to effect transcytosis of macromolecules that have bound to a cell surface receptor present on the apical membrane of an epithelial cell. In certain embodiments, the transcytosis domain is a transcytosis domain from PE, diptheria toxin, pertussis toxin, cholera toxin, heat-labile E. coli enterotoxin, shiga toxin, or shiga-like toxin. See, for example, U.S. Pat. Nos. 5,965,406, and 6,022,950. In preferred embodiments, the transcytosis domain is domain II of PE.

The transcytosis domain need not, though it may, comprise the entire amino acid sequence of domain II of native PE, which spans residues 253-364 of PE. For example, the transcytosis domain can comprise a portion of PE that spans residues 280-344 of domain II of PE. The amino acids at positions 339 and 343 appear to be necessary for transcytosis. See Siegall et al., 1991, Biochemistry 30:7154-59. Further, conservative or nonconservative substitutions can be made to the amino acid sequence of the transcytosis domain, as long as transcytosis activity is not substantially eliminated. A representative assay that can routinely be used by one of skill in the art to determine whether a transcytosis domain has transcytosis activity is described below.

Without intending to be limited to any particular theory or mechanism of action, the transcytosis domain is believed to permit the trafficking of the carrier construct through a polarized epithelial cell after the construct binds to a receptor present on the apical surface of the polarized epithelial cell. Such trafficking through a polarized epithelial cell is referred to herein as “transcytosis.” This trafficking permits the release of the carrier construct from the basal-lateral membrane of the polarized epithelial cell.

5.3.3. Macromolecules

The delivery constructs of the invention can also comprise a macromolecule. The macromolecule can be attached to the remainder of the carrier construct by any method known by one of skill in the art, without limitation. In certain embodiments, the macromolecule is expressed together with the remainder of the carrier construct as a fusion protein. In such embodiments, the macromolecule can be inserted into or attached to any portion of the carrier construct, so long as the receptor binding domain, the transcytosis domain, and macromolecule retain their respective activities. In some embodiments, the macromolecule is connected with the remainder of the construct with a cleavable linker, or a combination of cleavable linkers, as described below.

In native PE, the Ib loop (domain Ib) spans amino acids 365 to 399, and is structurally characterized by a disulfide bond between two cysteines at positions 372 and 379. This portion of PE is not essential for any known activity of PE, including cell binding, transcytosis, ER retention or ADP ribosylation activity. Accordingly, domain Ib can be deleted entirely, or modified to contain a macromolecule.

Thus, in certain embodiments, the macromolecule can be inserted into domain Ib. If desirable, the macromolecule can be inserted into domain Ib wherein the cysteines at positions 372 and 379 are not cross-linked. This can be accomplished by reducing the disulfide linkage between the cysteines, by deleting the cysteines entirely from the Ib domain, by mutating the cysteines to other residues, such as, for example, serine, or by other similar techniques. Alternatively, the macromolecule can be inserted into the Ib loop between the cysteines at positions 372 and 379. In such embodiments, the disulfide linkage between the cysteines can be used to constrain the macromolecule if desirable. In embodiments where the macromolecule is inserted into domain Ib of PE, or into any other portion of the carrier construct, the macromolecule, in certain embodiments, is flanked by cleavable linkers such that cleavage at the cleavable linkers liberates the macromolecule from the remainder of the construct.

In other embodiments, the macromolecule can be connected with the N-terminal or C-terminal end of a polypeptide portion of the carrier construct. In such embodiments, the method of connection should be designed to avoid interference with other functions of the carrier construct, such as receptor binding or transcytosis. In yet other embodiments, the macromolecule can be connected with a side chain of an amino acid of the carrier construct. In certain embodiments, the macromolecule can be connected with any portion of the carrier construct that does not disrup, e.g., receptor binding, translocation, or binding partner activity. In certain embodiments, the macromolecule is connected with the remainder of the carrier construct with a cleavable linker, as described below. In such embodiments, the macromolecule that non-covalently binds to the binding partner can be connected with the remainder of the carrier construct with one or more cleavable linkers such that cleavage at the cleavable linker(s) separates the macromolecule from the remainder of the carrier construct. It should be noted that, in certain embodiments, the macromolecule of interest can also comprise a short (1-20 amino acids, preferably 1-10 amino acids, and more preferably 1-5 amino acids) leader peptide in addition to the macromolecule of interest that remains attached to the macromolecule following cleavage of the cleavable linker. Preferably, this leader peptide does not affect the activity or immunogenicity of the macromolecule.

In embodiments where the macromolecule is expressed together with another portion of the carrier construct as a fusion protein, the macromolecule can be can be inserted into the carrier construct by any method known to one of skill in the art without limitation. For example, amino acids corresponding to the macromolecule can be inserted directly into the carrier construct, with or without deletion of native amino acid sequences. In certain embodiments, all or part of the Ib domain of PE can be deleted and replaced with the macromolecule. In certain embodiments, the cysteine residues of the Ib loop are deleted so that the macromolecule remains unconstrained. In other embodiments, the cysteine residues of the Ib loop are linked with a disulfide bond and constrain the macromolecule.

In certain embodiments, the macromolecule is any macromolecule that non-covalently to a binding partner(s) of interest. In specific embodiments, the macromolecule of the carrier construct specifically binds to the binding partner(s) of interest. In a specific embodiment, the macromolecule is one that non-covalently binds to one or more of the binding partners recited herein. For example, in certain embodiments, the ratio of macromolecule to binding partner is 2:1, 3:1, 4:1, 5:1 or more.

In certain embodiments, the binding partner-macromolecule interaction has an on-rate sufficient for association and retention during uptake and transport across epithelial cells and an off-rate sufficient for release of the binding partner once the binding partner-macromolecule complex has reached the basolateral surface. In other embodiments, the binding partner-macromolecule interaction has a similar on-rate and/or off-rate as that found in nature.

In certain embodiments, the macromolecule of a carrier construct of the invention has a high association rate constant. In specific embodiments, the macromolecule of a carrier construct of the invention and the binding partner have an association rate constant or kₒₙ rate of about 10⁵ M⁻¹s⁻¹ or more, about 5×10⁵ M⁻¹s⁻¹ or more, about 10⁶ M⁻¹s⁻¹ or more, about 5×10⁶ M⁻¹s⁻¹ or more, about 10⁷ M⁻¹s⁻¹ or more, about 5×10⁷ M⁻¹s⁻¹ or more, about 10⁸ M⁻¹s⁻¹ or more, about 5×10⁸ M⁻¹s⁻¹ or more, or about 1×10⁹ M⁻¹s⁻¹ or more.

In other embodiments, the macromolecule of a carrier construct of the invention and the binding partner have a k_{off} rate of about 5×10⁻¹ s^{31 1} or less, about 10⁻¹ s⁻¹ or less, about 5×10⁻² s⁻¹ or less, about 10⁻² s⁻¹ or less, about 5×10⁻³ s⁻¹ or less, about 10⁻³ s⁻¹ or less, about 5×10⁻⁴ s⁻¹ or less, about 10⁻⁴ s⁻¹ or less, about 5×10⁻⁵ s⁻¹ or less, about 10⁻⁵ s⁻¹ or less, about 5×10⁻⁶ s⁻¹ or less, about 10⁻⁶ s⁻¹ or less, about 5×10⁻⁷ s⁻¹ or less, about 10⁻⁷ s⁻¹ or less, about 5×10⁻⁸ s⁻¹ or less, about 10⁻⁸ s⁻¹ or less, about 5×10⁻⁹ s⁻¹ or less, about 10⁻⁹ s⁻¹ or less, about 5×10⁻¹⁰ s⁻¹ or less, or about 10⁻¹⁰ ⁻¹ or less.

In certain embodiments, the macromolecule of a carrier construct of the invention and the binding partner have an affinity constant or Kₐ (kₒₙ/k_{off}) of about 10² M⁻¹ or more, about 5×10² M⁻¹ or more, about 10³ M⁻¹ or more, about 5×10³ M⁻¹ or more, about 10⁴ M⁻¹ or more, about 5×10⁴ M⁻¹ or more, about 10⁵ M⁻¹ or more, about 5×10⁵ M⁻¹ or more, about 10⁶ M⁻¹ or more, about 5×10⁶ M⁻¹ or more, about 10⁷ M⁻¹ or more, about 5×10⁷ M⁻¹ or more, about 10⁸ M⁻¹ or more, about 5×10⁸ M⁻¹ or more, about 10⁹ M⁻¹ or more, about 5×10⁹ M⁻¹ or more, about 10¹⁰ M⁻¹ or more, about 5×10¹⁰ M⁻¹ or more, about 10¹¹ M⁻¹ or more, about 5×10¹¹ M⁻¹ or more, about 10¹² M⁻¹ or more, about 5×10¹² M⁻¹ or more, about 10¹³ M⁻¹ or more, about 5×10¹³ M⁻¹ or more, about 10¹⁴ M⁻¹ or more, about 5×10¹⁴ M⁻¹ or more, about 10¹⁵ M⁻¹ or more, or about 5×10¹⁵ M⁻¹ or more.

In certain embodiments, the macromolecule of a carrier construct of the invention has a low dissociation constant. In specific embodiments, the macromolecule of a carrier construct of the invention has a high association constant. In certain embodiments, a dissociation constant or K_{d} (k_{off}/kₒₙ) for antibody is about 5×10⁻¹ M or less, about 10⁻¹ M or less, about 5×10² M or less, about 10² M or less, about 5×10⁻³ M or less, about 10⁻³ M or less, about 5×10⁴ M or less, about 10⁴ M or less, about 5×10⁻⁵ M or less, about 10⁻⁵ M or less, about 5×10⁶ M or less, about 10⁶ M or less, about 5×10⁻⁷ M or less, about 10⁻⁷ M or less, about 5×10⁸ M or less, about 10⁸ M or less, about 5×10⁻⁹ M or less, about 10⁻⁹ M or less, about 5×10⁻¹⁰ M or less, or about 10⁻¹⁰ M or less.

In particular embodiments, the macromolecule of the carrier construct is chosen because delivery of a particular macromolecule-binding partner complex(es) to a subject is desired. For example, in certain embodiments, a delivery construct is used to deliver a macromolecule-binding protein complex to a subject, wherein the macromolecule is growth hormone (GH) binding protein and binding partner is growth hormone (GH). GH that is circulated in the blood of a subject is found associated with a binding proteins such as GH binding protein. Thus, the delivery of a GH-GH binding protein complex mimics the GH found in circulating blood. Further, the GH-GH binding protein complex increases the half-life of GH in the subject.

In certain embodiments, the macromolecule can be a peptide, a polypeptide, a protein, a nucleic acid, a carbohydrate, a lipid, a glycoprotein, synthetic organic and inorganic compounds, or any combination thereof. In certain embodiments, the macromolecule can be either soluble or insoluble in water. In certain embodiments, the macromolecule can be a macromolecule that can perform a desirable biological activity when introduced to the bloodstream of the subject. For example, the macromolecule can have receptor binding activity, enzymatic activity, messenger activity (i.e., act as a hormone, cytokine, neurotransmitter, or other signaling molecule), or regulatory activity, or any combination thereof.

In other embodiments, the macromolecule can exert its effects in biological compartments of the subject other than the subject's blood. For example, in certain embodiments, the macromolecule can exert its effects in the lymphatic system. In other embodiments, the macromolecule can exert its effects in an organ or tissue, such as, for example, the subject's liver, heart, lungs, pancreas, kidney, brain, bone marrow, etc. In such embodiments, the macromolecule may or may not be present in the blood, lymph, or other biological fluid at detectable concentrations, yet may still accumulate at sufficient concentrations at its site of action to exert a biological effect.

Further, the macromolecule can be a protein that comprises more than one polypeptide subunit. For example, the protein can be a dimer, trimer, or higher order multimer. In certain embodiments, two or more subunits of the protein can be connected with a covalent bond, such as, for example, a disulfide bond. In other embodiments, the subunits of the protein can be held together with non-covalent interactions. One of skill in the art can routinely identify such proteins and determine whether the subunits are properly associated using, for example, an immunoassay. Exemplary proteins that comprise more than one polypeptide chain include, but are not limited to, antibodies, insulin-like growth factor (IGF)-I receptors, and the like.

Accordingly, in certain embodiments, the macromolecule is a peptide, polypeptide, or protein. In certain embodiments, the macromolecule comprises a peptide or polypeptide that comprises about 5, about 8, about 10, about 12, about 15, about 17, about 20, about 25, about 30, about 40, about 50, or about 60, about 70, about 80, about 90, about 100, about 200, about 400, about 600, about 800, or about 1000 amino acids. In certain embodiments, the macromolecule is a protein that comprises 1, 2, 3, 4, 5, 6, 7, 8, or more polypeptides. In certain embodiments, the peptide, polypeptide, or protein is a molecule that is commonly administered to subjects by injection. Exemplary peptides or polypeptides include, but are not limited to, insulin growth factor binding proteins (IGFBPs; such as IGFBP-1, IGFBP-2, IGFBP-3, IGFBP-4, IGFBP -5, and IGFBP-6), IL-18 binding protein (IL-18BP), fibroblast growth factor binding proteins (FGFBP; such as FGFBP-1), latent transforming growth factor (TGF)-beta binding proteins (such as latent TGF-beta binding proteins-1, -3 and -4), IL-2 receptor alpha, and the like.

In certain embodiments, the macromolecule is a receptor for a growth factor or a cytokine. In other embodiments, the macromolecule is a ligand for a growth factor receptor or a cytokine receptor. In certain embodiments, the macromolecule is a DNA binding protein.

In certain embodiments, the macromolecule is a fragment of a receptor for a growth factor or a cytokine that binds non-covalently to a binding protein. In other embodiments, the macromolecule is a fragment of a ligand for a growth factor receptor or a cytokine receptor that binds non-covalently to a binding protein. In other embodiments, the macromolecule is a fragment of a DNA binding protein that binds non-covalently to a binding protein. In other embodiments, the macromolecule is a domain that binds to multiple binding partners. For example, SH2 domains bind to a number of phosphorylated proteins. In yet other embodiments, the macromolecule is an antigen that binds to an antibody or antibody fragment.

In a specific embodiment, the macromolecule is IGFBP-3 or a fragment thereof that binds to IGF-I or IGF-II. In another specific embodiment, the macromolecule is growth hormone binding protein or a fragment thereof that binds to GH. In another specific embodiment, the macromolecule is IL-2 receptor alpha or a fragment thereof that binds to IL-2. In another specific embodiment, the macromolecule is IL-18BP or a fragment thereof that binds to IL-18. In another specific embodiment, the macromolecule is Shc-like protein (Sck) or the SH2 domain of Sck. In another specific embodiment, the macromolecule is inulin. In another specific embodiment, the macromolecule is protamine. The sequences of all of these macromolecules are well known to those in the art, and attachment of these macromolecules to the carrier constructs is well within the skill of those in the art using standard techniques, as discussed below.

In certain embodiments, the macromolecule non-covalently binds to an antibody (in other words, the macromolecule is an antibody-binding domain). In certain embodiments, an antibody-binding domain of a carrier construct non-covalently binds to a particular type(s), a particular class(es) and/or a particular subclass(es) of an antibody or antibody fragment. In other embodiments, an antibody-binding domain of a carrier construct non-covalently binds to an antibody or antibody fragment specific for a particular antigen. In a specific embodiment, an antibody-binding domain specifically binds to an antibody or an antibody fragment of interest.

In certain embodiments, an antibody-binding domain of a carrier construct non-covalently binds to the Fc region of an antibody. In specific embodiments, an antibody-binding domain of a carrier construct non-covalently binds to the CH2, and/or CH3 region(s) of an antibody. In other embodiments, an antibody-binding domain of a carrier construct non-covalently binds to the CH2, CH3 and hinge regions of an antibody. In yet other embodiments, an antibody-binding domain of a carrier construct non-covalently binds to the CH1 region of an antibody.

In certain embodiments, an antibody-binding domain of a carrier construct comprises a bacterial or bacterial-derived antibody-binding protein, polypeptide or peptide. Non-limiting examples of such antibody-binding domains include Protein A, Protein G, Protein V, Protein L, LAG, Protein LG, Protein AG and antibody-binding fragments thereof. Protein A is produced by Staphylococcus aureus, Protein G is produced by Streptococcus pyogenes, Protein V is produced by Gardnerall vaginalis (see, e.g., U.S. Pat. No. 5,128,451 (which is hereby incorporated by reference) for a description of Protein V), Protein L is produced by Peptostreptococcus magnus, and ZAG is produced by Streptococcus zooepidermicus. Protein LG is a hybrid of Protein L and Protein G (see, e.g., KihIberg et al., 1992, Journal of Biological Chemistry 267: 25583-25588 (which is hereby incorporated by reference) for a description of the hybrid protein). Protein AG is a hybrid of Protein A and Protein G (see, e.g., Sun et al., 1992, Journal of Immunol. Methods 152: 43-48 (which is hereby incorporated by reference) for a description of the hybrid protein). See, e.g., Goward et al., 1993, TIBS 18: 136-140, which is incorporated herein in its entirety, for a discussion about bacterial proteins that bind to cellular receptors, antibodies or antibody fragments.

In certain embodiments, an antibody-binding fragment of a bacterial protein or polypeptide is used as the antibody-binding domain of a carrier construct. For example, in some embodiments, the antibody-binding domain is the Z domain of Protein A. See, e.g., U.S. Pat. No. 6,197,927 and Braisted et al, 1996, PNAS USA 93: 5688-5692 (which are hereby incorporated by reference) for a description of such antibody-binding domains. In other embodiments, the antibody-binding domain is an analog or derivative of a bacterial antibody-binding domain.

In certain embodiments, an antibody-binding domain of a carrier construct is a plant macromolecule that non-covalently binds to an antibody or antibody fragment, such as a plant lectin, or an antibody-binding analog, derivative or fragment thereof. In a specific embodiment, the plant antibody-binding domain is jacalin. Jaculin binds to human IgA1, human IgA2 and human IgD. See, e.g., Aucouturier et al., 1988, J. Immunol. Methods 113(2): 185-91 (which is hereby incorporated by reference) for a description of the antibody binding activity of jaculin.

In certain embodiments, an antibody-binding domain of a carrier construct is a receptor or an analog, derivative or a fragment thereof that binds to the Fc region of an antibody. Preferably, the receptor is from or derived from the same species that is to receive the delivery construct. In a specific embodiment, an antibody-binding domain of a carrier construct is an Fc receptor (FcR) or an analog, derivative or antibody-binding fragment thereof. Non-limiting examples of Fc receptors include FcγRI, FcγRIIA, FcγRIIB, FcγRIIC, FcγRIIIAα, FcγRIIIB, FcεRIα, FcεRIξ, FcγRIIIAξ, and FcRn. See, e.g., Ravetch et al., 1991, Annu. Rev. Immunol. 9: 457-492; Ravetech, 1994, Cell 78: 573-560; Ravetech et al., 2000, Science 290: 84-89; Gerber et al., 2001, Microbes and Infection 131-139; Ravetech, 2001, Annu. Rev. Immunol. 19: 275-290; Genetic et al., 2000, Annu. Rev. Immunol. 18:739-766; U.S. Publication No. 2004/0265321; U.S. Publication No. 2005/0215767; U.S. Publication No. 2004/0185045 (which are hereby incorporated by reference) for descriptions of Fc receptors and fragments thereof.

See, e.g., Table 1 for non-limiting examples of macromolecules. Compounds 1 and 2 listed in Table 1 non-covalently bind to each other. The macromolecule can be compound 1 or compound 2. Alternatively, the macromolecule can be a fragment of either compound 1 or compound 2 that non-covalently binds to compound 2 or compound 1, respectively. Additional examples of macromolecules may be found in: Goodman and Gilman's The Pharmacological Basis of Therapeutics, 9th ed. McGraw-Hill 1996, and Binz et al., 2005, Nature Biotechnology 23: 1257-1268 (in particular, see Table 1 in Binz et al.), which are incorporated herein by reference in their entirety.

TABLE 1

| Compound 1 | Compound 2 |
|---|---|
| Latent TGF-beta binding proteins | TGF-beta |
| IGFBP-1 to IGFBP-6 | IGF-I and IGF-II |
| FGFBP-1 | FGF |
| IL-18BP | IL-18 |
| Retinoic-acid receptors | Tretinoin and alitretinoin |
| Retinoid-X receptors | Alitretinoin |
| IFN receptors | IFN |
| IL-2 receptor alpha | IL-2 |
| Sck | KDR |
| SH2 domain of Sck | KDR |
| IL-9 | IL-9 receptor |
| LFA-3 | CD2 |
| SH3 domain | Different peptides |
| | including Abl-1, Src and |
| | Nef |
| SH2 domain | Phosphorylated peptides |
| GH | GH binding protein |
| VEGFR | VEGF |
| 5G1.1 | Complement (C5) |
| (Ecluizumab) | |
| 5G1.1 | Complement (C5) |
| (Ecluizumab) | |
| 5G1.1 | Complement (C5) |
| (Ecluizumab) | |
| 5G1.1-SC | Complement (C5) |
| (Pexelizumab) | |
| 5G1.1-SC | Complement (C5) |
| (Pexelizumab) | |
| 5G1.1-SC | Complement (C5) |
| (Pexelizumab) | |
| ABX-CBL | CBL |
| (Gavilimomab) | |
| ABX-CBL | CD147 |
| (Gavilimomab) | |
| ABX-IL8 | IL-8 |
| Antegren | VLA-4 |
| (Natalizumab) | |
| Anti-CD11a | CD11a |
| (Efalizumab) | |
| Anti-CD18 | CD18 |
| Anti-LFA1 | CD18 |
| Antova | CD40L |
| Antova | CD40L |
| BTI-322 | CD2 |
| CDP571 | TNF-alpha |
| CDP571 | TNF-alpha |
| CDP850 | E-selectin |
| Corsevin M | Fact VII |
| D2E7 | TNF-alpha |
| (Adalimumab) | |
| Humira | TNF |
| (Adalimumab) | |
| Hu23F2G | CD11/18 |
| (Rovelizumab) | |
| Hu23F2G | CD11/18 |
| (Rovelizumab) | |
| IC14 | CD14 |
| ICM3 | ICAM-3 |
| IDEC-114 | CD80 |
| IDEC-131 | CD40L |
| IDEC-131 | CD40L |
| IDEC-151 | CD4 |
| IDEC-152 | CD23 |
| Infliximab | TNF-alpha |
| Infliximab | TNF-alpha |
| LDP-01 | beta2-integrin |
| LDP-01 | beta2-integrin |
| LDP-02 | Alpha4beta7 |
| MAK-195F | TNF alpha |
| (Afelimomab) | |
| MDX-33 | CD64 (FcR) |
| MDX-CD4 | CD4 |
| MEDI-507 | CD2 |
| (Siplizumab) | |
| MEDI-507 | CD2 |
| (Siplizumab) | |
| OKT4A | CD4 |
| OrthoClone OKT4A | CD4 |
| Remicade | |
| (Infliximab) | |
| Orthoclone/ | CD3 |
| anti-CD3 OKT3 | |
| (Muromonab-CD3) | |
| ReoPro | gpIIbIIIa |
| (Abciximab) | |
| ABX-EGF (Panitimumab) | EGF receptor |
| OvaRex (Oregovemab) | Tumor antigen CA125 |
| BravaRex | Tumor antigen MUC1 |
| Theragyn (pemtumomabytrrium-90) | PEM antigen |
| Therex | PEM antigen |
| Bivatuzumab | CD44 |
| Panorex (Edrecolomab) | 17-1A |
| ReoPro (Abciximab) | Gp IIIb/IIIa |
| ReoPro (Abciximab) | Gp IIIb/IIIa |
| ReoPro (Abciximab) | Gp IIIb/IIIa |
| Bexxar (Tositumomab) | CD20 |
| MAb, idiotypic 105AD7 | Gp72 |
| Anti-EpCAM | Ep-CAM |
| (Catumaxomab) | |
| Herceptin | HER-2 |
| (Trastuzumab) | |
| Herceptin | HER-2 |
| (Trastuzumab) | |
| Rituxan (Rituximab) | CD20 |
| Rituxan (Rituximab) | CD20 |
| Avastin (Bevacizumab) | VEGF |
| AMD Fab | CD18 |
| (Ranibizumab) | |
| E-26 (2^{nd} gen. IgE) | IgE |
| (Omalizumab) | |
| Zevalin (Rituxan + yttrium-90) | CD20 |
| (Ibritumomab tiuxetan) | |
| Cetuximab + innotecan | EGF receptor |
| Cetuximab + cisplatin & radiation | EGF receptor |
| Cetuximab + gemcitabine | EGF receptor |
| Cetuximab + cisplatin + 5FU or Taxol | EGF receptor |
| (paclitaxel) | |
| Cetuximab + carboplatin + paclitaxel | EGF receptor |
| Cetuximab + cisplatin | EGF receptor |
| Cetuximab + radiation | EGF receptor |
| BEC2 + Bacillus Calmette Guerin | mimics ganglioside GD3 |
| BEC2 + Bacillus Calmette Guerin | mimics ganglioside GD3 |
| IMC-1C11 | VEGF-receptor |
| nuC242-DM1 | nuC242 |
| LymphoCide | CD22 |
| (Epratuzumab) | |
| LymphoCide Y-90 | CD22 |
| (Epratuzumab Y-90)) | |
| CEA-Cide | CEA |
| (Labetuzumab) | |
| CEA-Cide Y-90 | CEA |
| (Labetuzumab) | |
| CEA-Scan (Tc-99m-labeled arcitumomab) | CEA |
| CEA-Scan (Tc-99m-labeled arcitumomab) | CEA |
| CEA-Scan (Tc-99m-labeled arcitumomab) | CEA |
| CEA-Scan (Tc-99m-labeled arcitumomab) | CEA |
| LeukoScan (Tc-99m-labeled sulesomab) | CEA |
| LymphoScan (Tc-99m-labeled bectumomab) | CD22 |
| AFP-Scan (Tc-99m-labeled) | AFP |
| HumaRAD-HN (+ yttrium-90) | NA |
| HumaSPECT | NA |
| (Votumumab) | |
| MDX-101 (CTLA-4) | CTLA-4 |
| MDX-210 (her-2 overexpression) | HER-2 |
| MDX-210/MAK | HER-2 |
| Vitaxin | αvβ₃ |
| MAb 425 | EGF receptor |
| IS-IL-2 | Ep-CAM |
| Campath (alemtuzumab) | CD52 |
| CD20-streptavidin (+CD20-streptavidin) | CD20 |
| Avidicin (albumin + NRLU13) | NA |
| Oncolym (+ iodine-131) | HLA-DR 10 beta |
| Cotara (+ iodine-131) | DNA-associated proteins |
| C215 (+ staphylococcal enterotoxin) | NA |
| MAb, lung/kidney cancer | NA |
| Nacolomab tafenatox (C242 + staphylococcal | NA |
| enterotoxin) | |
| Nuvion (Visilizumab) | CD3 |
| SMART M195 | CD33 |
| SMART 1D10 | HLA-DR antigen |
| CEAVac | CEA |
| TriGem | GD2-ganglioside |
| TriAb | MUC-1 |
| CEAVac | CEA |
| TriGem | GD2-ganglioside |
| TriAb | MUC-1 |
| NovoMAb-G2 radiolabeled | NA |
| Monopharm C | SK-1 antigen |
| GlioMAb-H (+ gelonin toxin) | NA |
| Rituxan (Rituximab) | CD20 |
| Rituxan (Rituximab) | CD20 |
| ING-1 | Ep-CAM |

In accordance with the invention, for purposes herein, a species that is a binding partner can be a macromolecule and vice versa. For example, in the case of IL-12 and the IL-12R, binding partner can IL-12 or the IL-12 receptor, and the macromolecule of the carrier construct can be IL-12 receptor or IL-12, respectively.

In certain embodiments, the macromolecule can be selected to not be cleavable by an enzyme present at the basal-lateral membrane of an epithelial cell. For example, the assays described in the examples can be used to routinely test whether such a cleaving enzyme can cleave the macromolecule to be delivered. If so, the macromolecule can be routinely altered to eliminate the offending amino acid sequence recognized by the cleaving enzyme. The altered macromolecule can then be tested to ensure that it retains activity using methods routine in the art.

In certain embodiments, the macromolecule can be inactive or in a less active form when administered, then be activated in the subject. For example, the macromolecule can be a peptide or polypeptide with a masked active site. The peptide or polypeptide can be activated by removing the masking moiety. Such removal can be accomplished by peptidases or proteases in the cases of peptide or polypeptide masking agents. Alternatively, the masking agent can be a chemical moiety that is removed by an enzyme present in the subject. This strategy can be used when it is desirable for the macromolecule to be active in limited circumstances. For example, it may be useful for a macromolecule to be active only in the liver of the subject. In such cases, the macromolecule can be selected to have a masking moiety that can be removed by an enzyme that is present in the liver, but not in other organs or tissues. Exemplary methods and compositions for making and using such masked macromolecules can be found in U.S. Pat. Nos. 6,080,575, 6,265,540, and 6,670,147.

In another example of such embodiments, the macromolecule can be a pro-macromolecule that is activated by a biological activity, for example by processing, present in the subject. Following administration of the pro-macromolecule, it can be activated in the subject by appropriate processing enzymes. It should be noted that many pro-macromolecules exhibit activity similar to that of the fully active molecule. Thus, even if not all of the pro-macromolecule is converted to the fully active form, the pro-molecule can in many cases still exert a desirable biological activity in the subject.

One of skill in the art will appreciate that depending upon the binding partner to be bound (e.g., covalently and/or non-covalently) to a macromolecule, certain macromolecules will be more suitable than others and the skilled artisan will select an appropriate macromolecule accordingly. One of skill in the art will appreciate that depending upon whether the delivery construct is intended to deliver a binding partner or a binding partner-macromolecule complex, the appropriate macromolecule will be selected using techniques and knowledge of the skilled artisan. One of skill in the art will appreciate that the disorder being prevented, treated, managed and/or ameliorated will affect the macromolecule chosen and one of skill in the art will known how to make the appropriate selection. One of skill in the art will also appreciate that the species of the subject being administered a delivery construct of the invention will affect the macromolecule chosen and thus, will select an appropriate macromolecule taking into consideration the species receiving the delivery construct. To minimize an immune response to the macromolecule of the carrier construct, it is preferable to choose a macromolecule that is from or derived from the species receiving the delivery construct. Further, one of skill in the art will appreciate that the affinity of the macromolecule for the binding partner will affect the amount of binding partner or binding partner-macromolecule complex delivered to the subject and the skilled artisan will select a macromolecule with suitable affinity for the binding partner to deliver an sufficient amount of the binding partner or the binding partner-macromolecule complex to the subject to have a prophylactic and/or therapeutic effect.

5.3.4. Cleavable Linkers

In certain embodiments, in the carrier constructs of the invention, the macromolecule to which the binding partner non-covalently binds is connected with the remainder of the carrier construct with one or more cleavable linkers. The number of cleavable linkers present in the construct depends, at least in part, on the location of the macromolecule in relation to the remainder of the carrier construct and the nature of the macromolecule. When the macromolecule is inserted into the carrier construct, the macromolecule can be flanked by cleavable linkers, such that cleavage at both linkers separates the macromolecule. The flanking cleavable linkers can be the same or different from each other. When the macromolecule can be separated from the remainder of the carrier construct with cleavage at a single linker, the carrier constructs can comprise a single cleavable linker. Further, where the macromolecule is, e.g., a dimer or other multimer, each subunit of the macromolecule can be separated from the remainder of the carrier construct and/or the other subunits of the macromolecule by cleavage at the cleavable linker.

The cleavable linkers are generally cleavable by a cleaving enzyme that is present at or near the basal-lateral membrane of an epithelial cell. By selecting the cleavable linker to be cleaved by such enzymes, the macromolecule can be liberated from the remainder of the construct following transcytosis across the mucous membrane and release from the epithelial cell into the cellular matrix on the basal-lateral side of the membrane. Further, cleaving enzymes could be used that are present inside the epithelial cell, such that the cleavable linker is cleaved prior to release of the carrier construct from the basal-lateral membrane, so long as the cleaving enzyme does not cleave the carrier construct before the carrier construct enters the trafficking pathway in the polarized epithelial cell that results in release of the carrier construct and macromolecule from the basal-lateral membrane of the cell.

In certain embodiments, the cleaving enzyme is a peptidase. In other embodiments, the cleaving enzyme is an RNAse or DNAse. In yet other embodiments, the cleaving enzyme can cleave carbohydrates. Preferred peptidases include, but are not limited to, Cathepsin GI, Chymotrypsin I, Elastase I, Subtilisin AI, Subtilisin AII, Thrombin I, and Urokinase I. Table 2 presents these enzymes together with an amino acid sequence that is recognized and cleaved by the particular peptidase.

TABLE 2

Peptidases Present Near Basal-Lateral
Mucous Membranes

Amino Acid Sequence Recognized

| | |
|---|---|
| Peptidase | and Cleaved |

| | |
|---|---|
| Cathepsin GI | Ala-Ala-Pro-Phe (SEQ ID NO.:4) |
| Chymotrypsin I | Gly-Gly-Phe (SEQ ID NO.:5) |
| Elastase I | Ala-Ala-Pro-Val (SEQ ID NO.:6) |
| Subtilisin AI | Gly-Gly-Leu (SEQ ID NO.:7) |
| Subtilisin AII | Ala-Ala-Leu (SEQ ID NO.:8) |
| Thrombin I | Phe-Val-Arg (SEQ ID NO.:9) |
| Urokinase I | Val-Gly-Arg (SEQ ID NO.:10) |

In certain embodiments, the carrier construct can comprise more than one cleavable linker, wherein cleavage at either cleavable linker can separate the macromolecule from the carrier construct. In certain embodiments, the cleavable linker can be selected based on the sequence, in the case of peptide, polypeptide, or protein macromolecules, to avoid the use of cleavable linkers that comprise sequences present in the macromolecule to be delivered. For example, if the macromolecule comprises AAL, the cleavable linker can be selected to be cleaved by an enzyme that does not recognize this sequence.

Further, the cleavable linker preferably exhibits a greater propensity for cleavage than the remainder of the carrier construct. As one skilled in the art is aware, many peptide and polypeptide sequences can be cleaved by peptidases and proteases. In certain embodiments, the cleavable linker is selected to be preferentially cleaved relative to other amino acid sequences present in the carrier construct during administration of the delivery construct. In certain embodiments, the receptor binding domain is substantially (e.g., about 99%, about 95%, about 90%, about 85%, about 80, or about 75%) intact following delivery of the delivery construct to the bloodstream of the subject. In certain embodiments, the translocation domain is substantially (e.g., about 99%, about 95%, about 90%, about 85%, about 80, or about 75%) intact following delivery of the delivery construct to the bloodstream of the subject. In certain embodiments, the macromolecule is substantially (e.g., about 99%, about 95%, about 90%, about 85%, about 80, or about 75%) intact following delivery of the delivery construct to the bloodstream of the subject. In certain embodiments, the cleavable linker is substantially (e.g., about 99%, about 95%, about 90%, about 85%, about 80, or about 75%) cleaved following delivery of the delivery construct to the bloodstream of the subject.

In other embodiments, the cleavable linker is cleaved by a cleaving enzyme found in the plasma of the subject. Any cleaving enzyme known by one of skill in the art to be present in the plasma of the subject can be used to cleave the cleavable linker. Use of such enzymes to cleave the cleavable linkers is less preferred than use of cleaving enzymes found near the basal-lateral membrane of a polarized epithelial cell because it is believed that more efficient cleavage will occur in near the basal-lateral membrane. However, if the skilled artisan determines that cleavage mediated by a plasma enzyme is sufficiently efficient to allow cleavage of a sufficient fraction of the carrier constructs to avoid adverse effects, such plasma cleaving enzymes can be used to cleave the carrier constructs. Accordingly, in certain embodiments, the cleavable linker can be cleaved with an enzyme that is selected from the group consisting of caspase-1, caspase-3, proprotein convertase 1, proprotein convertase 2, proprotein convertase 4, proprotein convertase 4 PACE 4, prolyl oligopeptidase, endothelin cleaving enzyme, dipeptidyl-peptidase IV, signal peptidase, neprilysin, renin, and esterase. See, e.g., U.S. Pat. No. 6,673,574. Table 3 presents these enzymes together with an amino acid sequence(s) recognized by the particular peptidase. The peptidase cleaves a peptide comprising these sequences at the N-terminal side of the amino acid identified with an asterisk.

TABLE 3

Plasma Peptidases

Amino Acid Sequence

| | |
|---|---|
| Peptidase | Recognized and Cleaved |

| | |
|---|---|
| Caspase-1 | Tyr-Val-Ala-Asp-Xaa* |
| | (SEQ ID NO.:11) |
| Caspase-3 | Asp-Xaa-Xaa-Asp-Xaa* |
| | (SEQ ID NO.:12) |
| Proprotein convertase 1 | Arg-(Xaa)ₙ-Arg-Xaa*; |
| | n = 0, 2, 4 or 6 |
| | (SEQ ID NO.:13) |
| Proprotein convertase 2 | Lys-(Xaa)ₙ-Arg-Xaa*; |
| | n = 0, 2, 4, or 6 |
| | (SEQ ID NO.:14) |
| Proprotein convertase 4 | Glp-Arg-Thr-Lys-Arg-Xaa* |
| | (SEQ ID NO.:15) |
| Proprotein convertase 4 | Arg-Val-Arg-Arg-Xaa* |
| PACE 4 | (SEQ ID NO.:16) |
| | Decanoyl-Arg-Val-Arg-Arg- |
| | Xaa* |
| | (SEQ ID NO.:17) |
| Prolyloligopeptidase | Pro-Xaa*-Trp-Val-Pro-Xaa |
| Endothelin cleaving | (SEQ ID NO.:18) |
| enzyme in combination | |
| with dipeptidyl- | |
| peptidase IV | |
| Signal peptidase | Trp-Val*-Ala-Xaa |
| | (SEQ ID NO.:19) |
| Neprilysin in | Xaa-Phe*-Xaa-Xaa |
| combination with | (SEQ ID NO.:20) |
| dipeptidyl-peptidase IV | Xaa-Tyr*-Xaa-Xaa |
| | (SEQ ID NO.:21) |
| | Xaa-Trp*-Xaa-Xaa |
| | (SEQ ID NO.:22) |
| Renin in combination | Asp-Arg-Tyr-Ile-Pro-Phe- |
| with dipeptidyl- | His-Leu*-Leu-(Val, Ala or |
| peptidase IV | Pro)-Tyr-(Ser, Pro, or |
| | Ala) |
| | (SEQ ID NO.:23) |

Thus, in certain more preferred embodiments, the cleavable linker can be any cleavable linker known by one of skill in the art to be cleavable by an enzyme that is present at the basal-lateral membrane of an epithelial cell. In certain embodiments, the cleavable linker comprises a peptide. In other embodiments, the cleavable linker comprises a nucleic acid, such as RNA or DNA. In still other embodiments, the cleavable linker comprises a carbohydrate, such as a disaccharide or a trisaccharide. In certain embodiments, the cleavable linker is a peptide that comprises an amino acid sequence that is selected from the group consisting of Ala-Ala-Pro-Phe (SEQ ID NO.:4), Gly-Gly-Phe (SEQ ID NO.:5), Ala-Ala-Pro-Val (SEQ ID NO.:6), Gly-Gly-Leu (SEQ ID NO.:7), Ala-Ala-Leu (SEQ ID NO.:8), Phe-Val-Arg (SEQ ID NO.:9), Val-Gly-Arg (SEQ ID NO.:10).

Alternatively, in less preferred embodiments, the cleavable linker can be any cleavable linker known by one of skill in the art to be cleavable by an enzyme that is present in the plasma of the subject to whom the delivery construct is administered. In certain embodiments, the cleavable linker comprises a peptide. In other embodiments, the cleavable linker comprises a nucleic acid, such as RNA or DNA. In still other embodiments, the cleavable linker comprises a carbohydrate, such as a disaccharide or a trisaccharide. In certain embodiments, the cleavable linker is a peptide that comprises an amino acid sequence that is selected from the group consisting of amino acid sequences presented in Table 3.

In certain embodiments, the carrier construct comprises more than one cleavable linker. In certain embodiments, cleavage at any of the cleavable linkers will separate the macromolecule to be delivered from the remainder of the carrier construct. In certain embodiments, the carrier construct comprises a cleavable linker cleavable by an enzyme present at the basal-lateral side of a polarized epithelial membrane and a cleavable linkers cleavable by an enzyme that is present in the plasma of the subject to whom the delivery construct is administered.

Further, Tables 4 and 5, below, present results of experiments testing the ability of peptidases to cleave substrates when applied to the basal-lateral or apical surface of a polarized epithelial membrane. The sequences recognized by these enzymes are well-known in the art. Thus, in certain embodiments, the carrier construct comprises a cleavable linker that is cleavable by an enzyme listed in Tables 4 and 5. Preferred peptidases exhibit higher activity on the basolateral side of the membrane. Particularly preferred peptidases exhibit much higher (e.g., 100%, 200%, or more increase in activity relative to the apical side) on the basolateral side. Thus, in certain embodiments, the cleavable linker is cleavable by an enzyme that exhibits 50% higher activity on the basal-lateral side of the membrane than on the apical side of the membrane. In certain embodiments, the cleavable linker is cleavable by an enzyme that exhibits 100% higher activity on the basal-lateral side of the membrane than on the apical side of the membrane. In certain embodiments, the cleavable linker is cleavable by an enzyme that exhibits 200% higher activity on the basal-lateral side of the membrane than on the apical side of the membrane. In certain embodiments, the cleavable linker is cleavable by an enzyme that exhibits 500% higher activity on the basal-lateral side of the membrane than on the apical side of the membrane. In certain embodiments, the cleavable linker is cleavable by an enzyme that exhibits 1,000% higher activity on the basal-lateral side of the membrane than on the apical side of the membrane. In certain embodiments, the cleavable linker is cleavable by an enzyme that exhibits 2,000% higher activity on the basal-lateral side of the membrane than on the apical side of the membrane. In certain embodiments, the cleavable linker is cleavable by an enzyme that exhibits 3,000% higher activity on the basal-lateral side of the membrane than on the apical side of the membrane. In certain embodiments, the cleavable linker is cleavable by an enzyme that exhibits 5,000% higher activity on the basal-lateral side of the membrane than on the apical side of the membrane. In certain embodiments, the cleavable linker is cleavable by an enzyme that exhibits 10,000% higher activity on the basal-lateral side of the membrane than on the apical side of the membrane.

In certain embodiments, the cleavage activity is present in tracheal epithelial cells, but not intestinal epithelial cells. In other embodiments, the cleavage activity is present in intestinal epithelial cells but not tracheal epithelial cells. In certain embodiments, the cleavage activity is present in intestinal epithelial cells and tracheal epithelial cells.

In certain embodiments, the cleavable linker may be cleavable by any enzyme that preferentially cleaves at the basolateral side of an epithelial membrane as compared to the apical side of the membrane. Example 6.1.1.4, below, describes an assay that can be used to assess the activity of such enzymes, while Table 5, appended to the end of this document, provides short names and accession numbers for every known human protease or peptidase. Any cleavage sequence recognized by such proteases or peptidases that preferentially cleaves a test substrate on the basolateral side of an epithelial membrane, or in the plasma, as compared to the apical side of such a membrane can also be used in the methods and compositions of the present invention. In such embodiments, one of skill in the art can readily determine the amino acid sequence recognized by such peptidases or proteases according to standard procedures known in the art or according to the known sequences recognized by the proteases and peptidases.

The examples below provide methods for identifying cleaving enzymes that are present at or near the basal-lateral membrane of a polarized epithelial cell. The skilled artisan can routinely use such methods to identify additional cleaving enzymes and the chemical structure(s) identified and cleaved by such cleaving enzymes. Carrier constructs comprising such cleavable linkers and methods of delivering binding partner-macromolecule complexes using delivery constructs comprising carrier constructs comprising such cleavable linkers are also within the scope of the present invention, whether or not such cleaving enzymes are presented in Table 5.

In other embodiments, the cleavable linker can be a cleavable linker that is cleaved following a change in the environment of the delivery construct. For example, the cleavable linker can be a cleavable linker that is pH sensitive and is cleaved by a change in pH that is experienced when the delivery construct is released from the basal-lateral membrane of a polarized epithelial cell. For instance, the intestinal lumen is strongly alkaline, while plasma is essentially neutral. Thus, a cleavable linker can be a moiety that is cleaved upon a shift from alkaline to neutral pH. The change in the environment of the delivery construct that cleaves the cleavable linker can be any environmental change that that is experienced when the delivery construct is released from the basal-lateral membrane of a polarized epithelial cell known by one of skill in the art, without limitation.

5.4. Binding Partners

Binding partners are the molecules/compounds (including macromolecules) that one desires to deliver to a subject. The binding partner can be any molecule (including macromolecules) that binds to another molecule (e.g., a second macromolecule) that is known to one of skill in the art. In specific embodiments, the binding partner binds non-covalently to another molecule. In other specific embodiments, the binding partner binds covalently to another molecule (e.g., a subunit of a macromolecule). In yet other embodiments, the binding partner binds covalently and non-covalently to another molecule.

In accordance with the invention, for purposes herein, the macromolecule portion of the carrier construct can be a binding partner and vice versa. For example, in the case of IL-12 and the IL-12R, binding partner can IL-12 or the IL-12 receptor, and the macromolecule of the carrier construct can be IL-12 receptor or IL-12, respectively. In some embodiments, the macromolecule and the binding partner can be the same macromolecule in the case of macromolecules that self-associate. For example, in some embodiments, the macromolecule is a first insulin protein and the binding partner is a second insulin protein.

In certain embodiments, the binding partner is a peptide, a polypeptide, a protein, a nucleic acid, a carbohydrate, a lipid, a glycoprotein, synthetic organic compound, inorganic compound, or any combination thereof. In certain embodiments, the binding partner is either soluble or insoluble in water. In certain embodiments, the binding partner performs a desirable biological activity when introduced to the bloodstream of the subject. For example, the binding partner can have receptor binding activity, enzymatic activity, messenger activity (i.e., act as a hormone, cytokine, neurotransmitter, or other signaling molecule), or regulatory activity, or any combination thereof.

In preferred embodiments, the binding partner is useful in the prevention, treatment, management and/or amelioration of disorder or a symptom thereof. In specific embodiments, the binding partner is useful in the prevention, treatment, management and/or amelioration of an autoimmune disorder or an inflammatory disorder or a symptom thereof. In other specific embodiments, the binding partner is useful in the prevention, treatment, management and/or amelioration of a hyperproliferative disorder (e.g., a benign or malignant cancer) or a symptom thereof. In yet other specific embodiments, the binding partner is useful in the prevention, treatment, management and/or amelioration of an infection (e.g., viral, bacteria, and parasitic infection).

In other embodiments, the binding partner that is delivered can exert its effects in biological compartments of the subject other than the subject's blood. For example, in certain embodiments, the binding partner can exert its effects in the lymphatic system. In other embodiments, the binding partner can exert its effects in an organ or tissue, such as, for example, the subject's liver, heart, lungs, pancreas, kidney, brain, bone marrow, etc. In such embodiments, the binding partner may or may not be present in the blood, lymph, or other biological fluid at detectable concentrations, yet may still accumulate at sufficient concentrations at its site of action to exert a biological effect.

Further, the binding partner can be a protein that comprises more than one polypeptide subunit. For example, the protein can be a dimer, trimer, or higher order multimer. In certain embodiments, two or more subunits of the protein can be connected with a covalent bond, such as, for example, a disulfide bond. In other embodiments, the subunits of the protein can be held together with non-covalent interactions. One of skill in the art can routinely identify such proteins and determine whether the subunits are properly associated using, for example, an immunoassay. Exemplary proteins that comprise more than one polypeptide chain that can be delivered with a delivery construct of the invention include, but are not limited to, antibodies, insulin, IGF I, and the like.

Still further, the binding partner can be a plypeptide or protein that binds to itself in addition to or in place of the macromolecule. Such embodiments are particularly useful for delivering a very large number of binding partners to the subject. In such embodiments, the macromolecule to which the binding partner binds can be another molecule of the binding partner. Alternately, the macromolecule can be different from the binding partner. In either case, association of binding partners with binding partners already bound to the macromolecule can result in very large complexes comprisng thousands, millions, billions, or more moleucles of the binding partner. An exemplary binding partner suitable for such embodiments is insulin.

In certain embodiments, the binding partner is a peptide, polypeptide, or protein. In certain embodiments, the binding partner comprises a peptide or polypeptide that comprises about 5, about 8, about 10, about 12, about 15, about 17, about 20, about 25, about 30, about 40, about 50, or about 60, about 70, about 80, about 90, about 100, about 200, about 400, about 600, about 800, or about 1000 amino acids. In certain embodiments, the binding partner is a protein that comprises 1, 2, 3, 4, 5, 6, 7, 8, or more polypeptides. In certain embodiments, the peptide, polypeptide, or protein is a molecule that is commonly administered to subjects by injection. Exemplary peptides or polypeptides include, but are not limited to, IGF-I, IGF-II, IGF-III, EGF, IFN-α, IFN-β, IFNγ, G-CSF, GM-CSF, IL-1, IL-2, IL-3, IL-6, IL-8, IL-12, EPO, growth hormone, clotting factors such as factor VII, vasopressin, calcitonin parathyroid hormone, luteinizing hormone-releasing factor, tissue plasminogen activators, adrenocorticototropin, enkephalin, glucagon-like peptide 1, asparaginase, and the like. In a preferred embodiment, the macromolecule is insulin. In certain preferred embodiments, the polypeptide is growth hormone. In even more preferred embodiments, the polypeptide is human growth hormone. In an equally preferred embodiment, the polypeptide is IFN-α, more preferably IFNα-2b. In an equally preferred embodiment, the polypeptide is insulin or proinsulin. The sequences of all of these binding partners are well known to those in the art, and methods for producing delivery constructs comprising these binding partners is well within the skill of those in the art using standard techniques, as discussed below.

Other examples of binding partners that can be delivered according to the present invention include, but are not limited to, antineoplastic compounds, such as nitrosoureas, e.g., carmustine, lomustine, semustine, strepzotocin; methylhydrazines, e.g., procarbazine, dacarbazine; steroid hormones, e.g., glucocorticoids, estrogens, progestins, androgens, tetrahydrodesoxycaricosterone;. immunoactive compounds such as immunosuppressives, e.g., pyrimethamine, trimethopterin, penicillamine, cyclosporine, azathioprine; and immunostimulants, e.g., levamisole, diethyl dithiocarbamate, enkephalins, endorphins; antimicrobial compounds such as antibiotics, e.g., β-lactam, penicillin, cephalosporins, carbapenims and monobactams, β-lactamase inhibitors, aminoglycosides, macrolides, tetracyclins, spectinomycin; antimalarials, amebicides; antiprotazoals; antifungals, e.g., amphotericin β, antivirals, e.g., acyclovir, idoxuridine, ribavirin, trifluridine, vidarbine, gancyclovir; parasiticides; antihalmintics; radiopharmaceutics; gastrointestinal drugs; hematologic compounds; immunoglobulins; blood clotting proteins, e.g., antihemophilic factor, factor IX complex; anticoagulants, e.g., dicumarol, heparin Na; fibrolysin inhibitors, e.g., tranexamic acid; cardiovascular drugs; peripheral anti-adrenergic drugs; centrally acting antihypertensive drugs, e.g., methyldopa, methyldopa HCl; antihypertensive direct vasodilators, e.g., diazoxide, hydralazine HCl; drugs affecting renin-angiotensin system; peripheral vasodilators, e.g., phentolamine; anti-anginal drugs; cardiac glycosides; vasodilators, e.g., amrinone, milrinone, enoximone, fenoximone, imazodan, sulmazole; antidysrhythmics; calcium entry blockers; drugs affecting blood lipids, e.g., ranitidine, bosentan, rezulin; respiratory drugs; sypathomimetic drugs, e.g., albuterol, bitolterol mesylate, dobutamine HCl, dopamine HCl, ephedrine So, epinephrine, fenfluramine HCl, isoproterenol HCl, methoxamine HCl, norepinephrine bitartrate, phenylephrine HCl, ritodrine HCl; cholinomimetic drugs, e.g., acetylcholine Cl; anticholinesterases, e.g., edrophonium Cl; cholinesterase reactivators; adrenergic blocking drugs, e.g., acebutolol HCl, atenolol, esmolol HCl, labetalol HCl, metoprolol, nadolol, phentolamine mesylate, propanolol HCl; antimuscarinic drugs, e.g., anisotropine methylbromide, atropine SO₄, clinidium Br, glycopyrrolate, ipratropium Br, scopolamine HBr; neuromuscular blocking drugs; depolarizing drugs, e.g., atracurium besylate, hexafluorenium Br, metocurine iodide, succinylcholine Cl, tubocurarine Cl, vecuronium Br; centrally acting muscle relaxants, e.g., baclofen; neurotransmitters and neurotransmitter agents, e.g., acetylcholine, adenosine, adenosine triphosphate; amino acid neurotransmitters, e.g., excitatory amino acids, GABA, glycine; biogenic amine neurotransmitters, e.g., dopamine, epinephrine, histamine, norepinephrine, octopamine, serotonin, tyramine; neuropeptides, nitric oxide, K⁺ channel toxins; antiparkinson drugs, e.g., amaltidine HCl, benztropine mesylate, carbidopa; diuretic drugs, e.g., dichlorphenamide, methazolamide, bendroflumethiazide, polythiazide; antimigraine drugs, e.g, carboprost tromethamine mesylate, methysergide maleate.

Still other examples of binding partners that can be delivered according to the present invention include, but are not limited to, hormones such as pituitary hormones, e.g., chorionic gonadotropin, cosyntropin, menotropins, somatotropin, iorticotropin, protirelin, thyrotropin, vasopressin, lypressin; adrenal hormones, e.g., beclomethasone dipropionate, betamethasone, dexamethasone, triamcinolone; pancreatic hormones, e.g., glucagon, insulin; parathyroid hormone, e.g., dihydrochysterol; thyroid hormones, e.g., calcitonin etidronate disodium, levothyroxine Na, liothyronine Na, liotrix, thyroglobulin, teriparatide acetate; antithyroid drugs; estrogenic hormones; progestins and antagonists; hormonal contraceptives; testicular hormones; gastrointestinal hormones, e.g., cholecystokinin, enteroglycan, galanin, gastric inhibitory polypeptide, epidermal growth factor-urogastrone, gastric inhibitory polypeptide, gastrin-releasing peptide, gastrins, pentagastrin, tetragastrin, motilin, peptide YY, secretin, vasoactive intestinal peptide, sincalide.

Still other examples of binding partners that can be delivered according to the present invention include, but are not limited to, enzymes such as hyaluronidase, streptokinase, tissue plasminogen activator, urokinase, PGE-adenosine deaminase; intravenous anesthetics such as droperidol, etomidate, fetanyl citrate/droperidol, hexobarbital, ketamine HCl, methohexital Na, thiamylal Na, thiopental Na; antiepileptics, e.g., carbamazepine, clonazepam, divalproex Na, ethosuximide, mephenytoin, paramethadione, phenytoin, primidone.

Still other examples of binding partners that can be delivered according to the present invention include, but are not limited to, peptides and proteins such as ankyrins, arrestins, bacterial membrane proteins, clathrin, connexins, dystrophin, endothelin receptor, spectrin, selectin, cytokines; chemokines; growth factors, insulin, erythropoietin (EPO), tumor necrosis factor (TNF), neuropeptides, neuropeptide Y, neurotensin, transforming growth factor α, transforming growth factor β, interferon (IFN); hormones, growth inhibitors, e.g., genistein, steroids etc; glycoproteins, e.g., ABC transporters, platelet glycoproteins, GPIb-IX complex, GPIIb-IIIa complex, vitronectin, thrombomodulin, CD4, CD55, CD58, CD59, CD44, lymphocye function-associated antigen, intercellular adhesion molecule, vascular cell adhesion molecule, Thy-1, antiporters, CA-15-3 antigen, fibronectins, laminin, myelin-associated glycoprotein, GAP, GAP-43.

Yet other examples of binding partners that can be delivered according to the present invention include, but are not limited to, cytokines and cytokine receptors such as Interleukin-1(IL-1), IL-2, IL-3, IL-4, IL-5, IL-6, IL-7, IL-8, IL-9, IL-10, IL11, IL-12, IL-13, IL-14, IL-15, IL-16, IL-17, IL-18, IL-1 receptor, IL-2 receptor, IL-3 receptor, IL-4 receptor, IL-5 receptor, IL-6 receptor, IL-7 receptor, IL-8 receptor, IL-9 receptor, IL-10 receptor, IL-11 receptor, IL-12 receptor, IL-13 receptor, IL-14 receptor, IL-15 receptor, IL-16 receptor, IL-17 receptor, IL-18 receptor, lymphokine inhibitory factor, macrophage colony stimulating factor, platelet derived growth factor, stem cell factor, tumor growth factor β, tumor necrosis factor, lymphotoxin, Fas, granulocyte colony stimulating factor, granulocyte macrophage colony stimulating factor, interferon α, interferon β, and interferon γ.

Still other examples of binding partners that can be delivered according to the present invention include, but are not limited to, growth factors and protein hormones such as erythropoietin, angiogenin, hepatocyte growth factor, fibroblast growth factor, keratinocyte growth factor, nerve growth factor, tumor growth factor α, thrombopoietin, thyroid stimulating factor, thyroid releasing hormone, neurotrophin, epidermal growth factor, VEGF, ciliary neurotrophic factor, LDL, somatomedin, insulin growth factor, insulin-like growth factor I and II; chemokines such as ENA-78, ELC, GRO-α, GRO-β, GRO-γ, HRG, LIF, IP-10, MCP-1, MCP-2, MCP-3, MCP-4, MIP-1α, MIP-1β, MIG, MDC, NT-3, NT-4, SCF, LIF, leptin, RANTES, lymphotactin, eotaxin-1, eotaxin-2, TARC, TECK, WAP-1, WAP-2, GCP-1, GCP-2; α-chemokine receptors, e.g., CXCR1, CXCR2, CXCR3, CXCR4, CXCR5, CXCR6, CXCR7; and β-chemokine receptors, e.g., CCR1, CCR2, CCR3, CCR4, CCR5, CCR6, CCR7.

Yet other examples of binding partners that can be delivered according to the present invention include, but are not limited to, chemotherapeutics, such as chemotherapy or anti-tumor agents which are effective against various types of human cancers, including leukemia, lymphomas, carcinomas, sarcomas, myelomas etc., such as, for example, doxorubicin, mitomycin, cisplatin, daunorubicin, bleomycin, actinomycin D, and neocarzinostatin.

Still other examples of binding partners that can be delivered according to the present invention include, but are not limited to, antibodies such as anti-cluster of differentiation antigen CD-1 through CD-166 and the ligands or counter receptors for these molecules; anti-cytokine antibodies, e.g., anti-IL-1 through anti-IL-18 and the receptors for these molecules; anti-immune receptor antibodies; antibodies against T cell receptors, major histocompatibility complexes I and II, B cell receptors, selectin killer inhibitory receptors, killer activating receptors, OX-40, MadCAM-1, Gly-CAM1, integrins, cadherens, sialoadherens, Fas, CTLA-4, Fc γ-receptors, Fc α-receptors, Fc ε-receptors, Fc μ-receptors, and their ligands; anti-metalloproteinase antibodies, e.g., antibodies specific for collagenase, MMP-1 through MMP-8, TIMP-1, TIMP-2; anti-cell lysis/proinflammatory molecules, e.g., perforin, complement components, prostanoids, nitron oxide, thromboxanes; and anti-adhesion molecules, e.g., carcioembryonic antigens, lamins, fibronectins.

Yet other examples of binding partners that can be delivered according to the present invention include, but are not limited to, antiviral agents such as reverse transcriptase inhibitors and nucleoside analogs, e.g., ddI, ddC, 3TC, ddA, AZT; protease inhibitors, e.g., Invirase, ABT-538; and inhibitors of in RNA processing, e.g., ribavirin.

Further, specific examples of binding partners that can be delivered with the delivery constructs of the present invention Capoten, Monopril, Pravachol, Avapro, Plavix, Cefzil, Duricef/Ultracef, Azactam, Videx, Zerit, Maxipime, VePesid, Paraplatin, Platinol, Taxol, UFT, Buspar, Serzone, Stadol NS, Estrace, Glucophage (Bristol-Myers Squibb); Ceclor, Lorabid, Dynabac, Prozac, Darvon, Permax, Zyprexa, Humalog, Axid, Gemzar, Evista (Eli Lily); Vasotec/Vaseretic, Mevacor, Zocor, Prinivil/Prinizide, Plendil, Cozaar/Hyzaar, Pepcid, Prilosec, Primaxin, Noroxin, Recombivax HB, Varivax, Timoptic/XE, Trusopt, Proscar, Fosamax, Sinemet, Crixivan, Propecia, Vioxx, Singulair, Maxalt, Ivermectin (Merck & Co.); Diflucan, Unasyn, Sulperazon, Zithromax, Trovan, Procardia XL, Cardura, Norvasc, Dofetilide, Feldene, Zoloft, Zeldox, Glucotrol XL, Zyrtec, Eletriptan, Viagra, Droloxifene, Aricept, Lipitor (Pfizer); Vantin, Rescriptor, Vistide, Genotropin, Micronase/Glyn./Glyb., Fragmin, Total Medrol, Xanax/alprazolam, Sermion, Halcion/triazolam, Freedox, Dostinex, Edronax, Mirapex, Pharmorubicin, Adriamycin, Camptosar, Remisar, Depo-Provera, Caverject, Detrusitol, Estring, Healon, Xalatan, Rogaine (Pharmacia & Upjohn); Lopid, Accrupil, Dilantin, Cognex, Neurontin, Loestrin, Dilzem, Fempatch, Estrostep, Rezulin, Lipitor, Omnicef, FemHRT, Suramin, and Clinafloxacin (Warner Lambert).

See, e.g., Table 1, supra, for non-limiting examples of binding partners. Compounds 1 and 2 listed in Table 1 non-covalently bind to each other. The binding partner can be compound 1 or compound 2. Alternatively, the binding partner can be a fragment of either compound 1 or compound 2 that non-covalently binds to compound 2 or compound 1, respectively. In a preferred embodiment, the binding partner is a compound 1 listed in Table 1, supra. Additional examples of binding partners may be found in: Goodman and Gilman's The Pharmacological Basis of Therapeutics, 9th ed. McGraw-Hill 1996, incorporated herein by reference in its entirety.

In certain embodiments, the binding partner can be inactive or in a less active form when administered, then be activated in the subject. For example, the binding partner can be a peptide or polypeptide with a masked active site. The peptide or polypeptide can be activated by removing the masking moiety. Such removal can be accomplished by peptidases or proteases in the cases of peptide or polypeptide masking agents. Alternatively, the masking agent can be a chemical moiety that is removed by an enzyme present in the subject. This strategy can be used when it is desirable for the binding partner to be active in limited circumstances. For example, it may be useful for a binding partner to be active only in the liver of the subject. In such cases, the binding partner can be selected to have a masking moiety that can be removed by an enzyme that is present in the liver, but not in other organs or tissues. Exemplary methods and compositions for making and using such masked binding partner can be found in U.S. Pat. Nos. 6,080,575, 6,265,540, and 6,670,147.

In another example of such embodiments, the binding partner can be a pro-macromolecule that is activated by a biological activity, for example by processing, present in the subject. For example, the exemplary binding partner proinsulin can be delivered with a delivery construct of the present invention. Following delivery of the pro-macromolecule, it can be activated in the subject by appropriate processing enzymes. While it is believed that proinsulin is processed by enzymes (the endoproteases PC2 and PC3) present in highest concentration in secretory granules of pancreatic beta-cells, it is also believed that such enzyme are present in sufficient concentration in other compartments to permit activation of the pro-macromolecule into its fully active form. Further, it should be noted that many pro-binding partners, including, for example, proinsulin, also exhibit activity similar to that of the fully active molecule. See, for example, Desbuquois et al., 2003, Endocrinology 12:5308-5321. Thus, even if conversion of the pro-binding partner to the fully active form is incomplete, the pro-molecule can in many cases still exert a desirable biological activity in the subject.

One of skill in the art will appreciate that the disorder being prevented, treated, managed and/or ameliorated will affect the binding partner chosen and one of skill in the art will known how to make the appropriate selection. One of skill in the art will also appreciate that the species of the subject being administered a delivery construct of the invention will affect the binding partner chosen and thus, will select an appropriate binding partner taking into consideration the species receiving the delivery construct. To minimize an immune response to the binding partner, it is preferable to choose a binding partner that is from or derived from the species receiving the delivery construct.

5.5. Methods for Delivering a Macromolecule

In another aspect, the invention provides methods for local or systemic delivery of a binding partner or a binding partner-macromolecule complex to a subject. These methods generally comprise administering a delivery construct of the invention to a mucous membrane of the subject to whom the binding partner or the binding partner-macromolecule complex is delivered. The delivery construct is typically administered in the form of a pharmaceutical composition, as described below.

Thus, in certain aspects, the invention provides a method for delivering a binding partner or a binding partner-macromolecule complex to a subject. In certain embodiments, the methods comprise contacting an apical surface of a polarized epithelial cell of the subject with a delivery construct. In certain embodiments, the delivery construct comprises a carrier construct non-covalently bound to a binding partner, wherein the carrier construct comprises a receptor binding domain, a transcytosis domain, a macromolecule to which the binding partner non-covalently binds and, optionally, a cleavable linker. In other embodiments, the delivery construct comprises a carrier construct covalently bound to a binding partner, wherein the binding partner is a subunit of a macromolecule and the carrier construct comprises a receptor-binding domain, a transcytosis domain, a second subunit of the macromolecule to which the binding partner covalently binds and, optionally, a cleavable linker. In other embodiments, the delivery construct comprises a carrier construct non-covalently and covalently bound to a binding partner, wherein the binding partner is a subunit of a macromolecule and the carrier construct comprises a receptor-binding domain, a transcytosis domain, a second subunit of the macromolecule to which the binding partner non-covalently and covalently binds and, optionally, a cleavable linker.

The invention also provides methods for local or systemic delivery of a binding partner or a binding partner-macromolecule complex to a subject, the methods comprising administering concurrently a carrier construct of the invention and a binding partner of the invention to a mucous membrane of the subject to whom the binding partner or the binding partner-macromolecule complex is delivered. In this context, the term concurrently refers to the administration of the carrier construct and the binding partner within about 1 minute, about 2 minutes, about 5 minutes, about 10minutes, about 15 minutes, about 30 minutes, about 60 minutes, about 90 minutes, about 2 hours, about 4 hours, about 6 hours, about 10 hours, about 12 hours or within about 24 hours of each other. In a preferred embodiment, the carrier construct and the binding partner are administered to each other within one doctor's visit. The carrier construct and binding partner are typically administered in the form of a pharmaceutical composition, as described below. Any method of administration known to one skill in the art can be used to administer a carrier construct and a binding partner, see, e.g., those in Section 5.5.1, infra.

The transcytosis domain of the carrier construct can transcytose the binding partner or the binding partner-macromolecule complex to and through the basal-lateral membrane of said epithelial cell. The cleavable linker of the carrier construct can be cleaved by an enzyme that is present at a basal-lateral membrane of a polarized epithelial cell of the subject or in the plasma of the subject. Cleavage at the cleavable linker separates the macromolecule from the remainder of the carrier construct, thereby delivering the binding partner-macromolecule complex to the subject.

In certain embodiments, the enzyme that is present at or near a basal-lateral membrane of a polarized epithelial cell is selected from the group consisting of Cathepsin GI, Chymotrypsin I, Elastase I, Subtilisin AI, Subtilisin AII, Thrombin I, and Urokinase I. In certain embodiments, the cleavable linker comprises an amino acid sequence that is selected from the group consisting of Ala-Ala-Pro-Phe (SEQ ID NO.:4), Gly-Gly-Phe (SEQ ID NO.:5), Ala-Ala-Pro-Val (SEQ ID NO.:6), Gly-Gly-Leu (SEQ ID NO.:7), Ala-Ala-Leu (SEQ ID NO.:8), Phe-Val-Arg (SEQ ID NO.:9), Val-Gly-Arg (SEQ ID NO.:10).

In certain embodiments, the receptor binding domain of the carrier construct is selected from the group consisting of receptor binding domains from Pseudomonas exotoxin A, cholera toxin, diptheria toxin, shiga toxin, or shiga-like toxin; monoclonal antibodies; polyclonal antibodies; single-chain antibodies; TGF α; EGF; IGF-I; IGF-II; IGF-III; IL-1; IL-2; IL-3; IL-6; MIP-1a; MIP-1b; MCAF; and IL-8. In certain embodiments, the receptor binding domain binds to a cell surface receptor selected from the group consisting of α2-macroglobulin receptor, EGFR, IGFR, transferrin receptor, chemokine receptor, CD25, CD11B, CD11C, CD80, CD86, TNFα receptor, TOLL receptor, M-CSF receptor, GM-CSF receptor, scavenger receptor, and VEGF receptor.

In certain embodiments, the transcytosis domain is selected from the group consisting of transcytosis domains from Pseudomonas exotoxin A, diptheria toxin, pertussis toxin, cholera toxin, heat-labile E. coli enterotoxin, shiga toxin, and shiga-like toxin.

In certain embodiments, the macromolecule is selected from the group of a nucleic acid, a peptide, a polypeptide, a protein, and a lipid. In further embodiments, the polypeptide is selected from the group consisting of polypeptide hormones, cytokines, chemokines, growth factors, antibodies and clotting factors. In certain embodiments, the macromolecule is IGF-I, IL-2 receptor alpha, IL-18 binding protein, Shc-like protein (Sck) or the SH2 of Sck. In specific embodiments, the macromolecule is obtained or derived from the same species as the subject receiving the delivery construct. In preferred embodiments, the macromolecule is a human or humanized macromolecule.

Binding partners are the molecules/compounds (including macromolecules) that one desires to deliver to a subject. The binding partner can be any molecule (including macromolecules) that binds (e.g., covalently and/or non-covalently) to another molecule (e.g., a second macromolecule) that is known to one of skill in the art. In certain embodiments, the binding partner is a peptide, a polypeptide, a protein, a nucleic acid, a carbohydrate, a lipid, a glycoprotein, synthetic organic compound, inorganic compound, or any combination thereof. In specific embodiments, the binding partner is obtained or derived from the same species as the subject receiving the delivery construct. In preferred embodiments, the binding partner is a human or humanized macromolecule.

In certain embodiments, the invention provides a method for delivering a binding partner or a binding partner-macromolecule complex to the bloodstream of a subject that results in at least about 30% bioavailability of the binding partner or the binding partner-macromolecule complex, comprising administering a delivery construct to the subject, thereby delivering at least about 30% of the total binding partner or the total binding partner-macromolecule complex administered to the blood of the subject in a bioavailable form of the macromolecule. In certain embodiments, at least about 10% of the total binding partner or the total binding partner-macromolecule complex administered is bioavailable to the subject. In certain embodiments, at least about 15% of the total binding partner or the total binding partner-macromolecule complex administered is bioavailable to the subject. In certain embodiments, at least about 20% of the total binding partner or the total binding partner-macromolecule complex administered is bioavailable to the subject. In certain embodiments, at least about 25% of the total binding partner or the total binding partner-macromolecule complex administered is bioavailable to the subject. In certain embodiments, at least about 35% of the total binding partner or the total binding partner-macromolecule complex administered is bioavailable to the subject. In certain embodiments, at least about 40% of the total binding partner or the total binding partner-macromolecule complex administered is bioavailable to the subject. In certain embodiments, at least about 45% of the total binding partner or the total binding partner-macromolecule complex administered is bioavailable to the subject. In certain embodiments, at least about 50% of the total binding partner or the total binding partner-macromolecule complex administered is bioavailable to the subject.

In certain embodiments, at least about 55% of the total binding partner or the total binding partner-macromolecule complex administered is bioavailable to the subject. In certain embodiments, at least about 60% of the total binding partner or the total binding partner-macromolecule complex administered is bioavailable to the subject. In certain embodiments, at least about 65% of the total binding partner or the total binding partner-macromolecule complex administered is bioavailable to the subject. In certain embodiments, at least about 70% of the total binding partner or the total binding partner-macromolecule complex administered is bioavailable to the subject. In certain embodiments, at least about 75% of the total binding partner or the total binding partner-macromolecule complex administered is bioavailable to the subject. In certain embodiments, at least about 80% of the total binding partner or the total binding partner-macromolecule complex administered is bioavailable to the subject. In certain embodiments, at least about 85% of the total binding partner or the total binding partner-macromolecule complex administered is bioavailable to the subject. In certain embodiments, at least about 90% of the total binding partner or the total binding partner-macromolecule complex administered is bioavailable to the subject. In certain embodiments, at least about 95% of the total binding partner or the total binding partner-macromolecule complex administered is bioavailable to the subject. In certain embodiments, the percentage of bioavailability of the binding partner or the binding partner-macromolecule complex is determined by comparing the amount of binding partner or binding partner-macromolecule complex present in a subject's blood following administration of a delivery construct comprising the binding partner or the binding partner-macromolecule complex to the amount of binding partner or binding partner-macromolecule complex present in a subject's blood following administration of the macromolecule through another route of administration. In certain embodiments, the other route of administration is injection, e.g., subcutaneous injection, intravenous injection, intra-arterial injection, etc. In other embodiments, the percentage of bioavailability of the binding partner or the binding partner-macromolecule complex is determined by comparing the amount of binding partner or binding partner-macromolecule complex present in a subject's blood following administration of a delivery construct comprising the binding partner or the binding partner-macromolecule complex to the total amount of binding partner or binding partner-macromolecule complex administered as part of the delivery construct.

In certain embodiments, peak plasma concentrations of the delivered binding partner or binding partner-macromolecule complex in the subject are achieved about 10 minutes after administration. In certain embodiments, peak plasma concentrations of the delivered binding partner or binding partner-macromolecule complex in the subject are achieved about 15 minutes after administration. In certain embodiments, peak plasma concentrations of the delivered binding partner or binding partner-macromolecule complex in the subject are achieved about 5 minutes after administration. In certain embodiments, peak plasma concentrations of the delivered binding partner or binding partner-macromolecule complex in the subject are achieved about 20 minutes after administration. In certain embodiments, peak plasma concentrations of the delivered binding partner or binding partner-macromolecule complex in the subject are achieved about 25 minutes after administration. In certain embodiments, peak plasma concentrations of the delivered binding partner or binding partner-macromolecule complex in the subject are achieved about 30 minutes after administration. In certain embodiments, peak plasma concentrations of the delivered binding partner or binding partner-macromolecule complex in the subject are achieved about 35 minutes after administration. In certain embodiments, peak plasma concentrations of the delivered binding partner or binding partner-macromolecule complex in the subject are achieved about 40 minutes after administration. In certain embodiments, peak plasma concentrations of the delivered binding partner or binding partner-macromolecule complex in the subject are achieved about 45 minutes after administration. In certain embodiments, peak plasma concentrations of the delivered binding partner or binding partner-macromolecule complex in the subject are achieved about 50 minutes after administration. In certain embodiments, peak plasma concentrations of the delivered binding partner or binding partner-macromolecule complex in the subject are achieved about 55 minutes after administration. In certain embodiments, peak plasma concentrations of the delivered binding partner or binding partner-macromolecule complex in the subject are achieved about 60 minutes after administration. In certain embodiments, peak plasma concentrations of the delivered binding partner or binding partner-macromolecule complex in the subject are achieved about 90 minutes after administration. In certain embodiments, peak plasma concentrations of the delivered binding partner or binding partner-macromolecule complex in the subject are achieved about 120 minutes after administration.

In certain embodiments, the peak plasma concentration of the delivered binding partner or binding partner-macromolecule complex is between about 0.01 ng/ml plasma and about 10 μg/ml plasma. In certain embodiments, the peak plasma concentration of the delivered binding partner or binding partner-macromolecule complex is between about 0.01 ng/ml plasma and about 1 μg/ml plasma. In certain embodiments, the peak plasma concentration of the delivered binding partner or binding partner-macromolecule complex is between about 0.01 ng/ml plasma and about 0.1 μg/ml plasma. In certain embodiments, the peak plasma concentration of the delivered binding partner or binding partner-macromolecule complex is between about 0.01 ng/ml plasma and about 10 ng/ml plasma. In certain embodiments, the peak plasma concentration of the delivered binding partner or binding partner-macromolecule complex is between about 1 ng/ml plasma and about 10 μg/ml plasma. In certain embodiments, the peak plasma concentration of the delivered binding partner or binding partner-macromolecule complex is between about 1 ng/ml plasma and about 1 μg/ml plasma. In certain embodiments, the peak plasma concentration of the delivered binding partner or binding partner-macromolecule complex is between about 1 ng/ml plasma and about 0.5 μg/ml plasma. In certain embodiments, the peak plasma concentration of the delivered binding partner or binding partner-macromolecule complex is between about 1 ng/ml plasma and about 0.1 μg/ml plasma. In certain embodiments, the peak plasma concentration of the delivered binding partner or binding partner-macromolecule complex is between about 10 ng/ml plasma and about 1 μg/ml plasma. In certain embodiments, the peak plasma concentration of the delivered binding partner or binding partner-macromolecule complex is between about 10 ng/ml plasma and about 0.5 μg/ml plasma.

In certain embodiments, the peak plasma concentration of the delivered binding partner or binding partner-macromolecule complex is at least about 10 μg/ml plasma. In certain embodiments, the peak plasma concentration of the delivered binding partner or binding partner-macromolecule complex is at least about 5 μg/ml plasma. In certain embodiments, the peak plasma concentration of the delivered binding partner or binding partner-macromolecule complex is at least about 1 μg/ml plasma. In certain embodiments, the peak plasma concentration of the delivered binding partner or binding partner-macromolecule complex is at least about 500 ng/ml plasma. In certain embodiments, the peak plasma concentration of the delivered binding partner or binding partner-macromolecule complex is at least about 250 ng/ml plasma. In certain embodiments, the peak plasma concentration of the delivered binding partner or binding partner-macromolecule complex is at least about 100 ng/ml plasma. In certain embodiments, the peak plasma concentration of the delivered binding partner or binding partner-macromolecule complex is at least about 50 ng/ml plasma. In certain embodiments, the peak plasma concentration of the delivered binding partner or binding partner-macromolecule complex is at least about 10 ng/ml plasma. In certain embodiments, the peak plasma concentration of the delivered binding partner or binding partner-macromolecule complex is at least about 5 ng/ml plasma. In certain embodiments, the peak plasma concentration of the delivered binding partner or binding partner-macromolecule complex is at least about 1 ng/ml plasma. In certain embodiments, the peak plasma concentration of the delivered macromolecule is at least about 0.1 ng/ml plasma.

Moreover, without intending to be bound to any particular theory or mechanism of action, it is believed that oral administration of a delivery construct can deliver a higher effective concentration of the delivered binding partner or binding partner-macromolecule complex to the liver of the subject than is observed in the subject's plasma. “Effective concentration,” in this context, refers to the concentration experienced by targets of the binding partner or binding partner-macromolecule complex and can be determined by monitoring and/or quantifying downstream effects of binding partner-target interactions or binding partner-macromolecule complex-target interactions. While still not bound to any particular theory, it is believed that oral administration of the delivery construct results in absorption of the delivery construct through polarized epithelial cells of the digestive mucosa, e.g., the intestinal mucosa, followed by cleavage of the construct and release of the macromolecule at the basolateral side of the mucous membrane. As one of skill in the art will recognize, the blood at the basolateral membrane of such digestive mucosa is carried from this location to the liver via the portal venous system. Thus, when the binding partner or binding partner-macromolecule complex exerts a biological activity in the liver, such as, for example, activities mediated by growth hormone, insulin, IGF-I, etc. binding to their cognate receptors, the binding partner or binding partner-macromolecule complex is believed to exert an effect in excess of what would be expected based on the plasma concentrations observed in the subject. Accordingly, in certain embodiments, the invention provides a method of administering a binding partner to a subject that comprises orally administering a delivery construct comprising the binding partner, wherein the binding partner is delivered to the subject's liver at a higher effective concentration than observed in the subject's plasma. In other embodiments, the invention provides a method of administering a binding partner-macromolecule complex to a subject that comprises orally administering a delivery construct, wherein the binding partner-macromolecule complex is delivered to the subject's liver at a higher effective concentration than observed in the subject's plasma.

In certain embodiments, the epithelial cell is selected from the group consisting of nasal epithelial cells, oral epithelial cells, intestinal epithelial cells, rectal epithelial cells, vaginal epithelial cells, and pulmonary epithelial cells.

In certain embodiments, the subject is a mammal. In further embodiments, the subject is a rodent, a lagomorph, or a primate. In yet further embodiments, the rodent is a mouse or rat. In other embodiments, the lagomorph is a rabbit. In still other embodiments, the primate is a human, monkey, or ape. In a preferred embodiment, the subject is a human.

In another aspect, the invention provides a method for delivering a binding partner or binding partner-macromolecule complex to the bloodstream of a subject that induces a lower titer of antibodies against the binding partner or the binding partner-macromolecule complex than other routes of administration. Without intending to be bound by any particular theory or mechanism of action, it is believed that entry of the binding partner or the binding partner-macromolecule complex through a mucous membrane, e.g., through the intestinal mucosa, causes the immune system to tolerate the binding partner or the binding partner-macromolecule complex better than if the binding partner or the binding partner-macromolecule complex were, for example, injected. Thus, a lower titer of antibodies against the binding partner or the binding partner-macromolecule complex can be produced in the subject by delivering the binding partner or the binding partner-macromolecule complex with a delivery construct of the invention through the mucosa rather than injecting the binding partner or the binding partner-macromolecule complex, for example, subcutaneously, intravenously, intra-arterially, intraperitoneally, or otherwise. Generally, the time at which the lower titer of antibodies detected for the alternate routes of administration is detected should be roughly comparable; for example, the titer of antibodies can be determined at about 1 week, at about 2 weeks, at about 3 weeks, at about 4 weeks, at about 2 months, or at about 6 months following administration of the binding partner or the binding partner-macromolecule complex with the delivery construct or by injection.

Accordingly, in certain embodiments, the invention provides a method for delivering a binding partner to the bloodstream a subject that comprises contacting a delivery construct of the invention that comprises the binding partner to be delivered to an apical surface of a polarized epithelial cell of the subject, such that the binding partner is administered to the bloodstream of the subject, wherein a lower titer of antibodies specific for the binding partner is induced in the serum of the subject than is induced by subcutaneously administering the binding partner separately from the remainder of the delivery construct to a subject. In other embodiments, the invention provides a method for delivering a binding partner-macromolecule complex to the bloodstream a subject that comprises contacting a delivery construct of the invention that comprises the binding partner and the macromolecule to be delivered to an apical surface of a polarized epithelial cell of the subject, such that the binding partner-macromolecule complex is administered to the bloodstream of the subject, wherein a lower titer of antibodies specific for the binding partner-macromolecule complex is induced in the serum of the subject than is induced by subcutaneously administering the binding partner-macromolecule complex separately from the remainder of the delivery construct to a subject.

In certain embodiments, the titer of antibodies specific for the binding partner or the binding partner-macromolecule complex induced in the serum of the subject by the binding partner or the binding partner-macromolecule complex delivered by the delivery construct is less than about 95% of the titer of antibodies induced by subcutaneously administering the binding partner or the binding partner-macromolecule complex separately from the remainder of the delivery construct. In certain embodiments, the titer of antibodies specific for the binding partner or the binding partner-macromolecule complex induced in the serum of the subject by the binding partner or the binding partner-macromolecule complex delivered by the delivery construct is less than about 90% of the titer of antibodies induced by subcutaneously administering the binding partner or the binding partner-macromolecule complex separately from the remainder of the delivery construct. In certain embodiments, the titer of antibodies specific for the binding partner or the binding partner-macromolecule complex induced in the serum of the subject by the binding partner or the binding partner-macromolecule complex delivered by the delivery construct is less than about 85% of the titer of antibodies induced by subcutaneously administering the binding partner or the binding partner-macromolecule complex separately from the remainder of the delivery construct. In certain embodiments, the titer of antibodies specific for the binding partner or the binding partner-macromolecule complex induced in the serum of the subject by the binding partner or the binding partner-macromolecule complex delivered by the delivery construct is less than about 80% of the titer of antibodies induced by subcutaneously administering the binding partner or the binding partner-macromolecule complex separately from the remainder of the delivery construct. In certain embodiments, the titer of antibodies specific for the binding partner or the binding partner-macromolecule complex induced in the serum of the subject by the binding partner or the binding partner-macromolecule complex delivered by the delivery construct is less than about 75% of the titer of antibodies induced by subcutaneously administering the binding partner or the binding partner-macromolecule complex separately from the remainder of the delivery construct.

In certain embodiments, the titer of antibodies specific for the binding partner or the binding partner-macromolecule complex induced in the serum of the subject by the binding partner or the binding partner-macromolecule complex delivered by the delivery construct is less than about 70% of the titer of antibodies induced by subcutaneously administering the binding partner or the binding partner-macromolecule complex separately from the remainder of the delivery construct. In certain embodiments, the titer of antibodies specific for the binding partner or the binding partner-macromolecule complex induced in the serum of the subject by the binding partner or the binding partner-macromolecule complex delivered by the delivery construct is less than about 65% of the titer of antibodies induced by subcutaneously administering the binding partner or the binding partner-macromolecule complex separately from the remainder of the delivery construct. In certain embodiments, the titer of antibodies specific for the binding partner or the binding partner-macromolecule complex induced in the serum of the subject by the binding partner or the binding partner-macromolecule complex delivered by the delivery construct is less than about 60% of the titer of antibodies induced by subcutaneously administering the binding partner or the binding partner-macromolecule complex separately from the remainder of the delivery construct. In certain embodiments, the titer of antibodies specific for the binding partner or the binding partner-macromolecule complex induced in the serum of the subject by the binding partner or the binding partner-macromolecule complex delivered by the delivery construct is less than about 55% of the titer of antibodies induced by subcutaneously administering the binding partner or the binding partner-macromolecule complex separately from the remainder of the delivery construct. In certain embodiments, the titer of antibodies specific for the binding partner or the binding partner-macromolecule complex induced in the serum of the subject by the binding partner or the binding partner-macromolecule complex delivered by the delivery construct is less than about 55% of the titer of antibodies induced by subcutaneously administering the binding partner or the binding partner-macromolecule complex separately from the remainder of the delivery construct.

In certain embodiments, the titer of antibodies specific for the binding partner or the binding partner-macromolecule complex induced in the serum of the subject by the binding partner or the binding partner-macromolecule complex delivered by the delivery construct is less than about 50% of the titer of antibodies induced by subcutaneously administering the binding partner or the binding partner-macromolecule complex separately from the remainder of the delivery construct. In certain embodiments, the titer of antibodies specific for the binding partner or the binding partner-macromolecule complex induced in the serum of the subject by the binding partner or the binding partner-macromolecule complex delivered by the delivery construct is less than about 45% of the titer of antibodies induced by subcutaneously administering the binding partner or the binding partner-macromolecule complex separately from the remainder of the delivery construct. In certain embodiments, the titer of antibodies specific for the binding partner or the binding partner-macromolecule complex induced in the serum of the subject by the binding partner or the binding partner-macromolecule complex delivered by the delivery construct is less than about 40% of the titer of antibodies induced by subcutaneously administering the binding partner or the binding partner-macromolecule complex separately from the remainder of the delivery construct. In certain embodiments, the titer of antibodies specific for the binding partner or the binding partner-macromolecule complex induced in the serum of the subject by the binding partner or the binding partner-macromolecule complex delivered by the delivery construct is less than about 35% of the titer of antibodies induced by subcutaneously administering the binding partner or the binding partner-macromolecule complex separately from the remainder of the delivery construct. In certain embodiments, the titer of antibodies specific for the binding partner or the binding partner-macromolecule complex induced in the serum of the subject by the binding partner or the binding partner-macromolecule complex delivered by the delivery construct is less than about 30% of the titer of antibodies induced by subcutaneously administering the binding partner or the binding partner-macromolecule complex separately from the remainder of the delivery construct.

In certain embodiments, the titer of antibodies specific for the binding partner or the binding partner-macromolecule complex induced in the serum of the subject by the binding partner or the binding partner-macromolecule complex delivered by the delivery construct is less than about 25% of the titer of antibodies induced by subcutaneously administering the binding partner or the binding partner-macromolecule complex separately from the remainder of the delivery construct. In certain embodiments, the titer of antibodies specific for the binding partner or the binding partner-macromolecule complex induced in the serum of the subject by the binding partner or the binding partner-macromolecule complex delivered by the delivery construct is less than 20% of the titer of antibodies induced by subcutaneously administering the binding partner or the binding partner-macromolecule complex separately from the remainder of the delivery construct. In certain embodiments, the titer of antibodies specific for the binding partner or the binding partner-macromolecule complex induced in the serum of the subject by the binding partner or the binding partner-macromolecule complex delivered by the delivery construct is less than about 15% of the titer of antibodies induced by subcutaneously administering the binding partner or the binding partner-macromolecule complex separately from the remainder of the delivery construct. In certain embodiments, the titer of antibodies specific for the binding partner or the binding partner-macromolecule complex induced in the serum of the subject by the binding partner or the binding partner-macromolecule complex delivered by the delivery construct is less than about 10% of the titer of antibodies induced by subcutaneously administering the binding partner or the binding partner-macromolecule complex separately from the remainder of the delivery construct. In certain embodiments, the titer of antibodies specific for the binding partner or the binding partner-macromolecule complex induced in the serum of the subject by the binding partner or the binding partner-macromolecule complex delivered by the delivery construct is less than about 5% of the titer of antibodies induced by subcutaneously administering the binding partner or the binding partner-macromolecule complex separately from the remainder of the delivery construct. In certain embodiments, the titer of antibodies specific for the binding partner or the binding partner-macromolecule complex induced in the serum of the subject by the binding partner or the binding partner-macromolecule complex delivered by the delivery construct is less than about 1% of the titer of antibodies induced by subcutaneously administering the binding partner or the binding partner-macromolecule complex separately from the remainder of the delivery construct.

5.5.1. Methods of Administration

The delivery constructs of the invention can be administered to a subject by any method known to one of skill in the art. In certain embodiments, the delivery constructs are contacted to a mucosal membrane of the subject. For example, the mucosal membrane can be present in the eye, nose, mouth, trachea, lungs, esophagus, stomach, small intestine, large intestine, rectum, anus, sweat glands, vulva, vagina, or penis of the subject. Preferably, the mucosal membrane is a mucosal membrane present in the digestive tract of the subject, such as a mucosal membrane in the mouth, esophagus, stomach, small intestine, large intestine, or rectum of the subject.

In such embodiments, the delivery constructs are preferably administered to the subject orally. Thus, the delivery construct can be formulated to protect the delivery construct from degradation in the acid environment of the stomach, if necessary. For example, many embodiments of the delivery constructs of the invention comprise polypeptide domains with defined activities. Unless such delivery constructs are protected from acid and/or enzymatic hydrolysis in the stomach, the constructs will generally be digested before delivery of substantial amounts of the binding partner or the binding partner-macromolecule complex to be delivered. Accordingly, composition formulations that protect the delivery construct from degradation can be used in administration of these delivery constructs.

5.5.2. Dosage

Generally, a pharmaceutically effective amount of the delivery construct of the invention is administered to a subject. The skilled artisan can readily determine if the dosage of the delivery construct is sufficient to deliver an effective amount of the macromolecule, as described below. In certain embodiments, between about 1 μg and about 1 g of delivery construct is administered. In other embodiments, between about 10 μg and about 500 mg of delivery construct is administered. In still other embodiments, between about 10 μg and about 100 mg of delivery construct is administered. In yet other embodiments, between about 10 μg and about 1000 μg of delivery construct is administered. In still other embodiments, between about 10 μg and about 250 μg of delivery construct is administered. In yet other embodiments, between about 10 μg and about 100 μg of delivery construct is administered. Preferably, between about 10 μg and about 50 μg of delivery construct is administered.

The volume of a composition comprising the delivery construct that is administered will generally depend on the concentration of delivery construct and the formulation of the composition. In certain embodiments, a unit dose of the delivery construct composition is between about 0.05 ml and about 1 ml, preferably about 0.5 ml. The delivery construct compositions can be prepared in dosage forms containing between 1 and 50 doses (e.g., 0.5 ml to 25 ml), more usually between 1 and 10doses (e.g., 0.5 ml to 5 ml)

The delivery construct compositions of the invention can be administered in one dose or in multiple doses. A dose can be followed by one or more doses spaced by about 1 to about 6 hours, by about 6 to about 12 hours, by about 12 to about 24 hours, by about 1 day to about 3 days, by about 1 day to about 1 week, by about 1 week to about 2 weeks, by about 2 weeks to about 1 month, by about 4 to about 8 weeks, by about 1 to about 3 months, or by about 1 to about 6 months.

The binding partners to be delivered are generally binding partners for which a large amount of knowledge regarding dosage, frequency of administration, and methods for assessing effective concentrations in subjects has accumulated. Such knowledge can be used to assess efficiency of delivery, effective concentration of the binding partners in the subject, and frequency of administration. Thus, the knowledge of those skilled in the art can be used to determine whether, for example, the amount of binding partners delivered to the subject is an effective amount, the dosage should be increased or decreased, the subject should be administered the delivery construct more or less frequently, and the like.

5.5.3. Determining Amounts of Binding Partner/Binding Partner-Macromolecule Complexes Delivered

The methods of the invention can be used to deliver, either locally or systemically, a pharmaceutically effective amount of a binding partner or a binding partner-macromolecule complex to a subject. The skilled artisan can determine whether the methods result in delivery of such a pharmaceutically effective amount of the binding partner or the binding partner-macromolecule complex. The exact methods will depend on the binding partner or the binding partner-macromolecule complex that is delivered, but generally will rely on either determining the concentration of the binding partner or the binding partner-macromolecule complex in the blood of the subject or in the biological compartment of the subject where the binding partner or the binding partner-macromolecule exerts its effects. Alternatively or additionally, the effects of the binding partner or the binding partner-macromolecule on the subject can be monitored.

For example, in certain embodiments of the present invention, the binding partner that is delivered is insulin, e.g., human insulin. In such embodiments, the skilled artisan can determine whether a pharmaceutically effective amount of human insulin had been delivered to the subject by, for example, taking a plasma sample from the subject and determining the concentration of human insulin therein. One exemplary method for determining the concentration of human insulin is by performing an ELISA assay, but any other suitable assay known to the skilled artisan can be used.

Alternatively, one of skill in the art can determine if an effective amount of human insulin had been delivered to the subject by monitoring the blood sugar concentrations of the subject. As is well-known in the art, human insulin, among other activities, acts on hepatocytes to promote glycogen formation, thereby reducing plasma glucose concentrations. Accordingly, the subject's plasma glucose concentration can be monitored to determine whether an effective amount of insulin had been delivered.

Any effect of a binding partner or a binding partner-macromolecule complex that is administered that is known by one of skill in the art, without limitation, can be assessed in determining whether an effective amount of the binding partner or the binding partner-macromolecule complex has been administered. Exemplary effects include, but are not limited to, receptor binding, receptor activation, downstream effects of receptor binding, downstream effects of receptor activation, coordination of compounds, effective blood clotting, bone growth, wound healing, cellular proliferation, etc. The exact effect that is assessed will depend on the binding partner or the binding partner-macromolecule complex that is delivered.

5.6. Diagnostic Uses of Delivery Constructs

The delivery constructs of the invention can be used for diagnostic purposes to detect, diagnose, or monitor disorders. In a specific embodiment, diagnosis comprises: a) administering (for example, orally) to a subject an effective amount of a delivery construct of the invention comprising a labeled binding partner; b) waiting for a time interval following the administration for permitting the labeled binding partner to preferentially concentrate at sites in the subject where the antigen of interest is expressed (and for unbound labeled binding partner to be cleared to background level); c) determining background level; and d) detecting the labeled binding partner in the subject, such that detection of labeled binding partner above the background level indicates that the subject has the disorder. In accordance with this embodiment, the binding partner is labeled with an imaging moiety which is detectable using an imaging system known to one of skill in the art. Background level can be determined by various methods including, comparing the amount of labeled binding partner detected to a standard value previously determined for a particular system.

It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of^{99m}Tc. The labeled binding partner will then preferentially accumulate at the location of cells which contain the specific protein. In vivo tumor imaging is described in S. W. Burchiel et al., “Immunopharmacokinetics of Radiolabeled Antibodies and Their Fragments,” Chapter 13 in Tumor Imaging: The Radiochemical Detection of Cancer, S. W. Burchiel and B. A. Rhodes, eds., Masson Publishing Inc. (1982).

Depending on several variables, including the type of label used and the mode of administration, the time interval following the administration for permitting the labeled binding partner to preferentially concentrate at sites in the subject and for unbound labeled binding partner to be cleared to background level is 6 to 48 hours or 6 to 24 hours or 6 to 12 hours. In another embodiment the time interval following administration is 5 to 20 days or 5 to 10 days.

In one embodiment, monitoring of a disorder is carried out by repeating the method for diagnosing the disorder, for example, one month after initial diagnosis, six months after initial diagnosis, one year after initial diagnosis, etc.

Presence of the labeled binding partner can be detected in the subject using methods known in the art for in vivo scanning. These methods depend upon the type of label used. Skilled artisans will be able to determine the appropriate method for detecting a particular label. Non-limiting examples of labels include technetium (⁹⁹Tc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P, ¹⁵³Gd, ¹⁶⁹Yb, ⁵¹Cr, ⁵⁴Mn, ⁷⁵Se, ¹¹³Sn, and ¹¹⁷Tin. Methods and devices that may be used in the diagnostic methods of the invention include, but are not limited to, computed tomography (CT), whole body scan such as position emission tomography (PET), magnetic resonance imaging (MRI), and sonography.

In a specific embodiment, the binding partner is labeled with a radioisotope and is detected in the patient using a radiation responsive surgical instrument (Thurston et al., U.S. Pat. No. 5,441,050). In another embodiment, the binding partner is labeled with a fluorescent compound and is detected in the patient using a fluorescence responsive scanning instrument. In another embodiment, the binding partner is labeled with a positron emitting metal and is detected in the patient using positron emission-tomography. In yet another embodiment, the binding partner is labeled with a paramagnetic label and is detected in a patient using magnetic resonance imaging (MRI).

5.7. Compositions Comprising Delivery Constructs

The delivery constructs of the invention can be formulated as compositions. The compositions are generally formulated appropriately for the immediate use intended for the delivery construct. For example, if the delivery construct is not to be administered immediately, the delivery construct can be formulated in a composition suitable for storage. One such composition is a lyophilized preparation of the delivery construct together with a suitable stabilizer. Alternatively, the delivery construct composition can be formulated for storage in a solution with one or more suitable stabilizers. Any such stabilizer known to one of skill in the art without limitation can be used. For example, stabilizers suitable for lyophilized preparations include, but are not limited to, sugars, salts, surfactants, proteins, chaotropic agents, lipids, and amino acids. Stabilizers suitable for liquid preparations include, but are not limited to, sugars, salts, surfactants, proteins, chaotropic agents, lipids, and amino acids. Specific stabilizers than can be used in the compositions include, but are not limited to, trehalose, serum albumin, phosphatidylcholine, lecithin, and arginine. Other compounds, compositions, and methods for stabilizing a lyophilized or liquid preparation of the delivery constructs may be found, for example, in U.S. Pat. Nos. 6,573,237, 6,525,102, 6,391,296, 6,255,284, 6,133,229, 6,007,791, 5,997,856, and 5,917,021.

Further, the delivery construct compositions of the invention can be formulated for administration to a subject. Such vaccine compositions generally comprise one or more delivery constructs of the invention and a pharmaceutically acceptable excipient, diluent, carrier, or vehicle. Any such pharmaceutically acceptable excipient, diluent, carrier, or vehicle known to one of skill in the art without limitation can be used. Examples of a suitable excipient, diluent, carrier, or vehicle can be found in Remington's Pharmaceutical Sciences, 21st Ed. 2005, Mack Publishing Co., Easton.

In certain embodiments, the delivery construct compositions are formulated for oral administration. In such embodiments, the compositions are formulated to protect the delivery construct from acid and/or enzymatic degradation in the stomach. Upon passage to the neutral to alkaline environment of the duodenum, the delivery construct then contacts a mucous membrane and is transported across the polarized epithelial membrane. The delivery constructs may be formulated in such compositions by any method known by one of skill in the art, without limitation.

In certain embodiments, the oral formulation comprises a delivery construct and one or more compounds that can protect the delivery construct while it is in the stomach. For example, the protective compound should be able to prevent acid and/or enzymatic hydrolysis of the delivery construct. In certain embodiments, the oral formulation comprises a delivery construct and one or more compounds that can facilitate transit of the construct from the stomach to the small intestine. In certain embodiments, the one or more compounds that can protect the delivery construct from degradation in the stomach can also facilitate transit of the construct from the stomach to the small intestine. Preferably, the oral formulation comprises one or more compounds that can protect the delivery construct from degradation in the stomach and facilitate transit of the construct from the stomach to the small intestine. For example, inclusion of sodium bicarbonate can be useful in facilitating the rapid movement of intra-gastric delivered materials from the stomach to the duodenum as described in Mrsny et al., 1999, Vaccine 17:1425-1433.

Other methods for formulating compositions so that the delivery constructs can pass through the stomach and contact polarized epithelial membranes in the small intestine include, but are not limited to, enteric-coating technologies as described in DeYoung, 1989, Int J Pancreatol. 5 Suppl:31-6, and the methods provided in U.S. Pat. Nos. 6,613,332, 6,174,529, 6,086,918, 5,922,680, and 5,807,832.

The carrier constructs and binding partners of the invention can also be formulated as compositions. Appropriate formulations for these compositions include those described above for the delivery construct.

5.7.1. Kits Comprising Compositions

In yet another aspect, the invention provides a kit that comprises a composition of the invention. In certain embodiments, the kit further comprises instructions that direct administration of the composition to a mucous membrane of the subject to whom the composition is administered. In certain embodiments, the kit further comprises instructions that direct oral administration of the composition to the subject to whom the composition is administered.

In certain embodiments, the kit comprises a composition of the invention in more or more containers. In certain embodiments, the composition can be in a unit dosage form, e.g., a tablet, lozenge, capsule, etc. In certain embodiments, the composition can be provided in or with a device for administering the composition, such as, for example, a device configured to administer a single-unit dose of the composition, e.g., an inhaler.

5.8. Methods of Producing Delivery Constructs The delivery constructs of the invention may be produced by incubating a carrier construct (preferably, a purified carrier construct) and a binding partner (preferably, a purified binding partner) together under conditions permissible for non-covalent and/or covalent binding of the binding partner to the macromolecule of the carrier construct. In a specific embodiment, such conditions are those that are present physiologically when the binding partner and the macromolecule interact. Optionally, the delivery constructs formed by such an incubation may be separated from unbound carrier construct and/or unbound macromolecule using techniques known to one of skill in the art. For example, chromatography (e.g., affinity chromatography and ion chromatography), electrically-based methods (e.g., electrophoresis) and microwave can be used to separate the delivery construct from unbound carrier construct and/or unbound binding partner. Accordingly, in a specific embodiment, the delivery constructs are purified.

The delivery constructs of the invention may also be produced by co-expressing a carrier construct and a binding partner in cells engineered to comprise a first polynucleotide comprising a first nucleotide sequence encoding the carrier construct and a second polynucleotide comprising a second nucleotide sequence encoding the binding partner. Further, the delivery constructs of the invention may be produced by co-administering to a subject a first composition and a second composition, wherein the first composition comprising a carrier construct and the second composition comprises a binding partner.

In a preferred embodiment, the delivery constructs of the invention are not produced by happenstance in a subject. In other words, the invention does not encompass delivery constructs inadvertently produced in a subject as a result of a macromolecule of a carrier construct administered to the subject non-covalently binding to a binding partner present in the subject.

In accordance with the invention, the delivery constructs are formed prior to administration to a subject. Alternatively, the delivery constructs are formed following co-administration of a carrier construct and a binding partner. In accordance with this method, the carrier construct and the binding partner are administered simultaneously or within 1 minute, 2 minutes, 5 minutes, 10 minutes, 15 minutes, 30 minutes, 60 minutes, 2 hours 4 hours, 6 hours or within a day of each other with the intention of producing a delivery construct.

5.9. Recombinant Expression of Carrier Constructs

The carrier constructs of the invention are preferably produced recombinantly, as described below. However, the carrier constructs may also be produced by chemical synthesis using methods known to those of skill in the art.

5.9.1. Polynucleotides Encoding Carrier Constructs

In another aspect, the invention provides polynucleotides comprising a nucleotide sequence encoding the carrier constructs. These polynucleotides are useful, for example, for making the carrier constructs. In yet another aspect, the invention provides an expression system that comprises a recombinant polynucleotide sequence encoding a receptor-binding domain, a transcytosis domain, and a polylinker insertion site for a polynucleotide sequence encoding a macromolecule to which a binding partner binds. The polylinker insertion site can be anywhere in the polynucleotide sequence as long as the polylinker insertion does. not disrupt the receptor-binding domain or the transcytosis domain. In some embodiments, the polylinker insertion site is oriented near a polynucleotide sequence that encodes a cleavable linker so that cleavage at the cleavable linker separates a macromolecule encoded by a nucleic acid inserted into the polylinker insertion site from the remainder of the encoded carrier construct. Thus, in embodiments where the polylinker insertion site is at an end of the encoded construct, the polynucleotide comprises one nucleotide sequence encoding a cleavable linker between the polylinker insertion site and the remainder of the polynucleotide. In embodiments where the polylinker insertion site is not at the end of the encoded construct, the polylinker insertion site can be flanked by nucleotide sequences that each encode a cleavable linker.

In certain embodiments, the recombinant polynucleotides are based on polynucleotides encoding PE, or portions or derivatives thereof. In other embodiments, the recombinant polynucleotides are based on polynucleotides that hybridize to a polynucleotide that encodes PE under stringent hybridization conditions. A nucleotide sequence encoding PE is presented as SEQ ID NO.:3. This sequence can be used to prepare PCR primers for isolating a nucleic acid that encodes any portion of this sequence that is desired. For example, PCR can be used to isolate a nucleic acid that encodes one or more of the functional domains of PE. A nucleic acid so isolated can then be joined to nucleic acids encoding other functional domains of the carrier constructs using standard recombinant techniques.

Other in vitro methods that can be used to prepare a polynucleotide encoding PE, PE domains, or any other functional domain useful in the carrier constructs of the invention include, but are not limited to, reverse transcription, the polymerase chain reaction (PCR), the ligase chain reaction (LCR), the transcription-based amplification system (TAS), the self-sustained sequence replication system (3SR) and the QP replicase amplification system (QB). Any such technique known by one of skill in the art to be useful in construction of recombinant nucleic acids can be used. For example, a polynucleotide encoding the protein or a portion thereof can be isolated by polymerase chain reaction of cDNA using primers based on the DNA sequence of PE or a nucleotide encoding a receptor-binding domain.

Guidance for using these cloning and in vitro amplification methodologies are described in, for example, U.S. Pat. No. 4,683,195; Mullis et al., 1987, Cold Spring Harbor Symp. Quant. Biol. 51:263; and Erlich, ed., 1989, PCR Technology, Stockton Press, NY. Polynucleotides encoding a delivery construct or a portion thereof also can be isolated by screening genomic or cDNA libraries with probes selected from the sequences of the desired polynucleotide under stringent, moderately stringent, or highly stringent hybridization conditions.

Construction of nucleic acids encoding the carrier constructs of the invention can be facilitated by introducing an insertion site for a nucleic acid encoding the macromolecule into the construct. In certain embodiments, an insertion site for the antibody-binding domain can be introduced between the nucleotides encoding the cysteine residues of domain Ib. In other embodiments, the insertion site can be introduced anywhere in the nucleic acid encoding the construct so long as the insertion does not disrupt the functional domains encoded thereby. In certain embodiments, the insertion site can be in the ER retention domain.

In more specific embodiments, a nucleotide sequence encoding a portion of the Ib domain between the cysteine-encoding residues can be removed and replaced with a nucleotide sequence that includes a cloning site cleaved by a restriction enzyme. For example, the cloning site can be recognized and cleaved by PstI. In such examples, a polynucleotide encoding an antibody-binding domain that is flanked by PstI sequences can be inserted into the vector.

Further, the polynucleotides can also encode a secretory sequence at the amino terminus of the encoded carrier construct. Such constructs are useful for producing the carrier constructs in mammalian cells as they simplify isolation of the construct.

Furthermore, the polynucleotides of the invention also encompass derivative versions of polynucleotides encoding a carrier construct. Such derivatives can be made by any method known by one of skill in the art without limitation. For example, derivatives can be made by site-specific mutagenesis, including substitution, insertion, or deletion of one, two, three, five, ten or more nucleotides, of polynucleotides encoding the delivery construct. Alternatively, derivatives can be made by random mutagenesis. One method for randomly mutagenizing a nucleic acid comprises amplifying the nucleic acid in a PCR reaction in the presence of 0.1 mM MnCl₂ and unbalanced nucleotide concentrations. These conditions increase the misincorporation rate of the polymerase used in the PCR reaction and result in random mutagenesis of the amplified nucleic acid.

Several site-specific mutations and deletions in chimeric molecules derived from PE have been made and characterized. For example, deletion of nucleotides encoding amino acids 1-252 of PE yields a construct referred to as “PE40.” Deleting nucleotides encoding amino acids 1-279 of PE yields a construct referred to as “PE37.” See U.S. Pat. No. 5,602,095. In both of these constructs, the receptor-binding domain of PE, i.e., domain Ia, has been deleted. Nucleic acids encoding a receptor-binding domain can be ligated to these constructs to produce delivery constructs that are targeted to the cell surface receptor recognized by the receptor-binding domain. Of course, these recombinant polynucleotides are particularly useful for expressing delivery constructs that have a receptor-binding domain that is not domain Ia of PE. The recombinant polynucleotides can optionally encode an amino-terminal methionine to assist in expression of the construct. In certain embodiments, the receptor-binding domain can be ligated to the 5′ end of the polynucleotide encoding the transcytosis domain.

Other nucleic acids encoding mutant forms of PE that can be used as a source of nucleic acids for constructing the carrier constructs of the invention include, but are not limited to, PEΔ553 and those described in U.S. Pat. Nos. 5,602,095; 5,512,658 and 5,458,878, and in Vasil et al., 1986, Infect. Immunol. 52:538-48.

Accordingly, in certain embodiments, the invention provides a polynucleotide that encodes a carrier construct. The carrier construct comprises a receptor-binding domain, a transcytosis domain, a macromolecule to which a binding partner binds. Optionally, the carrier construct further comprises a cleavable linker. Cleavage at the cleavable linker can separate the macromolecule from the remainder of the construct. The cleavable linker can be cleaved by an enzyme that is present at a basal-lateral membrane of a polarized epithelial cell of the subject or in the plasma of the subject.

In certain embodiments, the polynucleotide hybridizes under stringent hybridization conditions to any polynucleotide of this invention. In further embodiments, the polynucleotide hybridizes under stringent conditions to a nucleic acid that encodes any carrier construct of the invention.

In certain embodiments, the polynucleotide encodes a carrier construct that further comprises a second cleavable linker. In certain embodiments, the first and/or second cleavable linker comprises an amino acid sequence that is selected from the group consisting of Ala-Ala-Pro-Phe (SEQ ID NO.:4), Gly-Gly-Phe (SEQ ID NO.:5), Ala-Ala-Pro-Val (SEQ ID NO.:6), Gly-Gly-Leu (SEQ ID NO.:7), Ala-Ala-Leu (SEQ ID NO.:8), Phe-Val-Arg (SEQ ID NO.:9), Val-Gly-Arg (SEQ ID NO.:10). In certain embodiments, the first and/or second cleavable linker encoded by the polynucleotide is cleavable by an enzyme that is selected from the group consisting of Cathepsin GI, Chymotrypsin I, Elastase I, Subtilisin AI, Subtilisin AII, Thrombin I, and Urokinase I.

In certain embodiments, the receptor-binding domain encoded by the polynucleotide is selected from the group consisting of receptor-binding domains from Pseudomonas exotoxin A, cholera toxin, diptheria toxin, shiga toxin, or shiga-like toxin; monoclonal antibodies; polyclonal antibodies; single-chain antibodies; TGF α; EGF; IGF-I; IGF-II; IGF-III; IL-1; IL-2; IL-3; IL-6; MIP-1a; MIP-1b; MCAF; and IL-8. In certain embodiments, the receptor-binding domain encoded by the polynucleotide binds to a cell-surface receptor that is selected from the group consisting of α2-macroglobulin receptor, EGFR, IGFR, transferrin receptor, chemokine receptor, CD25, CD11B, CD11C, CD80, CD86, TNFα receptor, TOLL receptor, M-CSF receptor, GM-CSF receptor, scavenger receptor, and VEGF receptor. In further embodiments, the receptor-binding domain encoded by the polynucleotide is Domain Ia of Pseudomonas exotoxin A. In a specific embodiment, the receptor-binding domain encoded by the polynucleotide has an amino acid sequence that is SEQ ID NO.:1.

In certain embodiments, the transcytosis domain encoded by the polynucleotide is selected from the group consisting of transcytosis domains from Pseudomonas exotoxin A, diptheria toxin, pertussis toxin, cholera toxin, heat-labile E. coli enterotoxin, shiga toxin, and shiga-like toxin. In further embodiments, the transcytosis domain is Pseudomonas exotoxin A transcytosis domain. In still further embodiments, the Pseudomonas exotoxin A transcytosis domain has an amino acid sequence that is SEQ ID NO.:2.

In other embodiments, the invention provides a polynucleotide that encodes a carrier construct that comprises a nucleic acid sequence encoding a receptor-binding domain, a nucleic acid sequence encoding a transcytosis domain, a nucleic acid sequence comprising a polylinker insertion site, and optionally a nucleic acid sequence encoding a cleavable linker. The polylinker insertion site can be oriented relative to the nucleic acid sequence encoding a cleavable linker to allow to cleavage of the cleavable linker to separate a macromolecule that is encoded by a nucleic acid inserted into the polylinker insertion site from the remainder of said delivery construct. The cleavable linker can be cleavable by an enzyme that is present at a basal-lateral membrane of a polarized epithelial cell of said subject or in the plasma of said subject.

5.9.2. Expression Vectors for Expressing Carrier Constructs

In still another aspect, the invention provides expression vectors for expressing the carrier constructs. Generally, expression vectors are recombinant polynucleotide molecules comprising expression control sequences operatively linked to a nucleotide sequence encoding a polypeptide. Expression vectors can readily be adapted for function in prokaryotes or eukaryotes by inclusion of appropriate promoters, replication sequences, selectable markers, etc. to result in stable transcription and translation of mRNA. Techniques for construction of expression vectors and expression of genes in cells comprising the expression vectors are well known in the art. See, e.g., Sambrook et al., 2001, Molecular Cloning—A Laboratory Manual, 3^{rd} edition, Cold Spring Harbor Laboratory, Cold Spring Harbor, N.Y., and Ausubel et al., eds., Current Edition, Current Protocols in Molecular Biology, Greene Publishing Associates and Wiley Interscience, NY.

Useful promoters for use in expression vectors include, but are not limited to, a metallothionein promoter, a constitutive adenovirus major late promoter, a dexamethasone-inducible MMTV promoter, a SV40 promoter, a MRP pol III promoter, a constitutive MPSV promoter, a tetracycline-inducible CMV promoter (such as the human immediate-early CMV promoter), and a constitutive CMV promoter. See Section 5.8 and 5.9, infra, for examples of other types of promoters.

The expression vectors should contain expression and replication signals compatible with the cell in which the carrier constructs are expressed. Expression vectors useful for expressing carrier constructs include viral vectors such as retroviruses, adenoviruses and adenoassociated viruses, plasmid vectors, cosmids, and the like. Viral and plasmid vectors are preferred for transfecting the expression vectors into mammalian cells. For example, the expression vector pcDNA1 (Invitrogen, San Diego, Calif.), in which the expression control sequence comprises the CMV promoter, provides good rates of transfection and expression into such cells. See Sections 5.8 and 5.9, infra, for examples of other types of expression vectors.

The expression vectors can be introduced into the cell for expression of the carrier constructs by any method known to one of skill in the art without limitation. Such methods include, but are not limited to, e.g., direct uptake of the molecule by a cell from solution; facilitated uptake through lipofection using, e.g., liposomes or immunoliposomes; particle-mediated transfection; etc. See, e.g., U.S. Pat. No. 5,272,065; Goeddel et al., eds, 1990, Methods in Enzymology, vol. 185, Academic Press, Inc., CA; Krieger, 1990, Gene Transfer and Expression—A Laboratory Manual, Stockton Press, NY; Sambrook et al., 1989, Molecular Cloning—A Laboratory Manual, Cold Spring Harbor Laboratory, NY; and Ausubel et al., eds., Current Edition, Current Protocols in Molecular Biology, Greene Publishing Associates and Wiley Interscience, NY. See Sections 5.8 and 5.9, infra, for examples of other methods of introducing expression vectors into cells and for methods of producing stable cells containing expression vectors.

The expression vectors can also contain a purification moiety that simplifies isolation of the carrier construct. For example, a polyhistidine moiety of, e.g., six histidine residues, can be incorporated at the amino terminal end of the protein. The polyhistidine moiety allows convenient isolation of the protein in a single step by nickel-chelate chromatography. In certain embodiments, the purification moiety can be cleaved from the remainder of the carrier construct following purification. In other embodiments, the moiety does not interfere with the function of the functional domains of the carrier construct and thus need not be cleaved.

5.9.3. Cell for Expressing a Carrier Construct

In yet another aspect, the invention provides a cell comprising an expression vector for expression of the carrier constructs, or portions thereof. The cell is preferably selected for its ability to express high concentrations of the carrier construct to facilitate purification of the protein. In certain embodiments, the cell is a prokaryotic cell, for example, E. coli. As described in the examples, the carrier constructs are properly folded and comprise the appropriate disulfide linkages when expressed in E. coli.

In other embodiments, the cell is a eukaryotic cell. Useful eukaryotic cells include yeast and mammalian cells. Any mammalian cell known by one of skill in the art to be useful for expressing a recombinant polypeptide, without limitation, can be used to express the delivery constructs. For example, Chinese hamster ovary (CHO) cells can be used to express the carrier constructs. See, e.g., Sections 5.8 and 5.9, infra, for additional examples of cell types that may be used to express a carrier construct.

5.10. Recombinant Expression of Binding Partners

Binding partners can be produced by standard recombinant DNA techniques or by protein synthetic techniques, e.g., by use of a peptide synthesizer. For example, a nucleic acid molecule encoding a binding partner can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, e.g., Current Protocols in Molecular Biology, Ausubel et al., eds., John Wiley & Sons, 1992).

The nucleotide sequence encoding a binding partner may be obtained from any information available to those of skill in the art (e.g., from Genbank, the literature, or by routine cloning). The nucleotide sequence coding for a binding partner can be inserted into an appropriate expression vector, i.e., a vector which contains the necessary elements for the transcription and translation of the inserted protein-coding sequence. A variety of host-vector systems may be utilized in the present invention to express the protein-coding sequence. These include but are not limited to mammalian cell systems infected with virus (e.g., vaccinia virus, adenovirus, etc.); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used.

The expression of a binding partner may be controlled by any promoter or enhancer element known in the art. Promoters which may be used to control the expression of the gene encoding binding partner include, but are not limited to, the SV40 early promoter region (Bernoist and Chambon, Nature, 290:304-310, 1981), the promoter contained in the 3′ long terminal repeat of Rous sarcoma virus (Yamamoto, et al. , Cell, 22:787-797, 1980), the herpes thymidine kinase promoter (Wagner et al., Proc. Natl. Acad. Sci. U.S.A., 78:1441-1445, 1981), the regulatory sequences of the metallothionein gene (Brinster et al., Nature, 296:39-42, 1982), the tetracycline (Tet) promoter (Gossen et al., Proc. Nat. Acad. Sci. USA, 89:5547-5551, 1995); prokaryotic expression vectors such as the β-lactamase promoter (Villa-Kamaroff, et al., Proc. Natl. Acad. Sci. U.S.A., 75:3727-3731, 1978), or the tac promoter (DeBoer, et al., Proc. Natl. Acad Sci. U.S.A., 80:21-25, 1983; see also “Useful proteins from recombinant bacteria” in Scientific American, 242:74-94, 1980); plant expression vectors comprising the nopaline synthetase promoter region (Herrera-Estrella et al., Nature, 303:209-213, 1983) or the cauliflower mosaic virus 35S RNA promoter (Gardner, et al., Nucl. Acids Res., 9:2871, 1981), and the promoter of the photosynthetic enzyme ribulose biphosphate carboxylase (Herrera-Estrella et al., Nature, 310:115-120, 1984); promoter elements from yeast or other fungi such as the Gal 4 promoter, the ADC (alcohol dehydrogenase) promoter, PGK (phosphoglycerol kinase) promoter, alkaline phosphatase promoter, and the following animal transcriptional control regions, which exhibit tissue specificity and have been utilized in transgenic animals: elastase I gene control region which is active in pancreatic acinar cells (Swift et al., Cell 38:639-646, 1984; Omitz et al., 50:399-409, Cold Spring Harbor Symp. Quant. Biol., 1986; MacDonald, Hepatology 7:425-515, 1987); insulin gene control region which is active in pancreatic beta cells (Hanahan, Nature 315:115-122, 1985), immunoglobulin gene control region which is active in lymphoid cells (Grosschedl et al., Cell, 38:647-658, 1984; Adames et al., Nature 318:533-538, 1985; Alexander et al., Mol. Cell. Biol., 7:1436-1444, 1987), mouse mammary tumor virus control region which is active in testicular, breast, lymphoid and mast cells (Leder et al., Cell, 45:485-495, 1986), albumin gene control region which is active in liver (Pinkert et al. , Genes and Devel., 1:268-276, 1987), alpha-fetoprotein gene control region which is active in liver (Krumlauf et al., Mol. Cell. Biol., 5:1639-1648, 1985; Hammer et al., Science, 235:53-58, 1987; alpha1-antitrypsin gene control region which is active in the liver (Kelsey et al., Genes and Devel., 1:161-171, 1987), beta-globin gene control region which is active in myeloid cells (Mogram et al., Nature, 315:338-340, 1985; Kollias et al., Cell, 46:89-94, 1986; myelin basic protein gene control region which is active in oligodendrocyte cells in the brain (Readhead et al., Cell, 48:703-712, 1987); myosin light chain-2 gene control region which is active in skeletal muscle (Sani, Nature, 314:283-286, 1985); neuronal-specific enolase (NSE) which is active in neuronal cells (Morelli et al., Gen. Virol., 80:571-83, 1999); brain-derived neurotrophic factor (BDNF) gene control region which is active in neuronal cells (Tabuchi et al., Biochem. Biophysic. Res. Comprising, 253:818-823, 1998); glial fibrillary acidic protein (GFAP) promoter which is active in astrocytes (Gomes et al., Braz. J. Med. Biol. Res., 32(5):619-631, 1999; Morelli et al., Gen. Virol., 80:571-83, 1999) and gonadotropic releasing hormone gene control region which is active in the hypothalamus (Mason et al., Science, 234:1372-1378, 1986).

In a specific embodiment, the expression of a binding partner is regulated by a constitutive promoter. In another embodiment, the expression of a binding partner is regulated by an inducible promoter. In accordance with these embodiments, the promoter may be a tissue-specific promoter.

In a specific embodiment, a vector is used that comprises a promoter operably linked to a binding partner -encoding nucleic acid, one or more origins of replication, and, optionally, one or more selectable markers (e.g., an antibiotic resistance gene).

In mammalian host cells, a number of viral-based expression systems may be utilized. In cases where an adenovirus is used as an expression vector, the binding partner coding sequence may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts (e.g., see Logan & Shenk, Proc. Natl. Acad. Sci. USA, 81:355-359, 1984). Specific initiation signals may also be required for efficient translation of inserted binding partner coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bitter et al., Methods in Enzymol. 153:516-544, 1987).

Expression vectors containing inserts of a gene encoding a binding partner can be identified by three general approaches: (a) nucleic acid hybridization, (b) presence or absence of “marker” gene functions, and (c) expression of inserted sequences. In the first approach, the presence of a gene encoding a binding partner in an expression vector can be detected by nucleic acid hybridization using probes comprising sequences that are homologous to an inserted gene encoding the binding partner. In the second approach, the recombinant vector/host system can be identified and selected based upon the presence or absence of certain “marker” gene functions (e.g., thymidine kinase activity, resistance to antibiotics, transformation phenotype, occlusion body formation in baculovirus, etc.) caused by the insertion of a nucleotide sequence encoding a binding partner in the vector. For example, if the nucleotide sequence encoding the binding partner is inserted within the marker gene sequence of the vector, recombinants containing the gene encoding the binding partner insert can be identified by the absence of the marker gene function. In the third approach, recombinant expression vectors can be identified by assaying the gene product (i.e., binding partner) expressed by the recombinant. Such assays can be based, for example, on the physical or functional properties of the binding partner in in vitro assay systems, e.g., binding with anti-binding partner antibody.

In addition, a host cell strain may be chosen which modulates the expression of the inserted sequences, or modifies and processes the gene product in the specific fashion desired. Expression from certain promoters can be elevated in the presence of certain inducers; thus, expression of the genetically engineered binding partner may be controlled. Furthermore, different host cells have characteristic and specific mechanisms for the translational and post-translational processing and modification (e.g., glycosylation, phosphorylation of proteins). Appropriate cell lines or host systems can be chosen to ensure the desired modification and processing of the foreign protein expressed. For example, expression in a bacterial system will produce an unglycosylated product and expression in yeast will produce a glycosylated product. Eukaryotic host cells which possess the cellular machinery for proper processing of the primary transcript, glycosylation, and phosphorylation of the gene product may be used. Such mammalian host cells include but are not limited to CHO, VERY, BHK, HeLa, COS, MDCK, 293, 3T3, WI38, and in particular, neuronal cell lines such as, for example, SK-N-AS, SK-N-FI, SK-N-DZ human neuroblastomas (Sugimoto et al., J. Natl. Cancer Inst., 73: 51-57, 1984), SK-N-SH human neuroblastoma (Biochim. Biophys. Acta, 704: 450-460, 1982), Daoy human cerebellar medulloblastoma (He et al., Cancer Res., 52: 1144-1148, 1992) DBTRG-05MG glioblastoma cells (Kruse et al., 1992, In Vitro Cell. Dev. Biol., 28A:609-614, 1992), IMR-32 human neuroblastoma (Cancer Res., 30: 2110-2118, 1970), 1321N1 human astrocytoma (Proc. Natl Acad. Sci. USA, 74: 4816, 1997), MOG-G-CCM human astrocytoma (Br. J. Cancer, 49: 269, 1984), U87MG human glioblastoma-astrocytoma (Acta Pathol. Microbiol. Scand., 74: 465-486, 1968), A172 human glioblastoma (Olopade et al, Cancer Res., 52: 2523-2529, 1992), C6 rat glioma cells (Benda et al., Science, 161: 370-371, 1968), Neuro-2a mouse neuroblastoma (Proc. Natl. Acad. Sci. USA, 65: 129-136, 1970), NB41A3 mouse neuroblastoma (Proc. Natl. Acad. Sci. USA, 48: 1184-1190, 1962), SCP sheep choroid plexus (Bolin et al., J Virol. Methods, 48: 211-221, 1994), G355-5, PG-4 Cat normal astrocyte (Haapala et al., J. Virol., 53: 827-833, 1985), Mpf ferret brain (Trowbridge et al., In Vitro, 18: 952-960, 1982), and normal cell lines such as, for example, CTX TNA2 rat normal cortex brain (Radany et al., Proc. Natl. Acad. Sci. USA, 89: 6467-6471, 1992) such as, for example, CRL7030 and Hs578Bst. Furthermore, different vector/host expression systems may effect processing reactions to different degrees.

For long-term, high-yield production of recombinant proteins, stable expression is preferred. For example, cell lines which stably express the binding partner may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched medium, and then are switched to a selective medium. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines that express the differentially expressed or pathway gene protein. Such engineered cell lines may be particularly useful in screening and evaluation of compounds that affect the endogenous activity of the differentially expressed or pathway gene protein.

A number of selection systems may be used, including but not limited to the herpes simplex virus thymidine kinase (Wigler, et al., Cell, 11:223, 1997), hypoxanthine-guanine phosphoribosyltransferase (Szybalska & Szybalski, Proc. Natl. Acad. Sci. USA, 48:2026, 1962), and adenine phosphoribosyltransferase (Lowy, et al., 1980, Cell, 22:817, 1980) genes can be employed in tk-, hgprt- or aprt-cells, respectively. Also, antimetabolite resistance can be used as the basis of selection for dhfr, which confers resistance to methotrexate (Wigler, et al., Natl. Acad Sci. USA, 77:3567, 1980; O'Hare, et al., Proc. Natl. Acad. Sci. USA, 78:1527, 1981); gpt, which confers resistance to mycophenolic acid (Mulligan & Berg, Proc. Natl. Acad. Sci. USA, 78:2072, 1981); neo, which confers resistance to the aminoglycoside G-418 (Colberre-Garapin, et al., J. Mol. Biol., 150:1, 1981); and hygro, which confers resistance to hygromycin (Santerre, et al., Gene, 30:147, 1984) genes.

Once a binding partner of the invention has been produced by recombinant expression, it may be purified by any method known in the art for purification of a protein, for example, by chromatography (e.g., ion exchange, affinity, particularly by affinity for the specific antigen after Protein A, and sizing column chromatography), centrifugation, differential solubility, or by any other standard technique for the purification of proteins.

5.11. Biological Activity of Delivery Constructs

Having selected the domains of the carrier construct, the function of these domains, and of the delivery constructs as a whole, can be routinely tested to ensure that the constructs can deliver a binding partner and/or binding partner-macromolecule complex across mucous membranes of a subject free from the remainder of the construct. For example, the carrier constructs and/or delivery constructs can be tested for cell recognition, transcytosis and cleavage using routine assays. The entire carrier construct can be tested, or, the function of various domains can be tested by substituting them for native domains of the wild-type toxin.

5.11.1.1. Receptor-binding/Cell recognition

Receptor-binding domain function can be tested by monitoring the delivery construct's or carrier construct's ability to bind to the target receptor. Such testing can be accomplished using cell-based assays, with the target receptor present on a cell surface, or in cell-free assays. For example, delivery construct or carrier construct binding to a target can be assessed with affinity chromatography. The construct can be attached to a matrix in an affinity column, and binding of the receptor to the matrix detected, or vice versa. Alternatively, if antibodies have been identified that bind to either the receptor-binding domain or its cognate receptor, the antibodies can be used, for example, to detect the receptor-binding domain in the delivery construct or carrier construct by immunoassay, or in a competition assay for the cognate receptor. An exemplary cell-based assay that detects delivery construct or carrier construct binding to receptors on cells comprises labeling the construct and detecting its binding to cells by, e.g., fluorescent cell sorting, autoradiography, etc.

5.11.1.2. Transcytosis

The function of the transcytosis domain can be tested as a function of the delivery construct's or carrier construct's ability to pass through an epithelial membrane. Because transcytosis first requires binding to the cell, these assays can also be used to assess the function of the cell recognition domain.

The delivery construct's or carrier construct's transcytosis activity can be tested by any method known by one of skill in the art, without limitation. In certain embodiments, transcytosis activity can be tested by assessing the ability of a delivery construct or carrier construct to enter a non-polarized cell to which it binds. Without intending to be bound to any particular theory or mechanism of action, it is believed that the same property that allows a transcytosis domain to pass through a polarized epithelial cell also allows molecules bearing the transcytosis domain to enter non-polarized cells. Thus, the delivery construct's or carrier construct's ability to enter the cell can be assessed, for example, by detecting the physical presence of the construct in the interior of the cell. For example, the delivery construct or the carrier construct can be labeled with, for example, a fluorescent marker, and the delivery construct or carrier construct exposed to the cell. Then, the cells can be washed, removing any delivery construct or carrier construct that has not entered the cell, and the amount of label remaining determined. Detecting the label in this fraction indicates that the delivery construct or the carrier construct has entered the cell.

In other embodiments, the delivery construct's or carrier construct's transcytosis ability can be tested by assessing the delivery construct's or carrier construct's ability to pass through a polarized epithelial cell. For example, the delivery construct or carrier construct can be labeled with, for example, a fluorescent marker and contacted to the apical membranes of a layer of epithelial cells. Fluorescence detected on the basal-lateral side of the membrane formed by the epithelial cells indicates that the transcytosis domain is functioning properly.

5.11.1.3. Cleavable Linker Cleavage

The function of the cleavable linker can generally be tested in a cleavage assay. Any suitable cleavage assay known by one of skill in the art, without limitation, can be used to test the cleavable linkers. Both cell-based and cell-free assays can be used to test the ability of an enzyme to cleave the cleavable linkers.

An exemplary cell-free assay for testing cleavage of cleavable linkers comprises preparing extracts of polarized epithelial cells and exposing a labeled delivery construct or a labeled carrier construct bearing a cleavable linker to the fraction of the extract that corresponds to membrane-associated enzymes. In such assays, the label can be attached to either the macromolecule or to the remainder of the carrier construct. Among these enzymes are cleavage enzymes found near the basal-lateral membrane of a polarized epithelial cell, as described above. Cleavage can be detected, for example, by binding the carrier construct with, for example, an antibody and washing off unbound molecules. If label is attached to the macromolecule, then little or no label should be observed on the molecule bound to the antibodies. Alternatively, the binding agent used in the assay can be specific for the macromolecule, and the remainder of the construct can be labeled. In either case, cleavage can be assessed.

Cleavage can also be tested using cell-based assays that test cleavage by polarized epithelial cells assembled into membranes. For example, a labeled carrier construct, or portion of a carrier construct comprising the cleavable linker, can be contacted to either the apical or basolateral side of a monolayer of suitable epithelial cells, such as, for example, Coco-2 cells, under conditions that permit cleavage of the linker. Cleavage can be detected by detecting the presence or absence of the label using a reagent that specifically binds the carrier construct, or portion thereof. For example, an antibody specific for the carrier construct can be used to bind a carrier construct comprising a label distal to the cleavable linker in relation to the portion of the carrier construct bound by the antibody. Cleavage can then be assessed by detecting the presence of the label on molecules bound to the antibody. If cleavage has occurred, little or no label should be observed on the molecules bound to the antibody. By performing such experiments, enzymes that preferentially cleave at the basolateral membrane rather than the apical membrane can be identified, and, further, the ability of such enzymes to cleave the cleavable linker in a carrier construct can be confirmed.

Further, cleavage can also be tested using a fluorescence reporter assay as described in U.S. Pat. No. 6,759,207. Briefly, in such assays, the fluorescence reporter is contacted to the basolateral side of a monolayer of suitable epithelial cells under conditions that allow the cleaving enzyme to cleave the reporter. Cleavage of the reporter changes the structure of the fluorescence reporter, changing it from a non-fluorescent configuration to a fluorescent configuration. The amount of fluorescence observed indicates the activity of the cleaving enzyme present at the basolateral membrane.

Further, cleavage can also be tested using an intra-molecularly quenched molecular probe, such as those described in U.S. Pat. No. 6,592,847. Such probes generally comprise a fluorescent moiety that emits photons when excited with light of appropriate wavelength and a quencher moiety that absorbs such photons when in close proximity to the fluorescent moiety. Cleavage of the probe separates the quenching moiety from the fluorescent moiety, such that fluorescence can be detected, thereby indicating that cleavage has occurred. Thus, such probes can be used to identify and assess cleavage by particular cleaving enzymes by contacting the basolateral side of a monolayer of suitable epithelial cells with the probe under conditions that allow the cleaving enzyme to cleave the probe. The amount of fluorescence observed indicates the activity of the cleaving enzyme being tested.

5.11.2. Proper Folding of the Carrier Construct

To determine that a carrier construct has properly folded and is able to bind to a binding partner, an immunoassay can be performed. For example, an ELISA can be performed. Such an ELISA may comprise: coating a 96 well plate with a binding partner of interest, adding the carrier construct to the well and incubating for a period of time, and detecting the binding of the binding partner to the carrier construct. To detect the binding, a second detectably labeled antibody that recognizes the carrier construct can be added to the well.

5.11.3. Binding Affinity of Macromolecule

The binding affinity of a macromolecule of a carrier construct for a binding partner can be determined by competitive binding assays. One example of a competitive binding assay is a radioimmunoassay that involves incubation of labeled binding partner (e.g., ³H or ¹²⁵¹I) with the carrier construct of interest in the presence of increasing amounts of unlabeled binding partner, and the detection of the carrier construct bound to the labeled binding partner. The affinity of the macromolecule of the carrier construct for the binding partner and the binding off-rates can be determined from the saturation data by scatchard analysis. Competition with a second binding partner can also be determined using radioimmunoassays.

In a preferred embodiment, BIAcore kinetic analysis is used to determine the binding on and off rates of binding partner to a carrier construct. BIAcore kinetic analysis comprises analyzing the binding and dissociation of a carrier construct from chips with immobilized binding partners on their surface.

5.11.4. Activity of Delivery Construct

The delivery constructs and compositions of the invention are preferably tested in vitro, and then in vivo for the desired therapeutic or prophylactic activity, prior to use in humans. For example, in vitro assays which can be used to determine whether administration of a specific delivery construct or a composition of the present invention is indicated, include in vitro cell culture assays in which a subject tissue sample is grown in culture, and exposed to or otherwise administered the delivery construct or composition of the present invention, and the effect of such delivery construct or composition of the present invention upon the tissue sample is observed. In various specific embodiments, in vitro assays can be carried out with representative cells of cell types involved in a disorder, to determine if a delivery construct or composition of the present invention has a desired effect upon such cell types.

Delivery constructs or compositions of the present invention for use in preventing, treating, managing or ameliorating a disorder or a symptom thereof can be tested for their toxicity in suitable animal model systems, including but not limited to rats, mice, cows, monkeys, and rabbits. For in vivo testing for the toxicity of a delivery construct or a composition, any animal model system known in the art may be used.

5.11.5. Pharmacokinetic Assays

To assess the pharmacokinetics of an exemplary binding partner or binding partner-macromolecule complex delivered with a delivery construct, ELISA assays can used to measure serum concentrations of the binding partner or the binding partner-macromolecule complex at defined timepoints following administration. Serum concentration data obtained is used to compare the pharmacokinetics of the binding partner or the binding partner-macromolecule complex administered with the delivery construct to those observed with conventional methods administration (e.g., subcutaneous injection).

### 6. EXAMPLES

The following examples merely illustrate the invention, and are not intended to limit the invention in any way.

6.1. HGH-HGHBP Delivery Construct

6.1.1. HGH Carrier Construct

6.1.1.1. Construction of HGH Carrier Construct

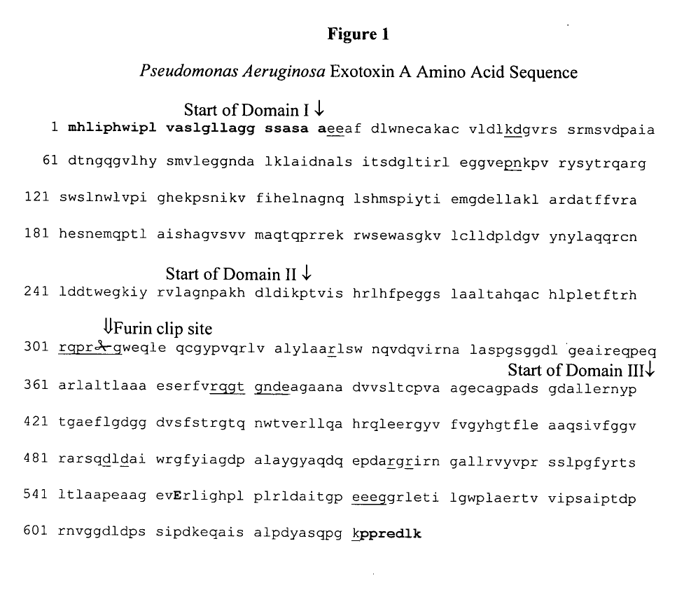

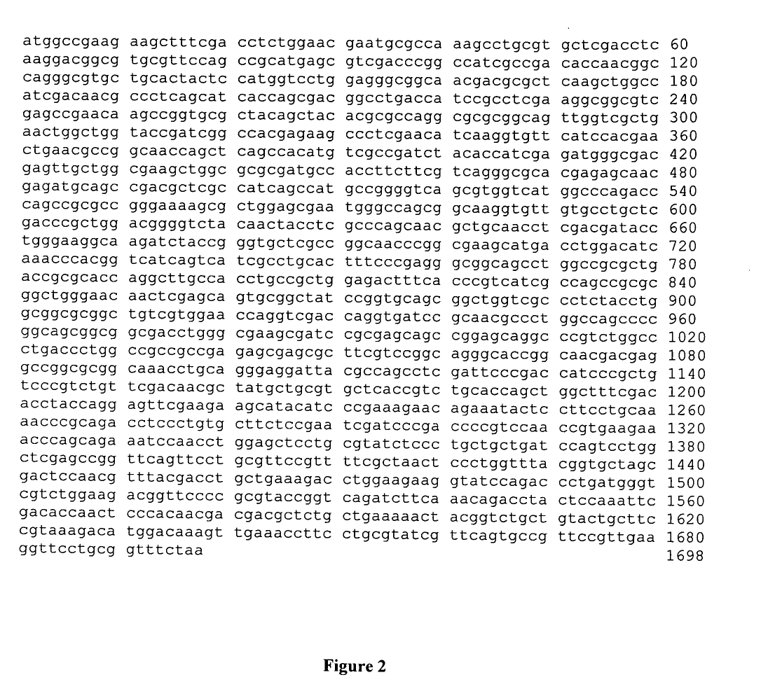

This example describes construction of an exemplary carrier construct comprising human growth hormone (hGH), termed Carrier Construct 1. The construct comprises sequences encoding Domains I and II of ntPE (amino acids 26-372 as shown in FIG. 1) and hGH (Accession No. P01244; see Seeburg et al., 1977, Nature 270:486-494 and Page et al., 1981, Nucleic Acids Res. 9:2087-2104), and are also tagged with a 6-His motif at the N-terminus of the polypeptide to facilitate purification. The final plasmid was verified by restriction enzyme digestions and DNA sequencing. The nucleotide sequence of the portion of the plasmid that encodes the exemplary carrier construct is presented as FIG. 2, while the amino acid sequence of the carrier construct is presented as FIG. 3.

6.1.1.2. Expression of HGH Carrier Construct

E. coli BL21(DE3) pLysS competent cells (Novagen, Madison, Wis.) were transformed using a standard heat-shock method in the presence of the appropriate plasmid to generate ntPE-human Growth Hormone (hGH) expression cells, selected on ampicillin-containing media, and isolated and grown in Luria-Bertani broth (Difco; Becton Dickinson, Franklin Lakes, N.J.) with antibiotic, then induced for protein expression by the addition of 1 mM isopropyl-D-thiogalactopyranoside (IPTG) at OD 0.6. Two hours following IPTG induction, cells were harvested by centrifugation at 5,000 rpm for 10 min. Inclusion bodies were isolated following cell lysis and proteins were solubilized in the buffer containing 100 mM Tris-HCl (pH 8.0), 2 mM EDTA, 6 M guanidine HCl, and 65 mM dithiothreitol. Solubilized His ntPE-rGH was refolded in the presence of 0.1 M Tris, pH=7.4, 500 mM L-arginine, 0.9 mM GSSG, and 2 mM EDTA. The refolded proteins were purified by Q sepharose Ion Exchange and Superdex 200 Gel Filtration chromatography (Amersham BioSciences, Inc., Sweden). The purity of proteins was assessed by SDS-PAGE and analytical HPLC (Agilent, Inc. Palo Alto, Calif.).

6.1.1.3. Characterization of HGH Carrier Construct

The following procedures can be used to assess proper refolding of a carrier construct. The protein refolding process is monitored by measuring, e.g., Carrier Construct 1 binding activity with the ntPE binding receptor, CD91 receptor, and with the cognate ligand for hGH, recombinant hGH binding protein (hGHBP) on a Biacore SPR instrument (Biacore, Sweden) according to the manufacturer's instructions. Proper refolding of other macromolecules in exemplary constructs can be tested in similar binding assays with appropriate binding agents. By testing such binding affinities, the skilled artisan can assess the proper folding of each portion of the carrier construct.

6.1.1.4. HGH Carrier Construct Cleavage Assays

This example describes experiments performed to identify and verify enzymes that can be used to cleave the cleavable linkers of the carrier constructs described herein. First, Caco-2 (ATCC Accession No. HTB-37) cells in passage 21 were obtained from American Type Culture Collection (Manassas, Va.). Human tracheal epithelial (HTE) cells were obtained from J. Whiddecombe of the Department of Physiology at the University of California, Davis Medical School. Caco-2 cells are routinely grown on 75 cm² plastic culture flasks (Becton Dickinson, Franklin Lakes, N.J.) in DMEM containing 10% fetal bovine serum and 1% penicillin-streptomycin at 37° C. in a 5% CO₂/95% air atmosphere. HTE cells are grown as described in Yamaya et al, 1992, Am J Physiol. 262(6 Pt 1):L713-24.

To identify suitable cleavable linkers, HTE or Caco-2 cells are seeded at a density of 5×10⁴ cells/cm² onto 24-well collagen-coated polycarbonate transwell filters (Corning, Acton, Mass.) for 12-14 days. Confluent monolayers achieve a transepithelial resistance (TER) of >500 ohm cm², as measured using an EVOM epithelial voltohmmeter and STX2 electrode (World Precision Instruments, Sarasota, Fla.). To determine specific enzyme activity, substrates specific for the tested peptidase (500 μM or 1 mM substrate in 250 μl DMEM without FBS or antibiotics) are added to either the apical (AP) or basolateral (BL) side of the monolayers. Peptidase substrates are obtained from Calbiochem, Inc. (Division of EMD Biosciences, Inc., San Diego, Calif.). Cells are incubated for 2 hrs at 37° C. in a 5% CO₂/95% air atmosphere. Both the apical and basolateral media is then measured for its specific enzyme activity according to the manufacturer's instruction. Cleavage is assessed by detecting fluorescence of the substrates, which reflects cleavage because it separates of the quenching agent from the fluorescent agent present on the substrate, which separation allows fluorescence to be detected.

6.1.2. Production of Delivery Construct

The ntPE-hGH carrier construct and human growth hormone binding protein (see, e.g., Leung et al., 1987, 330: 537-43 for human growth hormone binding protein sequence information;) were incubated together overnight (16 hours) at 4° C. to produce a ntPE-hGH-hGHBP carrier construct complex. The human growth hormone binding protein (hGHGP) was obtained from Cell Sciences (Canton, Mass.; Product No. CRH202C). Alternately, hGHBP can be recombinantly expressed using standard techniques known to one of skill in the art.

Two different samples of ntPE-hGH-hGHBP were prepared for in vivo studies as described below. The first contained 2 mg/ml ntPE-hGH carrier construct and 0.888 mg/ml hGHBP solution in a final volume of 1.5 ml. The second contained 2 mg/ml ntPE-hGH carrier construct and 1 mg/ml hGHBP solution in a final volume of 0.5 ml.

6.1.3. Detection of Growth Hormone Non-covalently Bound to Growth Hormone Binding Protein in Tissue bv Histological Examination

This example describes histological detection in tissues of a representative macromolecule-binding partner complex for delivery, growth hormone non-covalently bound to growth hormone binding protein. Following administration of the delivery construct, animals are euthanized by, e.g., CO₂ asphyxiation and exanguinated by cardiac puncture. Specific tissues (lymph nodes, trachea, brain, spleen liver, GI tract) are removed, briefly rinsed in PBS to remove any residual blood and frozen in OCT. Sections (5 microns thick) are placed onto slides. Slides are fixed in acetone for 10 min and rinsed with PBS. Slides are incubated with 3% peroxidase for 5 min. Slides are then blocked with protein for an additional 5 min. Primary growth hormone antibody and primary growth hormone binding protein antibody are incubated onto slides for 30 min at a 1:100 dilution followed by PBS washes. Biotin-labeled secondary antibody specific for the growth hormone antibody and an alkaline phosphatase (AP)-conjugated secondary antibody specific for the growth hormone binding protein antibody are then incubated for approximately 15 minutes followed by PBS washes. Streptavidin HRP label is incubated onto slides for 15 min followed by PBS washes. HRP Chromagen is applied for 5 min followed by several rinses in distilled H₂O. A chromogenic substrate for AP could also be applied and allowed sufficient time and proper conditions to react prior to washing. Finally, the slides are counterstained with hematoxylin for 1 min, coverslipped, and examined for the presence of GH and growth hormone binding protein.

6.1.4. Delivery Construct in an In Vivo System

This example describes use of the delivery construct in a mouse model, showing effective transport and cleavage of the carrier construct in vivo and the bioactivity of the hGH-hGHBP delivered.

6.1.4.1. Administration of Delivery Construct Comprising HGH-HGHGP

Using an animal feeding needle, 100 μl of the first hGH-hGHGP delivery construct described above, containing 100 μg total protein (diluted in PBS containing 1 mg/ml bovine serum albumin), was orally delivered to three groups of four female BALB/c mice, 5-6 weeks of age (Charles River Laboratories, Wilmington, Mass.). Prior to serum collection, mice were anesthetized by an intraperitoneal injection of 75 mg/kg ketamine and 7.5 mg/kg xylazine. Whole blood was collected via the retro-orbital route with heparinized capillary tubes. Final blood collection was collected via cardiac puncture.

6.1.4.2. Pharmacokinetics of HGH-HGHGP Delivery Construct

To assess the pharmacokinetics of an exemplary macromolecule delivered with a delivery construct, ELISA assays were used to measure serum concentrations of hGHBP at defined timepoints following administration. The serum concentration data thus obtained was used to assess the delivery of hGHBP in complex with ntPE-hGH. The ELISA assays were performed with a commercial hGHBP ELISA kit (Diagnostic Systems Laboratories; Webster, Tex.) according to the manufacturer's instructions.

The ELISA assay was used to determine the concentration of hGHBP in mouse serum 30, 45, 60, 75, and 90 minutes following oral administration. The results of this experiment are presented, in part, as Table 4, below. Mice from all three groups (A-C) received the same oral gavage at T=0 but were used on different schedules to obtain serum samples for hGHBP analysis: Group A=30, 45 and 60 minutes serum collections, Group B=45, 60 and 75 minutes collections, and Group C=60, 75 and 90 minute collections. This method allowed three sequential serum collections for sets of four mice in each group. As shown Table 4, hGHBP serum levels were first detectable in mouse serum 60 minutes following oral administration with increasing levels observed at 75 and 90 minutes. Thus, this Example demonstrates that noncovalent complexes can be orally delivered using the constructs of the present invention.

TABLE 4

| | | Time (min) | | | | |
|---|---|---|---|---|---|---|
| | | 30 | 45 | 60 | 75 | 90 |
| | | hGH-BP | hGH-BP | hGH-BP | hGH-BP | hGH-BP |
| | Sample ID/Date | (ng/ml) | (ng/ml) | (ng/ml) | (ng/ml) | (ng/ml) |
| 1 | Mouse 1, Group A | 0.00 | 0.00 | 4.20 | n/a | n/a |
| 2 | Mouse 2, Group A | 0.00 | 0.00 | 4.90 | n/a | n/a |
| 3 | Mouse 3, Group A | 0.00 | 0.00 | 4.00 | n/a | n/a |
| 4 | Mouse 4, Group A | 0.00 | 0.00 | 6.10 | n/a | n/a |
| 5 | Mouse 5, Group B | n/a | 0.00 | 2.30 | 3.60 | n/a |
| 6 | Mouse 6, Group B | n/a | 0.00 | 0.00 | 4.60 | n/a |
| 7 | Mouse 7, Group B | n/a | 0.00 | 0.00 | 2.70 | n/a |
| 8 | Mouse 8, Group B | n/a | 0.00 | 0.00 | 3.60 | n/a |
| 9 | Mouse 9, Group C | n/a | n/a | 151.70 | 183.10 | 315.00 |
| 10 | Mouse 10, Group C | n/a | n/a | 0.00 | 0.00 | 3.90 |
| 11 | Mouse 11, Group C | n/a | n/a | 279.60 | 390.00 | 526.80 |
| 12 | Mouse 12, Group C | n/a | n/a | 0.00 | 2.20 | 5.60 |
| | Avg | 0.00 | 0.00 | 37.73 | 73.73 | 212.83 |
| | SEM | 0.00 | 0.00 | 24.18 | 47.14 | 110.57 |

6.1.4.3. Assays Demonstrating Activity of a hGH Following Delivery with a Delivery Construct

This example describes analysis of the biological effects of an exemplary macromolecule, hGHBP, delivered as a complex with hGH using the ntPE-hGH-hGHBP delivery construct in an in vivo system. In brief, insulin-like growth factor I-binding protein 3 (IGF-I-BP3), growth hormone (GH) receptor and insulin-like growth factor I (IGF-I) expression levels are assessed in liver tissue obtained from mice following administration of either the ntPE-hGH ot ntPE-hGH-hGHGP delivery construct to demonstrate oral delivery of biologically active hormone or hormone complex. Liver RNAtranscripts are analyzed because of the well-characterized effects of GH and/or GHBP on IGF-I-BP3 and GH receptor levels. In particular, functional activation of the GH receptor following binding by GH is known to result in upregulation of IGF-I-BP3 and downregulation of GH receptor expression. Of these, upregulation of IGF-I-BP3 mRNA expression is believed to be the most reliable indicator of GH receptor activation. See, e.g., Sondergaard et al., 2003, Am J Physiol Endocrinol Metab 285:E427-32.

Thus, Quantitative Real Time PCR is used to detect and quantify the amount of IGF-I-BP3, GH receptor, IGF-I, and glyceraldehyde-3-phosphate dehydrogenase (GAPDH) mRNA in approximately 30 mg of mouse liver tissue prepared as described above. Collected liver tissue is stored at −70° C. until further processing. Real-time detection of PCR is performed using the Applied Biosystems 7300 Real Time PCR system (Applied Biosystems, Foster City, Calif.). Total RNA from mouse liver is isolated according to the RNeasy Protect Mini Kit (Qiagen). Total RNA is used to generate cDNA for oligo dT oligodeoxynucleotide primer (T12-18) following the protocol for Omniscript Reverse Transcriptase (Qiagen). The primers used to amplify the cDNA are designed using Primer Express software (Applied Biosystems), synthesized by Operon (Alameda, Calif.).

Equal amounts of cDNA are used in duplicate and amplified with the SYBR Green I Master Mix (Applied Biosystems). The thermal cycling parameters are as follows: thermal activation for 10 min at 95° C., and 40 cycles of PCR (melting for 15 s at 95° C. and annealing/extension for 1 min at 60° C.). A standard curve is constructed with a dilution curve (1:10, 1:100, 1:500, 1:1,000, 1:2,000) of total RNA from a control mouse liver sample. A “no template control” is included with each PCR. Amplification efficiencies were validated and normalized against GAPDH. Correct PCR product size is confirmed by electrophoresis through a 1% agarose gel stained with ethidium bromide. Purity of the amplified PCR products is determined by a heat-dissociation protocol.

6.2. ntPE-Protein G Antibody Delivery Construct

6.2.1. Construction of ntPE-Protein G Antibody Delivery Construct

ntPE-Protein G carrier constructs comprise sequences encoding Domains I and II of ntPE (amino acid residues 26-372 as shown in FIG. 1) and the Fc-binding domain of Protein G (SEQ ID NO:24). The Fc-binding domain of Protein G is attached to the C-terminus of ntPE. BL21(DE3)pLysS competent cells transfected with ntPE-Protein G expression vector were grown in 2×LB broth containing 50 μg/ml ampicillin at 37° C. The expression of recombinant ntPE Protein G was induced at OD₆₀₀=0.8 with 1 mM isopropyl b-D-thiogalactoside. The cells were harvested 4 hrs after induction and the inclusion bodies were extracted and solubilized with 6 M Guanidine and 65 mM DTT. The protein was renaturized on size-exclusion column and purified by sequential column chromatography using Q sepharose HP and Sephadex 200. Then, a final concentration of 0.4 mg/ml of ntPE Protein G was mixed with 0.8 mg/ml of human IgG (molar ratio:2:1) in PBS for 2 hrs at room temperature.

6.2.2. Administration of ntPE-Protein G Antibody Delivery Construct to Mice

100 μg of the suspension of protein mixture was administered by oral gavage to BALB/c mice in 250 μl of PBS with 1 mg/ml of BSA as a carrier. Serum samples, prepared from blood collected at the time points identified in FIG. 2, were analyzed for the presence of human IgG by ELISA.

6.2.3. Measurement of Human IgG in Mouse Serum Using Monoclonal Antibodies

Human IgG in mouse serum samples were measured by ELISA. The employed Human IgG ELISA method was developed by Trinity Biosystems and was conducted in accordance with SOP-032. Costar 9018 E.I.A./R.I.A. 96-well plates were coated overnight with about 300 ng/well of mouse anti-human IgG (Abcam, Cat. No. ab7497) in 0.2M NaHCO₃-Na₂CO₃, pH 9.4. Each 96-well plate was washed four times with PBS containing 0.05% Tween 20-0.01% thimerosal (wash buffer); blocked for 1 h with 200 μl/well of PBS/Tween 20 containing 0.5% BSA-0.01% thimerosal (assay buffer). Purified Human IgG (Antibodies Inc., Cat. No. 43-636) diluted in assay buffer was used as the standard curve. Standard curve was prepared by adding 10 μl of the 1.0 mg/ml Human IgG to 990 μl assay buffer (1:100), mixing well and moving 10 μl to 990 μl assay buffer (1:100). This solution was used as the first point for the standard curve. For each plate, 0.5 ml was moved to 0.5 ml assay buffer, and did a 1:2 serial dilution. The 10points are of the standard curve were: 100, 50, 25, 12.5, 6.25, 3.125, 1.56, 0.78, 0.39, and 0.195 ng/well. Serum samples were diluted at 1:10in assay buffer. Each plate was washed again, and standard curve and samples were loaded in 100μ/well triplicates onto a 96-well plate, and incubated for 3 h to detect Human IgG in serum samples. Each 96-well plate was then washed four times with wash buffer, and added 100 μl/well of mouse anti-human IgG-biotin (Zymed, Cat. No. 05-4240) at 1:1000 dilutions and incubated for 2 h. Each 96-well plate was then washed four times with wash buffer, and added 100 μl/well of horseradish peroxidase (HRP) conjugated ExtrAvidin (Sigma, Cat. No. E-2886) at 1:2000 dilutions and incubated for 1 h. All incubation and coating steps were performed at room temperature on a shaker at 6 RPM. Each 96-well plate was then washed four times with wash buffer, and the HRP substrate, TMB (3,3′, 5,5′tetramethylbenzidine), used to quantify bound antibody, was measured at 450 nm.

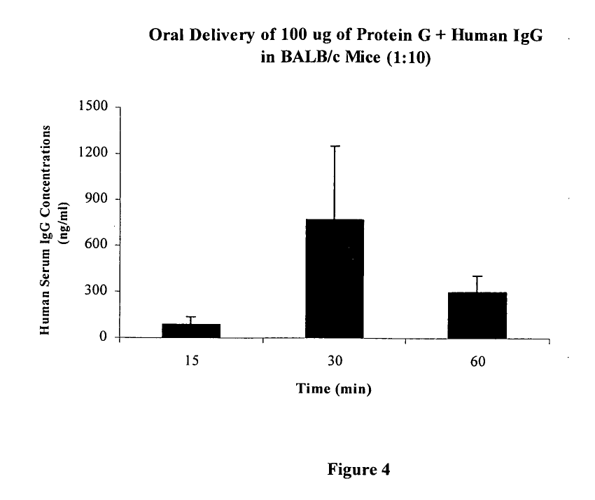

ELISA results are reported as the averages of the triplicate OD (450 nm) value of each sample. See FIG. 4.

6.3. ntPE-Protein A Antibody Delivery Construct

6.3.1. Construction of ntPE-Protein A Antibody Delivery Construct

ntPE-Protein A carrier constructs comprise sequences encoding Domains I and II of ntPE (amino acid residues 26-372 as shown in FIG. 1) and a Protein A antibody-binding fragment (SEQ ID NO:25). The Protein A antibody-binding fragment is attached to the C-terminus of ntPE. BL21(DE3)pLysS competent cells are transfected with ntPE-Protein A expression vector. The transfected cells are grown in 2×LB broth containing 50 μg/ml ampicillin at 37° C. The expression of recombinant ntPE Protein A is induced at OD₆₀₀=0.8 with 1 mM isopropyl b-D-thiogalactoside. The cells are harvested 4 hrs after induction and the inclusion bodies is extracted and solubilized with 6 M Guanidine and 65 mM DTT. The protein is renaturized on size-exclusion column and purified by sequential column chromatography using Q sepharose HP and Sephadex 200. Then, a final concentration of 0.4 mg/ml of ntPE-Protein A is mixed with 0.8 mg/ml of human IgG (molar ratio:2:1) in PBS for 2 hrs at room temperature.

6.3.2. Administration of Protein A-Antibody Delivery Construct to Mice

100 μg of the protein solution is administered by oral gavage to BALB/c mice in 250 μl of PBS with 1 mg/ml of BSA as a carrier. Serum samples, prepared from blood collected at various time points, are analyzed for the presence of human IgG by ELISA.

6.3.3. Measurement of Human IgG in Mouse Serum Using Monoclonal Antibodies

Human IgG in mouse serum samples are measured by the ELISA described in Section 6.2.3, supra.

6.4. ntPE-FcRn Antibody Deliverv Construct

6.4.1. Construction of FcRn-Antibody Delivery Construct

ntPE-FcRn carrier construct comprises sequences encoding Domains I and II of ntPE (amino acid residues 26-372 as shown in FIG. 1) and human FcRn (SEQ ID NO:26; Mikulska et al., 2000, Eur. J. immunogenet 27(4): 231-240). The human FcRn is attached to the C-terminus of ntPE. Some of the carrier constructs comprise a cleavable linker between the ntPE sequences and the FcRn sequences. In particular, some of the constructs comprise one of the following cleavable linkers: RQPRGGL (SEQ ID NO:30), GGLRQPR (SEQ ID NO:31), RQPREGR (SEQ ID NO.:32), RQPRVGR (SEQ ID NO.:33), and RQPRARR (SEQ ID NO.:34). BL21(DE3)pLysS competent cells are transfected with ntPE-FcRn expression vector. The transfected cells are grown in 2×LB broth containing 50 μg/ml ampicillin at 37° C. The expression of recombinant ntPE-FcRn is induced at OD₆₀₀=0.8 with 1 mM isopropyl b-D-thiogalactoside. The cells are harvested 4 hrs after induction and the inclusion bodies are extracted and solubilized with 6M Guanidine and 65 mM DTT. In an alternate approach, ntPE-FcRN is expressed in a soluble, folded form from a mammalian cell expression system such as CHO or BHK cells. The protein is renaturized on size-exclusion column and purified by sequential column chromatography using Q sepharose HP and Sephadex 200. Then, a final concentration of 0.4 mg/ml of ntPE-FcRn is mixed with 0.8 mg/ml of human IgG (molar ratio:2:1) in PBS for 2 hrs at room temperature. In particular, a final concentration of 0.4 mg/ml of ntPE-FcRn is mixed with 0.8 mg/ml of Avastin (molar ratio:2:1) or 0.8 mg/ml of Rituxan in PBS for 2 hrs at room temperature.

6.4.2. Administration of ntPE-FcRn-Antibody Delivery Construct to Mice

100 μg of the suspension of protein mixture is administered by oral gavage to BALB/c mice in 250 μl of PBS with 1 mg/ml of BSA as a carrier. Serum samples, prepared from blood collected at various time points, are analyzed for the presence of human IgG by ELISA.

6.4.3. Measurement of Human IgG in Mouse Serum Using Monoclonal Antibodie

Human IgG in mouse serum samples are measured by the ELISA described in Section 6.2.3, supra.

6.5. ntPE-FcγRIII Antibody Delivery Construct

6.5.1. Construction of FcR-Antibody Deliverv Construct

ntPE-FcγRIII carrier construct comprises sequences encoding Domains I and II of ntPE (amino acid residues 26-372 as shown in FIG. 1) and human FcγRIII (SEQ ID NO: 27; Radaev et al., 2001, Journal of Biological Chemistry 276: 16469) or human FcγRIII-beta (SEQ ID NO:28), or an antibody-binding domain of human FcγRIII-beta (SEQ ID NO:29). The human FcγRIII is attached to the C-terminus of ntPE. Some of the carrier constructs comprise a cleavable linker between the ntPE sequences and the FcγRIII sequences. In particular, some of the constructs comprise one of the following cleavable linkers: RQPRGGL (SEQ ID NO.:30), GGLRQPR (SEQ ID NO.:3 1), RQPREGR (SEQ ID NO.:32), RQPRVGR (SEQ ID NO.:33), and RQPRARR (SEQ ID NO.:34). BL21(DE3)pLysS competent cells are transfected with ntPE-FcγRIII expression vector. The transfected cells are grown in 2×LB broth containing 50 μg/ml ampicillin at 37° C. The expression of recombinant ntPE-FcγRIII is induced at OD₆₀₀=0.8 with 1 mM isopropyl b-D-thiogalactoside. The cells are harvested 4 hrs after induction and the inclusion bodies are extracted and solubilized with 6M Guanidine and 65 mM DTT. The protein is renaturized on size-exclusion column and purified by sequential column chromatography using Q sepharose HP and Sephadex 200. In an alternate approach, ntPE-FcγRIII is expressed in a soluble, folded form from a mammalian cell expression system such as CHO or BHK cells. Then, a final concentration of 0.4 mg/ml of ntPE-FcγRIII is mixed with 0.8 mg/ml of human IgG (molar ratio:2:1) in PBS for 2 hrs at room temperature. In particular, a final concentration of 0.4 mg/ml of ntPE-FcγRIII is mixed with 0.8 mg/ml of Avastin (molar ratio:2:1) or 0.8 mg/ml of Rituxan in PBS for 2 hrs at room temperature.

6.5.2. Administration of ntPE-FcγRIII Antibody Deliverv Construct to Mice

100 μg of the suspension of protein mixture is administered by oral gavage to BALB/c mice in 250 μl of PBS with 1 mg/ml of BSA as a carrier. Serum samples, prepared from blood collected at various time points, are analyzed for the presence of human IgG by ELISA.

6.5.3. Measurement of Human IgG in Mouse Serum Using Monoclonal Antibodie

Human IgG in mouse serum samples are measured by the ELISA described in Section 6.2.3, supra.

6.6. Delivery of an Exemplary Complex in an In Vivo System

This example describes successful oral delivery of a noncovalent complex to an exemplary model organism with an exemplary delivery construct. In this example, the exemplary noncovalent complex delivered is aggregated insulin. The receptor binding and translocation portions of the delivery construct are covalently attached to an insulin molecule. The insulin molecule is self-associated with other insulin molecules to form a noncovalent complex. Thus, in this example, the binding partner and the macromolecule are the same protein, insulin.

First, 100 units of regular insulin (Novo Nordisk) in 2 mls buffer were adjusted to pH 5.0 with MES buffer and zinc chloride was added to a final concentration of 1 mM. The insulin was then incubated at room temperature for 10 minutes to allow the insulin molecules to aggregate.

Next, either 2 mg (1×) or 4 mg (2×) ntPE was added to 50 Units aggregated insulin to test the effects of different ratios of polypeptide to particle. 100 mg ethylene diimine carbodiimide was then added to the reaction mixture to cross-link the insulin aggregates and nt-PE, then the reaction was incubated on ice for 30 minutes. The 1×and 2×delivery constructs thus made were then dialyzed overnight against pH 7 phosphate-buffered saline.

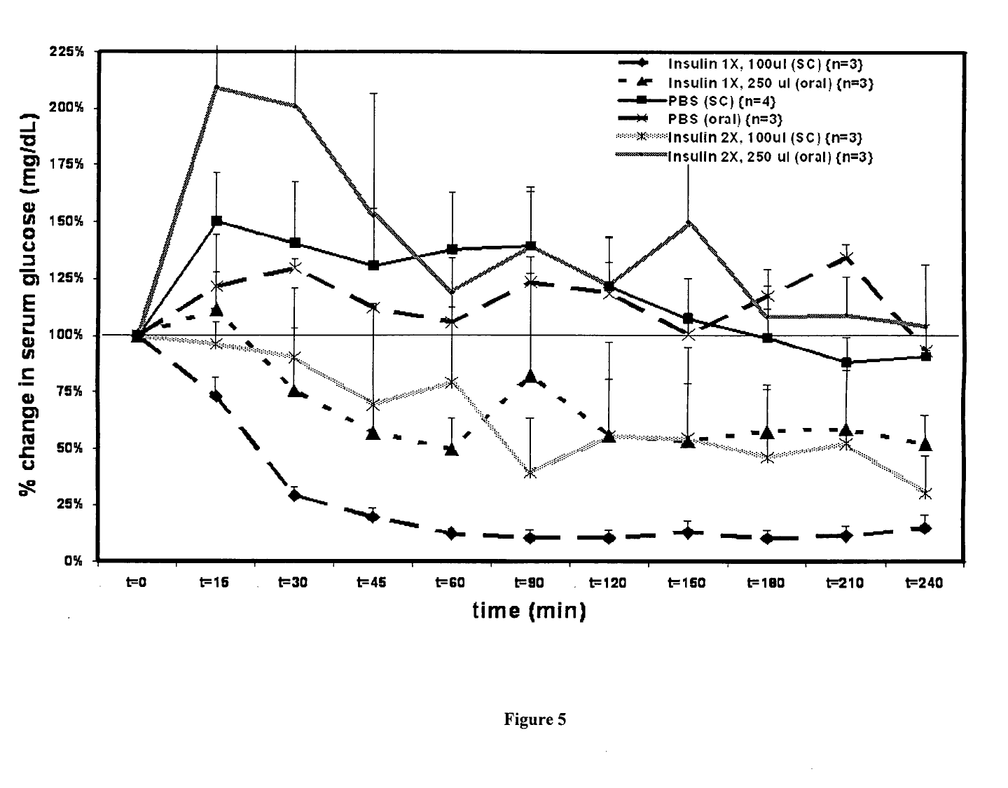

To assess the activity of the delivery constructs, either 100 μl by subcutaneous injection or 250 μl by oral gavage of the 1×x delivery construct, the 2×delivery construct, or PBS as negative control was administered to fasted female STZ BALB/c mice. Serum blood glucose was monitored every 15 minutes for the first hour, then every 30 minutes thereafter, to assess the effects of the insulin aggregates delivered with the delivery constructs. Experiments were performed in triplicate and results are presented as an average of the three experiments. The results of the experiment are presented as FIG. 5.

As shown in FIG. 5, the 1×delivery construct administered subcutaneously resulted in the greatest decrease in blood glucose concentration. Similarly, oral administration of the 1×delivery construct also resulted in a substantial decrease in blood glucose concentration. Thus, the 1×delivery construct effectively delivered the aggregated insulin in a bioactive form to the tested animals. As discussed below, the data also suggest that the 2×delivery construct also delivered aggregated insulin. The 2×delivery construct did not work as well as the 1×delivery construct, suggesting that routine optimization of the ratio of polypeptide carrier to complex can increase or optimize the efficiency of particle delivery. Finally, the PBS negative control demonstrates that the stress of oral gavage (and, to a lesser extent, subcutaneous injection) of mice results in release of glucose from energy reserves. Thus, the increased glucose concentrations observed following oral administration of the 2×delivery construct can be attributed to this effect. It should be noted that the increase observed from oral administration of the 2×delivery construct was less than that observed for the appropriate negative control, suggesting that the 2×delivery construct was also able to deliver bioactive insulin aggregates to test animals. Thus, these results demonstrate that the delivery constructs of the invention can be used to deliver an aggregate of a bioactive molecule to the serum of a representative test animal and that those aggregates can exert a biological effect in the animal once delivered.

The present invention provides, inter alia, delivery constructs and methods of inducing an immune response in a subject. While many specific examples have been provided, the above description is intended to illustrate rather than limit the invention. Many variations of the invention will become apparent to those skilled in the art upon review of this specification. The scope of the invention should, therefore, be determined not with reference to the above description, but instead should be determined with reference to the appended claims along with their full scope of equivalents.

All publications and patent documents cited in this application are incorporated by reference in their entirety for all purposes to the same extent as if each individual publication or patent document were so individually denoted. Citation of these documents is not an admission that any particular reference is “prior art” to this invention.

TABLE 5
Human Peptidases by Class

Aspartic-type peptidases
BAE1_HUMAN (P56817)
BAE2_HUMAN (Q9Y5Z0)
CATD_HUMAN (P07339)
CATE_HUMAN (P14091)
NAP1_HUMAN (O96009)
PEPA_HUMAN (P00790
PEPC_HUMAN (P20142)
RENI_HUMAN (P00797)
VPRT_HUMAN (P10265)
Other Peptidases
FAC2_HUMAN (Q9Y256)
Cysteine-type peptidases
BLMH_HUMAN (Q13867)
CATB_HUMAN (P07858)
CATC_HUMAN (P53634)
CATF_HUMAN (Q9UBX1)
CATH_HUMAN (P09668)
CATK_HUMAN (P43235)
CATL_HUMAN (P07711)
CATO_HUMAN (P43234)
CATS_HUMAN (P25774)
CATW_HUMAN (P56202)
CATZ_HUMAN (Q9UBR2)
CSL2_HUMAN (O60911)
TNAG_HUMAN (Q9UJW2)
CAN1_HUMAN (P07384)
CAN2_HUMAN (P17655)
CAN3_HUMAN (P20807)
CAN5_HUMAN (O5484)
CAN6_HUMAN (Q9Y6Q1)
CAN7_HUMAN (Q9Y6W3)
CAN9_HUMAN (O14815)
CANA_HUMAN (Q9HC96)
CANB_HUMAN (Q9UMQ6)
UBL1_HUMAN (P09936)
UBL3_HUMAN (P15374)
UBL5_HUMAN (Q9Y5K5)
GPI8_HUMAN (Q92643)
LGMN_HUMAN (Q99538)
CFLA_HUMAN (O15519)
I1C_HUMAN (P29466)
ICE2_HUMAN (P42575)
ICE3_HUMAN (P42574)
ICE4_HUMAN (P49662)
ICE5_HUMAN (P51878)
ICE6_HUMAN (P55212)
ICE7_HUMAN (P55210)
ICE8_HUMAN (Q14790)
ICE9_HUMAN (P55211)
ICEA_HUMAN (Q92851)
ICEE_HUMAN (P31944)
MLT1_HUMAN (Q9UDY8)
PGPI_HUMAN (Q9NXJ5)
FAFX_HUMAN (Q93008)
FAFY_HUMAN (O00507)
UB10_HUMAN (Q14694)
UB11_HUMAN (P51784)
UB12_HUMAN (O75317)
UB13_HUMAN (Q92995)
UB14_HUMAN (P54578)
UB15_HUMAN (Q9Y4E8)
UB16_HUMAN (Q9Y5T5)
UB18_HUMAN (Q9UMW8)
UB19_HUMAN (O94966)
UB20_HUMAN (Q9Y2K6)
UB21_HUMAN (Q9UK80)
UB22_HUMAN (Q9UPT9)
UB24_HUMAN (Q9UPU5)
UB25_HUMAN (Q9UHP3)
UB26_HUMAN (Q9BXU7)
UB28_HUMAN (Q96RU2)
UB29_HUMAN (Q9HBJ7)
UB32_HUMAN (Q8NFA0)
UB33_HUMAN (Q8TEY7)
UB35_HUMAN (Q9P2H5)
UB36_HUMAN (Q9P275)
UB37_HUMAN (Q86T82)
UB38_HUMAN (Q8NB14)
UB40_HUMAN (Q9NVE5)
UB42_HUMAN (Q9H9J4)
UB44_HUMAN (Q9H0E7)
UB46_HUMAN (P62068)
UBP1_HUMAN (O94782)
UBP2_HUMAN (O75604)
UBP3_HUMAN (Q9Y614)
UBP4_HUMAN (Q13107)
UBP5_HUMAN (P45974)
UBP6_HUMAN (P35125)
UBP7_HUMAN (Q93009)
UBP8_HUMAN (P40818)
GGH_HUMAN (Q92820)
SEN1_HUMAN (Q9P0U3)
SEN3_HUMAN (Q9H4L4)
SEN5_HUMAN (Q96H10)
SEN6_HUMAN (Q9GZR1)
SEN7_HUMAN (Q9BQF6)
SEN8_HUMAN (Q96LD8)
SNP2_HUMAN (Q9HC62)
ESP1_HUMAN (Q14674)
Metallopeptidases
AMPB_HUMAN (Q9H4A4)
AMPE_HUMAN (Q07075)
AMPN_HUMAN (P15144)
ART1_HUMAN (Q9NZ08)
LCAP_HUMAN (Q9UIQ6)
LKHA_HUMAN (P09960)
PSA_HUMAN (P55786)
RNPL_HUMAN (Q9HAU8)
THDE_HUMAN (Q9UKU6)
ACET_HUMAN (P22966)
ACE_HUMAN (P12821)
MEPD_HUMAN (P52888)
NEUL_HUMAN (Q9BYT8)
PMIP_HUMAN (Q99797)
MM01_HUMAN (P03956)
MM02_HUMAN (P08253)
MM03_HUMAN (P08254)
MM07_HUMAN (P09237)
MM08_HUMAN (P22894)
MM09_HUMAN (P14780)
MM10_HUMAN (P09238)
MM11_HUMAN (P24347)
MM12_HUMAN (P39900)
MM13_HUMAN (P45452)
MM14_HUMAN (P50281)
MM15_HUMAN (P51511)
MM16_HUMAN (P51512)
MM17_HUMAN (Q9ULZ9)
MM19_HUMAN (Q99542)
MM20_HUMAN (O60882)
MM21_HUMAN (Q8N119)
MM24_HUMAN (Q9Y5R2)
MM25_HUMAN (Q9NPA2)
MM26_HUMAN (Q9NRE1)
MM28_HUMAN (Q9H239)
BMP1_HUMAN (P13497)
MEPA_HUMAN (Q16819)
MEPB_HUMAN (Q16820)
AD02_HUMAN (Q99965)
AD07_HUMAN (Q9H2U9)
AD08_HUMAN (P78325)
AD09_HUMAN (Q13443)
AD10_HUMAN (O14672)
AD11_HUMAN (O75078)
AD12_HUMAN (O43184)
AD15_HUMAN (Q13444)
AD17_HUMAN (P78536)
AD18_HUMAN (Q9Y3Q7)
AD19_HUMAN (Q9H013)
AD20_HUMAN (O43506)
AD21_HUMAN (Q9UKJ8)
AD22_HUMAN (Q9P0K1)
AD28_HUMAN (Q9UKQ2)
AD29_HUMAN (Q9UKF5)
AD30_HUMAN (Q9UKF2)
AD33_HUMAN (Q9BZ11)
AT10_HUMAN (Q9H324)
AT12_HUMAN (P58397)
AT14_HUMAN (Q8WXS8)
AT15_HUMAN (Q8TE58)
AT16_HUMAN (Q8TE57)
AT17_HUMAN (Q8TE56)
AT18_HUMAN (Q8TE60)
AT19_HUMAN (Q8TE59)
AT20_HUMAN (P59510)
ATS1_HUMAN (Q9UHI8)
ATS2_HUMAN (O95450)
ATS3_HUMAN (O15072)
ATS4_HUMAN (O75173)
ATS5_HUMAN (Q9UNA0)
ATS6_HUMAN (Q9UKP5)
ATS7_HUMAN (Q9UKP4)
ATS8_HUMAN (Q9UP79)
ATS9_HUMAN (Q9P2N4)
ECE1_HUMAN (P42892)
ECE2_HUMAN (O60344)
ECEL_HUMAN (O95672)
KELL_HUMAN (P23276)
NEP_HUMAN (P08473)
PEX_HUMAN (P78562)
CBP1_HUMAN (P15085)
CBP2_HUMAN (P48052)
CBP4_HUMAN (Q9UI42)
CBP5_HUMAN (Q8WXQ8)
CBP6_HUMAN (Q8N4T0)
CBPB_HUMAN (P15086)
CBPC_HUMAN (P15088)
CBPD_HUMAN (O75976)
CBPE_HUMAN (P16870)
CBPM_HUMAN (P14384)
CBPN_HUMAN (P15169)
CPX2_HUMAN (Q8N436)
CPXM_HUMAN (Q96SM3)
IDE_HUMAN (P14735)
MPPA_HUMAN (Q10713)
MPPB_HUMAN (O75439)
NRDC_HUMAN (O43847)
UCR1_HUMAN (P31930)
UCR2_HUMAN (P22695)
AMPL_HUMAN (P28838)
PEL1_HUMAN (Q8NDH3)
DNPE_HUMAN (Q9ULA0)
MDP1_HUMAN (P16444)
CGL1_HUMAN (Q96KP4)
CGL2_HUMAN (Q96KN2)
ACY1_HUMAN (Q03154)
GCP_HUMAN (Q9NPF4)
AMP1_HUMAN (P53582)
PEPD_HUMAN (P12955)
XPP2_HUMAN (O43895)
AMP2_HUMAN (P50579)
P2G4_HUMAN (Q9UQ80)
FOH1_HUMAN (Q04609)
NLD2_HUMAN (Q9Y3Q0)
NLDL_HUMAN (Q9UQQ1)
TFR1_HUMAN (P02786)
TFR2_HUMAN (Q9UP52)
AF31_HUMAN (O43931)
AF32_HUMAN (Q9Y4W6)
SPG7_HUMAN (Q9UQ90)
YME1_HUMAN (Q96TA2)
PAPA_HUMAN (Q13219)
FAC1_HUMAN (O75844)
DPP3_HUMAN (Q9NY33)
MS2P_HUMAN (O43462)
Serine-type peptidases
ACRL_HUMAN (P58840)
ACRO_HUMAN (P10323)
APOA_HUMAN (P08519)
BSS4_HUMAN (Q9GZN4)
C1R_HUMAN (P00736)
C1S_HUMAN (P09871)
CAP7_HUMAN (P20160)
CATG_HUMAN (P08311)
CFAB_HUMAN (P00751)
CFAD_HUMAN (P00746)
CFA1_HUMAN (P05156)
CLCR_HUMAN (Q99895)
CO2_HUMAN (P06681)
CORI_HUMAN (Q9Y5Q5)
CRAR_HUMAN (P48740)
CTRB_HUMAN (P17538)
CTRL_HUMAN (P40313)
DES1_HUMAN (Q9UL52)
EL1_HUMAN (Q9UNI1)
EL2A_HUMAN (P08217)
EL2B_HUMAN (P08218)
EL3A_HUMAN (P09093)
EL3B_HUMAN (P08861)
ELNE_HUMAN (P08246)
ENTK_HUMAN (P98073)
FA10_HUMAN (P00742)
FA11_HUMAN (P03951)
FA12_HUMAN (P00748)
FA7_HUMAN (P08709)
FA9_HUMAN (P00740)
GRAA_HUMAN (P12544)
GRAB_HUMAN (P10144)
GRAH_HUMAN (P20718)
GRAK_HUMAN (P49863)
GRAM_HUMAN (P51124)
HATT_HUMAN (O60235)
HEPS_HUMAN (P05981)
HGFA_HUMAN (Q04756)
HGFL_HUMAN (P26927)
HGF_HUMAN (P14210)
HPTR_HUMAN (P00739)
HPT_HUMAN (P00738)
KAL_HUMAN (P03952)
KLK1_HUMAN (P06870)
KLK2_HUMAN (P20151)
KLK3_HUMAN (P07288)
KLK4_HUMAN (Q9Y5K2)
KLK5_HUMAN (Q9Y337)
KLK6_HUMAN (Q92876)
KLK7_HUMAN (P49862)
KLK8_HUMAN (O60259)
KLK9_HUMAN (Q9UKQ9)
KLKA_HUMAN (O43240)
KLKB_HUMAN (Q9UBX7)
KLKC_HUMAN (Q9UKR0)
KLKD_HUMAN (Q9UKR3)
KLKE_HUMAN (Q9P0G3)
KLKF_HUMAN (Q9H2R5)
LCLP_HUMAN (P34168)
MAS2_HUMAN (O00187)
MCT1_HUMAN (P23946)
NETR_HUMAN (P56730)
PLMN_HUMAN (P00747)
PR27_HUMAN (Q9BQR3)
PRN3_HUMAN (P24158)
PRTC_HUMAN (P04070)
PRTZ_HUMAN (P22891)
PS23_HUMAN (O95084)
PSS8_HUMAN (Q16651)
ST14_HUMAN (Q9Y5Y6)
TEST_HUMAN (Q9Y6M0)
THRB_HUMAN (P00734)
TMS2_HUMAN (O15393)
TMS3_HUMAN (P57727)
TMS4_HUMAN (Q9NRS4)
TMS5_HUMAN (Q9H3S3)
TMS6_HUMAN (Q8IU80)
TPA_HUMAN (P00750)
TRB1_HUMAN (Q15661)
TRB2_HUMAN (P20231)
TRY1_HUMAN (P07477)
TRY2_HUMAN (P07478)
TRY3_HUMAN (P35030)
TRYA_HUMAN (P15157)
TRYD_HUMAN (Q9BZJ3)
TRYG_HUMAN (Q9NRR2)
TS50_HUMAN (Q9UI38)
UROK_HUMAN (P00749)
HRA1_HUMAN (Q92743)
HRA2_HUMAN (O43464)
HRA3_HUMAN (P83110)
HRA4_HUMAN (P83105)
FURI_HUMAN (P09958)
MSIP_HUMAN (Q14703)
NEC1_HUMAN (P29120)
NEC2_HUMAN (P16519)
PCK5_HUMAN (Q92824)
PCK6_HUMAN (P29122)
PCK7_HUMAN (Q16549)
PCK9_HUMAN (Q8NBP7)
TPP2_HUMAN (P29144)
PPCE_HUMAN (P48147)
DPP4_HUMAN (P27487)
DPP6_HUMAN (P42658)
SEPR_HUMAN (Q12884)
ACPH_HUMAN (P13798)
CPVL_HUMAN (Q9H3G5)
PRTP_HUMAN (P10619)
RISC_HUMAN (Q9HB40)
CLPP_HUMAN (Q16740)
LONM_HUMAN (P36776)
SPC3_HUMAN (Q9BY50)
SPC4_HUMAN (P21378)
DPP2_HUMAN (Q9UHL4)
PCP_HUMAN (P42785)
TSSP_HUMAN (Q9NQE7)
HYEP_HUMAN (P07099)
TPP1_HUMAN (O14773)
RHB1_HUMAN (O75783)
RHB2_HUMAN (Q9NX52)
RHB4_HUMAN (P58872)
Threonine-type peptidases
PS7L_HUMAN (Q8TAA3)
PSA1_HUMAN (P25786)
PSA2_HUMAN (P25787)
PSA3_HUMAN (P25788)
PSA4_HUMAN (P25789)
PSA5_HUMAN (P28066)
PSA6_HUMAN (P60900)
PSA7_HUMAN (O14818)
PSB1_HUMAN (P20618)
PSB2_HUMAN (P49721)
PSB3_HUMAN (P49720)
PSB4_HUMAN (P28070)
PSB5_HUMAN (P28074)
PSB6_HUMAN (P28072)
PSB7_HUMAN (Q99436)
PSB8_HUMAN (P28062)
PSB9_HUMAN (P28065)
PSBA_HUMAN (P40306)

1. A delivery construct, comprising a carrier construct non-covalently bound to a binding partner, wherein said carrier construct comprises:
a) a receptor binding domain,
b) a transcytosis domain, and
c) a macromolecule to which the binding partner non-covalently binds, wherein the binding partner binds to the macromolecule with a Kₐ that is at least about 10⁴ M⁻¹.

2. The delivery construct of claim 1, wherein the carrier construct further comprises a cleavable linker, wherein cleavage at said cleavable linker separates said macromolecule from the remainder of said carrier construct, and wherein said cleavable linker is cleavable by an enzyme that i) exhibits greater activity at a basal-lateral membrane of a polarized epithelial cell than at an apical membrane of the polarized epithelial cell, or ii) exhibits greater activity in the plasma of a subject than at an apical membrane of the polarized epithelial cell of the subject.

3. The delivery construct of claim 1, wherein said binding partner is selected from the group consisting of a nucleic acid, a peptide, a polypeptide, a small organic molecule and a lipid.

4. The delivery construct of claim 3, wherein said polypeptide is selected from the group consisting of a cytokine, cytokine receptor, chemokine, growth factor, growth factor receptor and DNA binding protein.

5. The delivery construct of claim 3, wherein said polypeptide is selected from the group consisting of IGF-I, IGF-II, IGF-III, EGF, IFN-α, IFN-β, IFN-γ, G-CSF, GM-CSF, IL-1, IL-2, IL-3, IL-6, IL-8, IL-12, IL-18, EPO, growth hormone, factor VII, vasopressin, calcitonin, parathyroid hormone, luteinizing hormone-releasing factor, tissue plasminogen activators, proinsulin, insulin, glucocorticoid, amylin, adrenocorticototropin, enkephalin, glucagon-like peptide 1, IGFBP-3, VEGF receptor, FGF-1, FGF-2, and FGF-7.

6. The delivery construct of claim 5, wherein said polypeptide is IGF-I.

7. (canceled)

8. The delivery construct of claim 3, wherein said polypeptide is human growth hormone.

9. The delivery construct of claim 3, wherein said polypeptide is human insulin.

10. The delivery construct of claim 3, wherein said polypeptide is human IFN-α.

11. The delivery construct of claim 3, wherein said polypeptide is human IFN-α2b.

12. The delivery construct of claim 3, wherein said polypeptide is human proinsulin.

13. The delivery construct of claim 5, wherein said polypeptide is IL-2.

14. (canceled)

15. The delivery construct of claim 5, wherein said polypeptide is IL-18.

16. (canceled)

17. The delivery construct of claim 3, wherein said polypeptide is KDR.

18. (canceled)

119. The delivery construct of claim 1, wherein said macromolecule is selected from the group consisting of a nucleic acid, a peptide, a polypeptide, a small organic molecule and a lipid.

20. The delivery construct of claim 19, wherein said polypeptide is selected from the group consisting of a cytokine, cytokine receptor, chemokine, growth factor, growth factor receptor and DNA binding protein.

21. The delivery construct of claim 6, wherein said macromolecule is IGF-I binding protein 3.

22. The delivery construct of claim 7, wherein said macromolecule is human IGF-I binding protein 3.

23. The delivery construct of claim 8, wherein said macromolecule is human growth hormone binding protein.

24. The delivery construct of claim 13, wherein said macromolecule is IL-2 receptor alpha.

25. The delivery construct of claim 14, wherein said macromolecule is human IL-2 receptor alpha 26. The delivery construct of claim 15, wherein said macromolecule is IL-18 binding protein.

27. The delivery construct of claim 16, wherein said macromolecule is human IL-18 binding protein.

28. The delivery construct of claim 17, wherein said macromolecule is the SH2 domain of human Shc-like protein (Sck).

29. The delivery construct of claim 18, wherein said macromolecule is the SH2 domain of human Sck.

30. The delivery construct of claim 2, further comprising a second cleavable linker and a second macromolecule that is selected from the group consisting of a nucleic acid, a peptide, a polypeptide, a lipid, and a small organic molecule, wherein cleavage at said second cleavable linker separates said second macromolecule from the remainder of said construct.

31. The delivery construct of claim 30, wherein said macromolecule is a first polypeptide and said second macromolecule is a second polypeptide.

32. The delivery construct of claim 31, wherein said first polypeptide and said second polypeptide associate to form a multimer.

33. The delivery construct of claim 32, wherein said multimer is a dimer, tetramer, or octamer.

34. The delivery construct of claim 2, wherein said cleavable linker comprises an amino acid sequence that is selected from the group consisting of Ala-Ala-Pro-Phe (SEQ ID NO.:4), Gly-Gly-Phe (SEQ ID NO.:5), Ala-Ala-Pro-Val (SEQ ID NO.:6), Gly-Gly-Leu (SEQ ID NO.:7), Ala-Ala-Leu (SEQ ID NO.:8), Phe-Val-Arg (SEQ ID NO.:9), Val-Gly-Arg (SEQ ID NO.:10).

35. The delivery construct of claim 2, wherein said enzyme that is present at a basal-lateral membrane of a polarized epithelial cell is selected from the group consisting of Cathep sin GI, Chymotryp sin I, Elastase I, Subtilisin AI, Subtilisin AII, Thrombin I, and Urokinase I.

36. The delivery construct of claim 1, wherein said receptor binding domain is selected from the group consisting of a receptor binding domain from Pseudomonas exotoxin A; cholera toxin; botulinum toxin; diptheria toxin; shiga toxin; shiga-like toxin; monoclonal antibodies; polyclonal antibodies; single-chain antibodies; TGF α, EGF; IGF-I; IGF-II; IGF-III; IL-1; IL-2; IL-3; IL-6; MIP-1a; MIP-1b; MCAF; and IL-8.

37. The delivery construct of claim 1, wherein said receptor binding domain binds to a cell-surface receptor that is selected from the group consisting of α2-macroglobulin receptor, epidermal growth factor receptor, transferrin receptor, chemokine receptor, CD25, CD11B, CD11C, CD80, CD86, TNFα receptor, TOLL receptor, M-CSF receptor, GM-CSF receptor, scavenger receptor, and VEGF receptor.

38. The delivery construct of claim 37, wherein said receptor binding domain of Pseudomonas exotoxin A is Domain Ia of Pseudomonas exotoxin A.

39. The delivery construct of claim 37, wherein said receptor binding domain of Pseudomonas exotoxin A has an amino acid sequence that is SEQ ID NO.:1.

40. The delivery construct of claim 1, wherein said transcytosis domain is selected from the group consisting of a transcytosis domain from Pseudomonas exotoxin A, botulinum toxin, diptheria toxin, pertussis toxin, cholera toxin, heat-labile E. coli enterotoxin, shiga toxin, and shiga-like toxin.

41. The delivery construct of claim 40, wherein said transcytosis domain is Pseudomonas exotoxin A transcytosis domain.

42. The delivery construct of claim 41, wherein said Pseudomonas exotoxin A transcytosis domain has an amino acid sequence that is SEQ ID NO.:2.

43. The delivery construct of claim 1, wherein the binding partner binds to the macromolecule with a Kₐ that is at least about 10⁵ M-⁻¹.

44. The delivery construct of claim 1, wherein the binding partner binds to the macromolecule with a Kₐ that is at least about 10⁶ M⁻¹.

45. The delivery construct of claim 1, wherein the binding partner binds to the macromolecule with a Kₐ that is at least about 10⁷ M⁻¹.

46. The delivery construct of claim 1, wherein the binding partner binds to the macromolecule with a Kₐ that is at least about 10⁸ M⁻¹.

47. The delivery construct of claim 1, wherein the binding partner binds to the macromolecule with a Kₐ that is at least about 10⁹ M⁻¹.

48. A cell comprising a first polynucleotide and a second polynucleotide, wherein the first polynucleotide encodes a binding partner and the second polynucleotide encodes a carrier construct comprising:
a) a receptor binding domain,
b) a transcytosis domain, and
c) a macromolecule to which the binding partner non-covalently binds, wherein the binding partner binds to the macromolecule with a Kₐ that is at least about 10⁴ M⁻¹.

49-86. (canceled)

87. A composition comprising a delivery construct of claim 1.

88. The composition of claim 87, wherein said composition further comprises a pharmaceutically acceptable diluent, excipient, vehicle, or carrier.

89. The composition of claim 87, wherein said composition is formulated for nasal or oral administration.

90. A method for delivering a binding partner to a subject, the method comprising contacting an apical surface of a polarized epithelial cell of the subject with a delivery construct comprising a carrier construct non-covalently bound to the binding partner, wherein said carrier construct comprises a receptor binding domain, a transcytosis domain, and a macromolecule to which the binding partner non-covalently binds, and wherein the binding partner binds to the macromolecule with a Kₐ that is at least about 10⁴ M⁻¹, such that the binding partner is transported to and through the basal-lateral membrane of said epithelial cell.

91. A method for delivering a macromolecule-binding partner complex to a subject, the method comprising contacting an apical surface of a polarized epithelial cell of the subject with a delivery construct comprising a carrier construct non-covalently bound to a binding partner, wherein said carrier construct comprises a receptor binding domain, a transcytosis domain, a cleavable linker and a macromolecule to which the binding partner non-covalently binds to form the macromolecule-binding partner complex, wherein the binding partner binds to the macromolecule with a Kₐ that is at least about 10⁴ M⁻¹, such that said macromolecule-binding partner complex is transported to and through the basal-lateral membrane of said epithelial cell, wherein cleavage at said cleavable linker separates said macromolecule-binding partner complex from the remainder of said delivery construct, and wherein said cleavable linker is cleavable by an enzyme that i) exhibits greater activity at a basal-lateral membrane of a polarized epithelial cell than at an apical membrane of the polarized epithelial cell, or ii) exhibits greater activity in the plasma of said subject than at an apical membrane of the polarized epithelial cell of the subject.

92-122. (canceled)

123. A method for delivering a binding partner to the bloodstream of a subject, the method comprising contacting the delivery construct of claim 1 to an apical surface of a polarized epithelial cell of the subject, such that the binding partner is delivered to the bloodstream of the subject.

124. A method for delivering a macromolecule-binding partner complex to the bloodstream of a subject, the method comprising contacting an apical surface of a polarized epithelial cell of the subject with a delivery construct comprising a carrier construct non-covalently bound to a binding partner, wherein said carrier construct comprises a receptor binding domain, a transcytosis domain, a cleavable linker and a macromolecule to which the binding partner non-covalently binds to form the macromolecule-binding partner complex, wherein the binding partner binds to the macromolecule with a Kₐ that is at least about 10⁴ M⁻¹, such that said macromolecule-binding partner complex is transported to and through the basal-lateral membrane of said epithelial cell, wherein cleavage at said cleavable linker separates said macromolecule-binding partner complex from the remainder of said delivery construct such that the macromolecule-binding partner complex is delivered to the bloodstream, and wherein said cleavable linker is cleavable by an enzyme that i) exhibits greater activity at a basal-lateral membrane of a polarized epithelial cell than at an apical membrane of the polarized epithelial cell, or ii) exhibits greater activity in the plasma of said subject than at an apical membrane of the polarized epithelial cell of the subject.

125-157. (canceled)

158. The delivery construct of claim 33, wherein the macromolecule is human Sck.

159. The delivery construct of claim 34, wherein the macromolecule is human Sck.

160-165. (canceled)
